(19) United States
(12) Patent Application Publication
(10) Pub. No.: US 2017/0355686 A1
(43) Pub. Date: Dec. 14, 2017

(54) NOVEL OXAZOLE COMPOUNDS AS BETA CATENIN MODULATORS AND USES THEREOF

(71) Applicant: NEW YORK UNIVERSITY, New York, NY (US)

(72) Inventors: Ramanuj DASGUPTA, New York, NY (US); Gavara Govinda RAJULU, Bangalore (IN); John K. DICKSON, Apex, NC (US)

(21) Appl. No.: 15/527,228

(22) PCT Filed: Nov. 17, 2015
(86) PCT No.: PCT/US2015/061075

§ 371 Date: May 16, 2017

Related U.S. Application Data
(60) Provisional application No. 62/080,728, filed on Nov. 17, 2014.

(51) Int. Cl. C07D 263/32; C07D 413/12; C07D 413/10

(52) U.S. Cl. CPC C07D 263/32; C07D 413/10; C07D 413/12

(57) ABSTRACT

Oxazole compounds according to formula I:

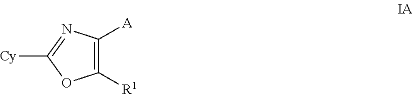

are provided as inhibitors of the Wnt pathway that specifically target the activity of the stabilized pool of β-cat. The compounds may be prepared as pharmaceutical compositions, and may be used for the prevention and treatment of a variety of conditions in mammals including humans, including by way of non-limiting example, cancer, and other conditions related to Wnt pathway dysfunction, including pulmonary fibrosis.

### CROSS REFERENCE TO RELATED APPLICATION

This application claims priority under 35 USC §119(e) from U.S. Provisional Application Serial No. 62/080,728, filed Nov. 17, 2014, which application is herein specifically incorporated by reference in its entirety.

### FIELD OF THE INVENTION

This invention relates to oxazole compounds capable of modulating β-catenin activity and uses of such compounds to modulate the activity of the Wnt/wingless (wg) signaling pathway.

### BACKGROUND OF THE INVENTION

Wnts/wingless (wg) are a family of conserved signaling molecules that have been shown to regulate a plethora of fundamental developmental and cell biological processes, including cell proliferation, differentiation and cell polarity [Miller et al. Oncogene 18, 7860-72 (1999); Polakis. Genes Dev 14, 1837-51 (2000); Wodarz et al. Annu Rev Cell Dev Biol 14, 59-88 (1998)]. Mutations in the Wnt genes or in those genes encoding regulators of the Wnt/wg signaling pathway can cause devastating birth defects, including debilitating abnormalities of the central nervous system, axial skeleton, limbs, and occasionally other organs [Ciruna et al. Nature 439, 220-4 (2006); Grove et al. Development 125, 2315-25 (1998); Jiang et al. Dev Dyn 235, 1152-66 (2006); Kokubu et al. Development 131, 5469-80 (2004); Miyoshi et al. Breast Cancer Res 5, 63-8 (2003); Shu et al. Development 129, 4831-42 (2002); Staal et al. Hematol J 1, 3-6 (2000)]. Aberrant Wnt signaling has also been linked to human disease, such as hepatic, colorectal, breast and skin cancers [Miyoshi et al. supra (2003); Miyoshi et al. Oncogene 21, 5548-56 (2002); Moon et al. Nat Rev Genet 5, 691-701 (2004)].

Wnts/wg encode secreted glycoproteins that activate receptor-mediated pathways leading to numerous transcriptional and cellular responses [Wodarz et al. supra (1998); Moon et al. supra (2004); Nusse. Trends Genet 15, 1-3 (1999)]. The main function of the canonical Wnt pathway is to stabilize the cytoplasmic pool of a key mediator, β-catenin (β-cat)larmadillo (arm), which is otherwise degraded by the proteosome pathway (See FIG. 1). Initially identified as a key player in stabilizing cell-cell adherens junctions, β-cat/arm is also known to act as a transcription factor by forming a complex with the LEF/TCF (Lymphoid Enhancer Factor/T Cell Factor) family of HMG-box (High mobility group) transcription factors. Upon Wnt stimulation, stabilized β-cat/arm translocates to the nucleus, wherein together with LEF/TCF transcription factors, it activates downstream target genes [Miller et al. supra (1999); Staal et al. supra (2000); Nusse. supra (1999); Schweizer et al. Proc Natl Acad Sci USA 100, 5846-51 (2003)]. Catenin responsive transcription (CRT), which is the activation of transcriptional targets of β-cat, has been shown to regulate many aspects of cell growth, proliferation, differentiation and death. The Wnt/wg pathway can also be activated by inhibiting negative regulators such as GSK-3β (Glycogen Synthase Kinase-3β), APC (Adenomatous Polyposis Coli) and Axin that promote β-cat/arm degradation, or by introducing activating mutations in β-cat that render it incapable of interacting with the degradation complex, thus stabilizing its cytosolic pool [Logan et al. Annu Rev Cell Dev Biol 20, 781-810 (2004); Nusse et al. Cell Res 15, 28-32 (2005)]. Wnt/wg signaling can also activate an alternative “non-canonical” pathway that may lead to PKC (Protein Kinase C) and INK (c-Jun N-terminal Kinase) activation resulting in calcium release and cytoskeletal rearrangements [Miller et al. supra (1999)].

At the plasma membrane, Wnt proteins bind to their receptor, belonging to the Frizzled family of proteins and the co-receptor encoded by LDL-related-protein-5, 6 (LRPS, LRP6)/arrow (arr, in Drosophila) [Schweizer et al. BMC Cell Biol 4, 4 (2003); Tamai et al. Mol Cell 13, 149-56 (2004)]. In the absence of the Wnt stimulus, GSK-3β is known to phosphorylate β-cat/arm, which marks it for ubiquitination and subsequent proteosome-mediated degradation. Activation of the receptor/co-receptor complex upon Wnt binding initiates a signal transduction cascade, which results in phosphorylation and subsequent inactivation of GSK-3β24.

Recent evidence has uncovered a new branch in the canonical Wnt/wg pathway whereby β-cat/arm can be stabilized in a GSK-3β independent fashion suggesting that regulated degradation of β-cat/arm (by GSK-3β) is not necessary for Wnt/wg signaling [Tolwinski et al. Dev Cell 4, 407-18 (2003); Tolwinski et al. Trends Genet 20, 177-81 (2004)]. Specifically, upon Wg binding, Arr directly recruits Axin (a scaffold protein which acts as a negative regulator) to the plasma membrane and causes its degradation. As a consequence, Arm no longer binds Axin or the degradation complex, resulting in nuclear accumulation and signaling by β-cat/Arm42.

U.S. Pat. No. 8,252,823, discloses substituted mercaptomethyl-oxazole compounds as β-catenin modulators.

In view of the above, a need exists for therapeutic agents, and corresponding pharmaceutical compositions and related methods of treatment that address conditions causally related to aberrant Wnt pathway activity and CRT activity, and it is toward the fulfillment and satisfaction of that need, that the present invention is directed.

### SUMMARY OF THE INVENTION

Accordingly, the present invention provides a method for preventing, treating or ameliorating in a mammal a disease or condition that is causally related to the aberrant activity of the Wnt pathway in vivo, which comprises administering to the mammal an effective disease-treating or condition-treating amount of a compound according to formula I:

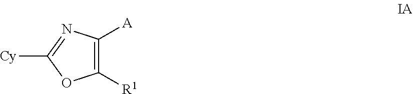

wherein
A is -L¹-X-L²—C(O)—NR^{2a}R^{2b} or -L¹-X-L²—C(O)—OR^{2a};
Cy is substituted or unsubstituted aryl or substituted or unsubstituted heteroaryl;
each of L¹ and L² is independently substituted or unsubstituted C₁-C₇ alkylene or heteroalkylene;
R¹ is hydrogen, halo, or substituted or unsubstituted C₁-C₆ alkyl;
each R^{2a} and R^{2b} is independently selected from H, substituted or unsubstituted alkyl, substituted or unsubstituted cycloalkyl, substituted or unsubstituted aryl, and substituted or unsubstituted heteroaryl; or R^{2a} and R^{2b} are joined together to form a heterocycloalkyl or heteroaryl ring;
X is —O—; or X is S, SO or SO₂;
provided that when the compound is according to formula I, X is S, SO or SO₂, then R^{2a} is H and R^{2b} is ethylene substituted with fluorophenyl, trifluorophenyl, or pyridyl;
or a pharmaceutically acceptable salt, solvate or prodrug thereof;
and stereoisomers, isotopic variants and tautomers thereof.

In certain aspects, the present invention provides a composition of a compound according to formula IA.

In one particular embodiment, with respect to compounds of formula IA, the compound is according to formula I:

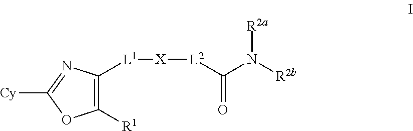

wherein Cy, L¹, X, L², R¹, R^{2a}, and R^{2b} are as in claim 1;
provided that when the compound is according to formula I, X is S, SO or SO₂, then R^{2a} is H and R^{2b} is ethylene substituted with fluorophenyl, trifluorophenyl, or pyridyl;
or a pharmaceutically acceptable salt, solvate or prodrug thereof;
   and stereoisomers, isotopic variants and tautomers thereof.

In another particular embodiment, with respect to compounds of formula IA, the compound is according to formula I′:

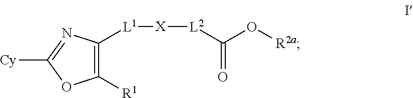

wherein Cy, L¹, X, L², R¹, R^{2a}, and R^{2b} are as in claim 1;
or a pharmaceutically acceptable salt, solvate or prodrug thereof;
   and stereoisomers, isotopic variants and tautomers thereof.

In another particular embodiment, with respect to compounds of formula IA, the compound is according to formula II:

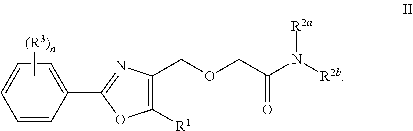

In another particular embodiment, with respect to compounds of formula IA, the compound is according to formula II′:

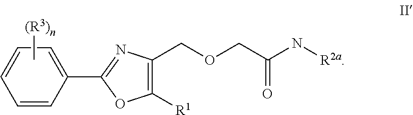

In another particular embodiment, with respect to compounds of formula IA, the compound is according to formula II″:

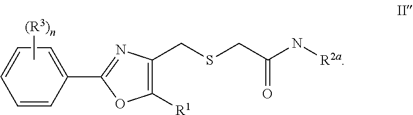

In one particular embodiment, R¹ is substituted or unsubstituted alkyl or halo.

In another particular embodiment, R¹ is Me, Et, i-Pr, n-Pr, n-Bu, F, Cl, Br, or I.

In a more particular embodiment, R¹ is Me.

In a further aspect, the present invention provides pharmaceutical compositions comprising an oxazole compound of the invention, and a pharmaceutically acceptable carrier, excipient or diluent. In this aspect of the invention, the pharmaceutical composition can comprise one or more of the compounds described herein. Moreover, the compounds of the present invention useful in the pharmaceutical compositions and treatment methods disclosed herein are all pharmaceutically acceptable as prepared and used.

In a further aspect, this invention provides the compounds of the invention and other agents for use in the treatment of mammals susceptible to or afflicted with a condition from those listed herein, and particularly, such conditions as may be associated with alterations or aberrations in Wnt/wg pathway signaling.

In addition to the methods of treatment set forth above, the present invention extends to the use of any of the compounds of the invention for the preparation of medicaments that may be administered for such treatments, as well as to such compounds for the treatments disclosed and specified.

A further aspect and object of the invention, is to provide a method of treating a mammal susceptible to or afflicted with a condition from among those listed herein, and particularly, such condition as may be associated with e.g. altered Wnt/wg pathway signaling, by administering to such mammal an effective disease-treating or condition-treating amount of a compound or composition of the invention. Such conditions include, without limitation, pulmonary fibrosis and a variety of hyperproliferative disorders and cancers, including prostate cancer, colon cancer, rectal cancer, breast cancer, skin cancer (e.g., melanoma), liver cancer (e.g., hepatocellular cancer and hepatoblastoma), head and neck cancer, lung cancer (e.g., non-small cell lung cancer), gastric cancer, mesothelioma, Barrett's esophagus, synovial sarcoma, cervical cancer, endometrial ovarian cancer, Wilm's tumor, bladder cancer and leukemia. Additional support for this aspect of the invention is presented in the fact that most cancers of the skin, intestine, and breast epithelial tissue are a result of increased levels of the activated/signaling pool of β-catenin. A number of birth defects are also associated with altered Wnt/wg pathway signaling, including debilitating abnormalities of the central nervous system, axial skeleton, limbs, and occasionally other organs.

Other objects and advantages will become apparent to those skilled in the art from a consideration of the ensuing detailed description, which proceeds with reference to the following illustrative drawings.

### BRIEF DESCRIPTION OF THE DRAWINGS

FIG. 1 shows a plasmid map of Super 8X Topflash.

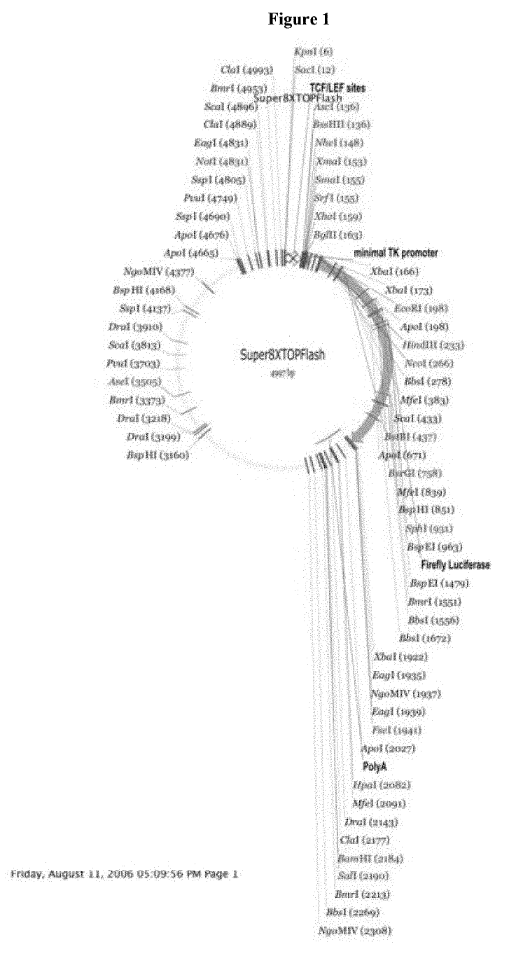

FIG. 2 shows a plasmid map of pPGK-mWnt3A.

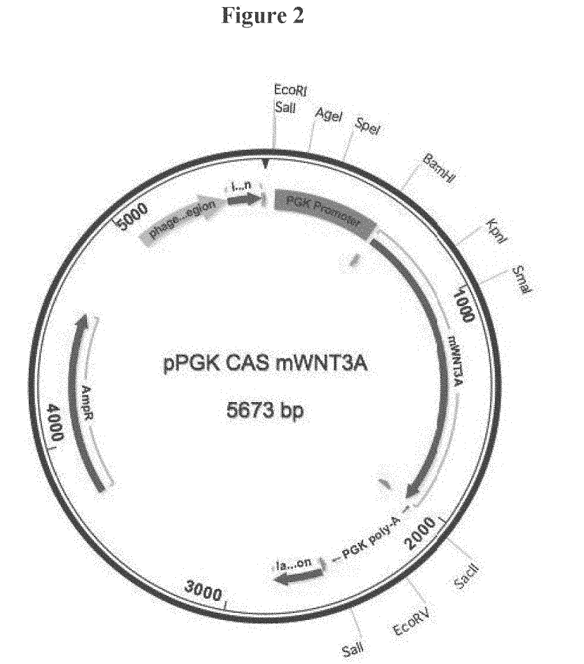

FIG. 3 is a graph showing dose dependent inhibition of the Wnt reporter by Compound iCRT3 in HEK293 cells stimulated with either exogenous (STF) or endogenous Wnt 3a (STF3a). Compound iCRT3 is a reference compound.

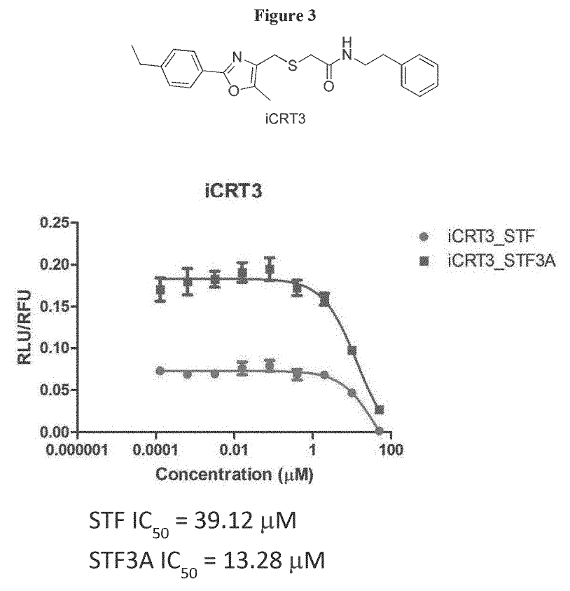

FIG. 4 is a graph showing dose dependent inhibition of the Wnt reporter by iCRT3 analog A3 in HEK293 cells stimulated with either exogenous (STF) or endogenous Wnt 3a (STF3a). Compound A3 is II-1 (Table 2).

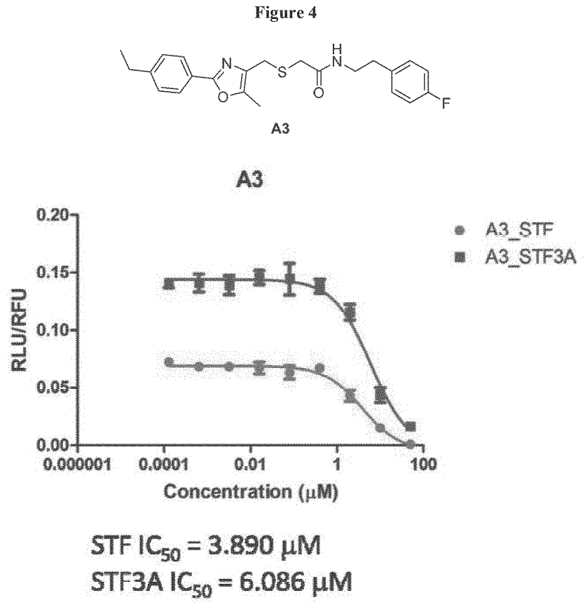

FIG. 5 is a graph showing dose dependent inhibition of the Wnt reporter by iCRT3 analog A12 in HEK293 cells stimulated with either exogenous (STF) or endogenous Wnt 3a (STF3a). Compound A12 is II-7 (Table 2).

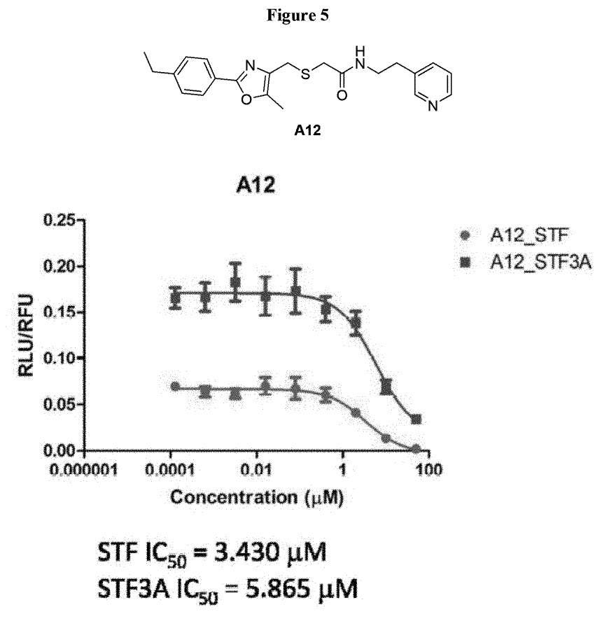

### DETAILED DESCRIPTION OF THE INVENTION

### General Introduction

The Wnt pathway is one of a core set of evolutionarily conserved signaling pathways that regulates many aspects of metazoan development. Misregulation or aberrant regulation of the Wnt pathway can lead to adverse effects as demonstrated by the causal relationship identified between mutations in several components of the pathway and tumorigenesis of the liver, colon, breast and the skin [Wang et al., Cancer Res, 2008. 68(23): 9918-27; Beildeck et al., Exp Cell Res, 2010. 316(11): 1763-72; Yu et al., Prostate, 2009. 69(3): 249-62]. Activating mutations of beta-catenin have also been found in around 5% of prostate cancer [Chesire et al., The Prostate 45, 323 (2000); Voeller et al., Cancer research 58, 2520 (1998)]. Mutation of APC, for example, has been found in 14% of prostate cancer in one study [Gerstein et al., Genes, chromosomes & cancer 34, 9 (2002)] and 3% of prostate cancer in another [Watanabe et al., Japanese journal of clinical oncology 26, 77 (1996)]. One of the most important effectors of the Wnt pathway is encoded by β-catenin (β-cat)/armadillo (arm). Induction by Wnt ligands leads to stabilization of cytosolic β-cat, which subsequently translocates into the nucleus to activate target genes that regulate many aspects of cell proliferation, growth, differentiation and death.

Since catenin responsive transcription (CRT) has been implicated in the genesis of many cancers, this effector step of the pathway provides a good target for developing therapeutics that could modulate Wnt pathway activity, and more particularly, the nuclear activity of β-cat. Notably, the family of compounds disclosed herein comprises inhibitors that specifically target the activity of the signaling pool of β-catenin.

Further to the above, aberrant activation of Wnt signaling has been linked to or causally related with a variety of cancers, including: prostate cancer, colon cancer, rectal cancer, breast cancer, skin cancer (e.g., melanoma), liver cancer (e.g., hepatocellular cancer and hepatoblastoma), head and neck cancer, lung cancer (e.g., non-small cell lung cancer), gastric cancer, mesothelioma, Barrett's esophagus, synovial sarcoma, cervical cancer, endometrial ovarian cancer, Wilm's tumor, bladder cancer and leukemia. See, for example, Luu et al. (2004, Current Cancer Drug Targets 4:653), Lepourcelet et al. (2004, Cancer Cell 5:91), Barker and Clevers (2006, Nature Reviews Drug Discovery 5:997), and Watanabe and Dai (2011, Proc Natl Acad Sci 108:5929), the entire content of each of which is incorporated herein by reference.

In that a strong link has not been established between mutations in the Wnt pathway and prostate cancer, it is informative to review certain aspects of prostate development and prostate cancer development, progression, and treatment to provide insight as to various signaling pathways known to impact this organ. During development, androgens act through the Androgen Receptor (AR) to promote both prostate growth and differentiation. Indeed, maintenance of the prostate organ requires continuous AR and androgen signaling, without which, the prostate regresses. For this reason, aggressive prostate cancer is typically treated with agents that separately block androgen synthesis and inhibit the action of the androgen receptor. However, despite initial regression many cancers recur, making the treatment of what is then called castration-resistant prostate cancer the major challenge in the field. A breakthrough in understanding recurring, resistant disease was the finding that prostate cancer cells become addicted to the AR pathway, and up-regulation of the AR is the major determinate in aggressive castration-resistant prostate cancer [Chen et al., Nat Med, 2004. 10(1): 33-39]. In addition, recent studies show that even under conditions of androgen ablation therapy, prostate cancer cells are able to synthesize androgens locally, through upregulation of androgen synthetic enzymes that direct de novo androgen synthesis or convert adrenal androgens to higher affinity ligands, testosterone and dihydrotestosterone [Titus et al., Clin Cancer Res, 2005. 11(13): 4653-7; Stanbrough et al., Cancer Res, 2006. 66(5): 2815-25; Locke et al., Cancer Res, 2008. 68(15): 6407-15; Montgomery et al., Cancer Res, 2008. 68(11): 4447-54]. The versatility of prostate cancer in evading normal growth controls through altered AR function also encompasses additional mechanisms including generation of novel androgen regulated fusion proteins such as TMPRSS2/ERG [Tomlins et al., Science, 2005. 310(5748): 644-8], production of constitutively active AR splice variants [Dehm et al., Cancer Res, 2008. 68(13): 5469-77] and selection for activating mutations in AR in response to treatment [Steinkamp et al., Cancer Res, 2009. 69(10): 4434-42]. Thus, treatment approaches to develop more effective drugs include agents that block androgen binding to the AR (AR antagonists) such as the MDV3100 compound [Tran et al., Science, 2009. 324(5928): 787-90; Scher et al., Lancet, 2010. 375(9724): 1437-46] or inhibit synthetic enzymes in the androgen synthesis pathway such as abiraterone acetate [Attard et al., Cancer Res, 2009. 69(12): 4937-40]. An additional promising compound blocks the N-terminal transcriptional regulatory domain of the AR [Andersen et al., Cancer Cell, 2010. 17(6): 535-46]. Despite the promise of these and other reagents, they extend life by only by 4-5 months [de Bono et al., N Engl J Med, 2011. 364(21): 1995-2005] and looking forward, the hope is that a variety of agents can be used synergistically or consecutively to further improve life expectancy.

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

‘Acyl’ or ‘Alkanoyl’ refers to a radical —C(O)R²⁰, where R²⁰ is hydrogen, C₁-C₈ alkyl, C₃-C₁₀ cycloalkyl, C₃-C₁₀ cycloalkylmethyl, 4-10 membered heterocycloalkyl, aryl, arylalkyl, 5-10 membered heteroaryl or heteroarylalkyl as defined herein. Representative examples include, but are not limited to, formyl, acetyl, cyclohexylcarbonyl, cyclohexylmethylcarbonyl, benzoyl and benzylcarbonyl. Exemplary ‘acyl’ groups are —C(O)H, —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocycloalkyl), wherein t is an integer from 0 to 4.

‘Substituted Acyl’ or ‘Substituted Alkanoyl’ refers to a radical —C(O)R²¹, wherein R²¹ is independently
C₁-C₈ alkyl, substituted with halo or hydroxy; or
C₃-C₁₀ cycloalkyl, 4-10 membered heterocycloalkyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Acylamino’ refers to a radical —NR²²C(O)R²³, where R²² is hydrogen, C₁-C₈ alkyl, C₃-C₁₀ cycloalkyl, 4-10 membered heterocycloalkyl, C₆-C₁₀ aryl, arylalkyl, 5-10 membered heteroaryl or heteroarylalkyl and R²³ is hydrogen, C₁-C₈ alkyl, C₃-C₁₀ cycloalkyl, 4-10 membered heterocycloalkyl, C₆-C₁₀ aryl, arylalkyl, 5-10 membered heteroaryl or heteroarylalkyl, as defined herein. Exemplary ‘acylamino’ include, but are not limited to, formylamino, acetylamino, cyclohexylcarbonylamino, cyclohexylmethyl-carbonylamino, benzoylamino and benzylcarbonylamino. Particular exemplary ‘acylamino’ groups are —NR²⁴C(O)—C₁-C₈ alkyl, —NR²⁴C(O)—(CH₂)ₜ(C₆-C₁₀ aryl), (O)—(CH₂)ₜ(5-10 membered heteroaryl), —NR²⁴C(O)—(CH₂)ₜ(C₃-C₁₀ cycloalkyl), and —NR²⁴C(O)—(CH₂)ₜ(4-10 membered heterocycloalkyl), wherein t is an integer from 0 to 4, and each R²⁴ independently represents H or C₁-C₈ alkyl.

‘Substituted Acylamino’ refers to a radical —NR²⁵C(O)R²⁶, wherein:

R²⁵ is independently
H, C₁-C₈ alkyl, substituted with halo or hydroxy; or
C₃-C₁₀ cycloalkyl, 4-10 membered heterocycloalkyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; and

R²⁶ is independently
H, C₁-C₈ alkyl, substituted with halo or hydroxy; or
C₃-C₁₀ cycloalkyl, 4-10 membered heterocycloalkyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl;

provided at least one of R²⁵ and R²⁶ is other than H.

‘Acyloxy’ refers to a radical —OC(O)R²⁷, where R²⁷ is hydrogen, C₁-C₈ alkyl, C₃-C₁₀ cycloalkyl, C₃-C₁₀ cycloalkylmethyl, 4-10 membered heterocycloalkyl, aryl, arylalkyl, 5-10 membered heteroaryl or heteroarylalkyl as defined herein. Representative examples include, but are not limited to, formyl, acetyl, cyclohexylcarbonyl, cyclohexylmethylcarbonyl, benzoyl and benzylcarbonyl. Exemplary ‘acyl’ groups are —C(O)H, —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocycloalkyl), wherein t is an integer from 0 to 4.

‘Substituted Acyloxy’ refers to a radical —OC(O)R²⁸, wherein R²⁸ is independently
C₁-C₈ alkyl, substituted with halo or hydroxy; or
C₃-C₁₀ cycloalkyl, 4-10 membered heterocycloalkyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Alkoxy’ refers to the group —OR²⁹ where R²⁹ is C₁-C₈ alkyl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

‘Substituted alkoxy’ refers to an alkoxy group substituted with one or more of those groups recited in the definition of “substituted” herein, and particularly refers to an alkoxy group having 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocycloalkyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups are —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are OCF₃, OCH₂CF₃, OCH₂Ph, OCH₂-cyclopropyl, OCH₂CH₂OH, and OCH₂CH₂NMe₂.

‘Alkoxycarbonyl’ refers to a radical —C(O)—OR³⁰ where R³⁰ represents an C₁-C₈ alkyl, C₃-C₁₀ cycloalkyl, C₃-C₁₀ cycloalkylalkyl, 4-10 membered heterocycloalkylalkyl, aralkyl, or 5-10 membered heteroarylalkyl as defined herein. Exemplary “alkoxycarbonyl” groups are C(O)O—C₁-C₈ alkyl, —C(O)O—(CH₂)ₜ(C₆-C₁₀ aryl), —C(O)O—(CH₂)ₜ(5-10 membered heteroaryl), —C(O)O—(CH₂)ₜ(C₃-C₁₀ cycloalkyl), and —C(O)O—(CH₂)ₜ(4-10 membered heterocycloalkyl), wherein t is an integer from 1 to 4.

‘Substituted Alkoxycarbonyl’ refers to a radical —C(O)—OR³¹ where R³¹ represents:
C₁-C₈ alkyl, C₃-C₁₀ cycloalkyl, C₃-C₁₀ cycloalkylalkyl, or 4-10 membered heterocycloalkylalkyl, each of which is substituted with halo, substituted or unsubstituted amino, or hydroxy; or
C₆-C₁₀ aralkyl, or 5-10 membered heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl.

‘Aryloxycarbonyl’ refers to a radical —C(O)—OR³² where R³² represents an C₆-C₁₀ aryl, as defined herein. Exemplary “aryloxycarbonyl” groups is —C(O)O—(C₆-C₁₀ aryl).

‘Substituted Aryloxycarbonyl’ refers to a radical —C(O)—OR³³ where R³³ represents
C₆-C₁₀ aryl, substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl.

‘Heteroaryloxycarbonyl’ refers to a radical —C(O)—OR³⁴ where R³⁴ represents a 5-10 membered heteroaryl, as defined herein. An exemplary “aryloxycarbonyl” group is —C(O)O-(5-10 membered heteroaryl).

‘Substituted Heteroaryloxycarbonyl’ refers to a radical —C(O)—OR³⁵ where R³⁵ represents:
5-10 membered heteroaryl, substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl.

“Alkoxycarbonylamino” refers to the group —NR³⁶C(O)OR³⁷, where R³⁶ is hydrogen, C₁-C₈ alkyl, C₃-C₁₀ cycloalkyl, C₃-C₁₀ cycloalkylmethyl, 4-10 membered heterocycloalkyl, aryl, arylalkyl, 5-10 membered heteroaryl or heteroarylalkyl as defined herein, and R³⁷ is C₁-C₈ alkyl, C₃-C₁₀ cycloalkyl, C₃-C₁₀ cycloalkylmethyl, 4-10 membered heterocycloalkyl, aryl, arylalkyl, 5-10 membered heteroaryl or heteroarylalkyl as defined herein.

‘Alkyl’ means straight or branched aliphatic hydrocarbon having 1 to 20 carbon atoms. Particular alkyl has 1 to 12 carbon atoms. More particular is lower alkyl which has 1 to 6 carbon atoms. A further particular group has 1 to 4 carbon atoms. Exemplary straight chained groups include methyl, ethyl n-propyl, and n-butyl. Branched means that one or more lower alkyl groups such as methyl, ethyl, propyl or butyl is attached to a linear alkyl chain, exemplary branched chain groups include isopropyl, iso-butyl, t-butyl and isoamyl.

‘Substituted alkyl’ refers to an alkyl group as defined above substituted with one or more of those groups recited in the definition of “substituted” herein, and particularly refers to an alkyl group having 1 or more substituents, for instance from 1 to 5 substituents, and particularly from 1 to 3 substituents, in particular 1 substituent, selected from the group consisting of acyl, acylamino, acyloxy (—O-acyl or —OC(O)R²⁰), alkoxy, alkoxycarbonyl, alkoxycarbonylamino (—NR″-alkoxycarbonyl or —NH—C(O)—OR²⁷), amino, substituted amino, aminocarbonyl (carbamoyl or amido or —C(O)—NR″₂), aminocarbonylamino (—NR″—C(O)—NR″₂), aminocarbonyloxy (—O—C(O)—NR″₂), aminosulfonyl, sulfonylamino, aryl, aryloxy, azido, carboxyl, cyano, cycloalkyl, halogen, hydroxy, heteroaryl, nitro, thiol, —S-alkyl, —S-aryl, —S(O)-alkyl, —S(O)-aryl, —S(O)₂-alkyl, and —S(O)₂-aryl. In a particular embodiment ‘substituted alkyl’ refers to a C₁-C₈ alkyl group substituted with halo, cyano, nitro, trifluoromethyl, trifluoromethoxy, azido, —NR′″SO₂R″, —SO₂NR″R′″, —C(O)R″, —C(O)OR″, —OC(O)R″, —NR′″C(O)R″, —C(O)NR″R′″, —NR″R′″, or —(CR′″R″″)ₘOR′″; wherein each R″ is independently selected from H, C₁-C₈ alkyl, —(CH₂)ₜ(C₆-C₁₀ aryl), —(CH₂)ₜ(5-10 membered heteroaryl), —(CH₂)ₜ(C₃-C₁₀ cycloalkyl), and —(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Each of R′″ and R″″ independently represents H or C₁-C₈ alkyl.

“Alkylene” refers to divalent saturated alkene radical groups having 1 to 11 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

‘Substituted alkylene’ refers to those groups recited in the definition of “substituted” herein, and particularly refers to an alkylene group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, amino-carbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkenyl” refers to monovalent olefinically unsaturated hydrocarbyl groups preferably having 2 to 11 carbon atoms, particularly, from 2 to 8 carbon atoms, and more particularly, from 2 to 6 carbon atoms, which can be straight-chained or branched and having at least 1 and particularly from 1 to 2 sites of olefinic unsaturation. Particular alkenyl groups include ethenyl (—CH═CH₂), n-propenyl (—CH₂CH═CH₂), isopropenyl (—C(CH₃)═CH₂), vinyl and substituted vinyl, and the like.

“Substituted alkenyl” refers to those groups recited in the definition of “substituted” herein, and particularly refers to an alkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkenylene” refers to divalent olefinically unsaturated hydrocarbyl groups particularly having up to about 11 carbon atoms and more particularly 2 to 6 carbon atoms which can be straight-chained or branched and having at least 1 and particularly from 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

“Alkynyl” refers to acetylenically or alkynically unsaturated hydrocarbyl groups particularly having 2 to 11 carbon atoms, and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Substituted alkynyl” refers to those groups recited in the definition of “substituted” herein, and particularly refers to an alkynyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

‘Amino’ refers to the radical —NH₂.

‘Substituted amino’ refers to an amino group substituted with one or more of those groups recited in the definition of ‘substituted’ herein, and particularly refers to the group —N(R³⁸)₂ where each R³⁸ is independently selected from:
hydrogen, C₁-C₈ alkyl, C₆-C₁₀ aryl, 5-10 membered heteroaryl, 4-10 membered heterocycloalkyl, or C₃-C₁₀ cycloalkyl; or
C₁-C₈ alkyl, substituted with halo or hydroxy; or
—(CH₂)ₜ(C₆-C₁₀ aryl), —(CH₂)ₜ(5-10 membered heteroaryl), —(CH₂)ₜ(C₃-C₁₀ cycloalkyl) or —(CH₂)ₜ(4-10 membered heterocycloalkyl) wherein t is an integer between 0 and 8, each of which is substituted by unsubstituted C₁-C₄ alkyl, halo, unsubstituted alkoxy, unsubstituted C₁-C₄ haloalkyl, unsubstituted C₁-C₄ hydroxyalkyl, or unsubstituted C₁-C₄ haloalkoxy or hydroxy; or
both R³⁸ groups are joined to form an alkylene group.
   When both R³⁸ groups are hydrogen, —N(R³⁸)₂ is an amino group. Exemplary ‘substituted amino’ groups are —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocycloalkyl), wherein t is an integer from 0 to 4, each R³⁹ independently represents H or C₁-C₈ alkyl; and any alkyl groups present, may themselves be substituted by halo, substituted or unsubstituted amino, or hydroxy; and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term “substituted amino” includes the groups alkylamino, substituted alkylamino, alkylarylamino, substituted alkylarylamino, arylamino, substituted arylamino, dialkylamino and substituted dialkylamino as defined below.

‘Alkylamino’ refers to the group —NHR⁴⁰, wherein R⁴⁰ is C₁-C₈ alkyl;

‘Substituted Alkylamino’ refers to the group —NHR⁴¹, wherein R⁴¹ is C₁-C₈ alkyl; and the alkyl group is substituted with halo, substituted or unsubstituted amino, hydroxy, C₃-C₁₀ cycloalkyl, 4-10 membered heterocycloalkyl, C₆-C₁₀ aryl, 5-10 membered heteroaryl, aralkyl or heteroaralkyl; and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Alkylarylamino’ refers to the group —NR⁴²R⁴³, wherein R⁴² is aryl and R⁴³ is C₁-C₈ alkyl.

‘Substituted Alkylarylamino’ refers to the group —NR⁴⁴R⁴⁵, wherein R⁴⁴ is aryl and R⁴⁵ is C₁-C₈ alkyl; and the alkyl group is substituted with halo, substituted or unsubstituted amino, hydroxy, C₃-C₁₀ cycloalkyl, 4-10 membered heterocycloalkyl, C₆-C₁₀ aryl, 5-10 membered heteroaryl, aralkyl or heteroaralkyl; and any aryl, heteroaryl, cycloalkyl or heterocycloalkyl groups present, may themselves be substituted by unsubstituted C₁-C₄ alkyl, halo, cyano, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Arylamino’ means a radical —NHR⁴⁶ where R⁴⁶ is selected from C₆-C₁₀ aryl and 5-10 membered heteroaryl as defined herein.

‘Substituted Arylamino’ refers to the group —NHR⁴⁷, wherein R⁴⁷ is independently selected from C₆-C₁₀ aryl and 5-10 membered heteroaryl; and any aryl or heteroaryl groups present, may themselves be substituted by unsubstituted C₁-C₄ alkyl, halo, cyano, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Dialkylamino’ refers to the group —NR⁴⁸R⁴⁹, wherein each of R⁴⁸ and R⁴⁹ are independently selected from C₁-C₈ alkyl.

‘Substituted Dialkylamino’ refers to the group —NR⁵⁰R⁵¹, wherein each of R⁵⁹ and R⁵¹ are independently selected from C₁-C₈ alkyl; and at least one of the alkyl groups is independently substituted with halo, hydroxy, C₃-C₁₀ cycloalkyl, 4-10 membered heterocycloalkyl, C₆-C₁₀ aryl, 5-10 membered heteroaryl, aralkyl or heteroaralkyl; and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-₄ haloalkyl, unsubstituted C₁-C₄ hydroxyalkyl, or unsubstituted C₁-C₄ haloalkoxy or hydroxy.

‘Diarylamino’ refers to the group —NR⁵²R⁵³, wherein each of R⁵² and R⁵³ are independently selected from C₆-C₁₀ aryl.

“Aminosulfonyl” or “Sulfonamide” refers to the radical —S(O₂)NH₂.

“Substituted aminosulfonyl” or “substituted sulfonamide” refers to a radical such as —S(O₂)N(R⁵⁴)₂ wherein each R⁵⁴⁸ is independently selected from:
H, C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; provided that at least one R⁵⁴ is other than H.

Exemplary ‘substituted aminosulfonyl’ or ‘substituted sulfonamide’ groups are —S(O₂)N(R⁵⁵)—C₁-C₈ alkyl, —S(O₂)N(R⁵⁵)—(CH₂)ₜ(C₆-C₁₀ aryl), —S(O₂)N(R⁵⁵)—(CH₂)ₜ(5-10 membered heteroaryl), —S(O₂)N(R⁵⁵)—(CH₂)ₜ(C₃-C₁₀ cycloalkyl), and —S(O₂)N(R⁵⁵)—(CH₂)ₜ(4-10 membered heterocycloalkyl), wherein t is an integer from 0 to 4; each R⁵⁵ independently represents H or C₁-C₈ alkyl; and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Aralkyl’ or ‘arylalkyl’ refers to an alkyl group, as defined above, substituted with one or more aryl groups, as defined above. Particular aralkyl or arylalkyl groups are alkyl groups substituted with one aryl group.

‘Substituted Aralkyl’ or ‘substituted arylalkyl’ refers to an alkyl group, as defined above, substituted with one or more aryl groups; and at least one of the aryl groups present, may themselves be substituted by unsubstituted C₁-C₄ alkyl, halo, cyano, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Aryl’ refers to a monovalent aromatic hydrocarbon group derived by the removal of one hydrogen atom from a single carbon atom of a parent aromatic ring system. In particular aryl refers to an aromatic ring structure, mono-cyclic or poly-cyclic that includes from 5 to 12 ring members, more usually 6 to 10. Where the aryl group is a monocyclic ring system it preferentially contains 6 carbon atoms. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene and trinaphthalene. Particularly aryl groups include phenyl, naphthyl, indenyl, and tetrahydronaphthyl.

‘Substituted Aryl’ refers to an aryl group substituted with one or more of those groups recited in the definition of ‘substituted’ herein, and particularly refers to an aryl group that may optionally be substituted with 1 or more substituents, for instance from 1 to 5 substituents, particularly 1 to 3 substituents, in particular 1 substituent. Particularly, ‘Substituted Aryl’ refers to an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

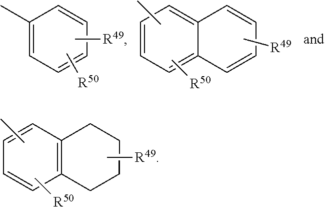

In these formulae one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocycloalkyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O or S. R⁶⁰, and R⁶¹ are independently hydrogen, C₁-C₈ alkyl, C₁-C₄ haloalkyl, C₃-C₁₀ cycloalkyl, 4-10 membered heterocycloalkyl, C₆-C₁₀ aryl, substituted aryl, 5-10 membered heteroaryl.

“Fused Aryl” refers to an aryl having two of its ring carbon in common with a second aryl ring or with an aliphatic ring.

‘Arylalkyloxy’ refers to an —O-alkylaryl radical where alkylaryl is as defined herein.

‘Substituted Arylalkyloxy’ refers to an —O-alkylaryl radical where alkylaryl is as defined herein; and any aryl groups present, may themselves be substituted by unsubstituted C₁-C₄ alkyl, halo, cyano, unsubstituted C₁-C₄ alkoxy, unsubstituted C₁₋₄ haloalkyl, unsubstituted C₁-C₄ hydroxyalkyl, or unsubstituted C₁-C₄ haloalkoxy or hydroxy.

‘Azido’ refers to the radical —N₃.

‘Carbamoyl or amido’ refers to the radical —C(O)NH₂.

‘Substituted Carbamoyl or substituted amido’ refers to the radical —C(O)N(R⁶²)₂ wherein each R⁶² is independently
H, C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy;
   provided that at least one R⁶² is other than H.
   Exemplary ‘Substituted Carbamoyl’ groups are —C(O)NR⁶⁴—C₁-C₈ alkyl, —C(O)NR⁶⁴—(CH₂)ₜ(C₆-C₁₀ aryl), —C(O)N⁶⁴—(CH₂)ₜ(5-10 membered heteroaryl), —C(O)NR⁶⁴—(CH₂)ₜ(C₃-C₁₀ cycloalkyl), and —C(O)NR⁶—(CH₂)ₜ(4-10 membered heterocycloalkyl), wherein t is an integer from 0 to 4, each R⁶⁴ independently represents H or C₁-C₈ alkyl and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Carboxy’ refers to the radical —C(O)OH.

‘Cycloalkyl’ refers to cyclic non-aromatic hydrocarbyl groups having from 3 to 10 carbon atoms. Such cycloalkyl groups include, by way of example, single ring structures such as cyclopropyl, cyclobutyl, cyclopentyl, and cyclooctyl.

‘Substituted cycloalkyl’ refers to a cycloalkyl group as defined above substituted with one or more of those groups recited in the definition of ‘substituted’ herein, and particularly refers to a cycloalkyl group having 1 or more substituents, for instance from 1 to 5 substituents, and particularly from 1 to 3 substituents, in particular 1 substituent

‘Cyano’ refers to the radical —CN.

‘Halo’ or ‘halogen’ refers to fluoro (F), chloro (Cl), bromo (Br) and iodo (I). Particular halo groups are either fluoro or chloro.

‘Hetero’ when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, cycloalkenyl, e.g. cycloheteroalkenyl, and the like having from 1 to 5, and particularly from 1 to 3 heteroatoms.

‘Heteroaryl’ means an aromatic ring structure, mono-cyclic or polycyclic, that includes one or more heteroatoms and 5 to 12 ring members, more usually 5 to 10 ring members. The heteroaryl group can be, for example, a five membered or six membered monocyclic ring or a bicyclic structure formed from fused five and six membered rings or two fused six membered rings or, by way of a further example, two fused five membered rings. Each ring may contain up to four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five. Examples of five membered monocyclic heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups. Examples of six membered monocyclic heteroaryl groups include but are not limited to pyridine, pyrazine, pyridazine, pyrimidine and triazine. Particular examples of bicyclic heteroaryl groups containing a five membered ring fused to another five membered ring include but are not limited to imidazothiazole and imidazoimidazole. Particular examples of bicyclic heteroaryl groups containing a six membered ring fused to a five membered ring include but are not limited to benzfuran, benzthiophene, benzimidazole, benzoxazole, isobenzoxazole, benzisoxazole, benzthiazole, benzisothiazole, isobenzofuran, indole, isoindole, isoindolone, indolizine, indoline, isoindoline, purine (e.g., adenine, guanine), indazole, pyrazolopyrimidine, triazolopyrimidine, benzodioxole and pyrazolopyridine groups. Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups. Particular heteroaryl groups are those derived from thiophene, pyrrole, benzothiophene, benzofuran, indole, pyridine, quinoline, imidazole, oxazole and pyrazine.

Examples of representative heteroaryls include the following:

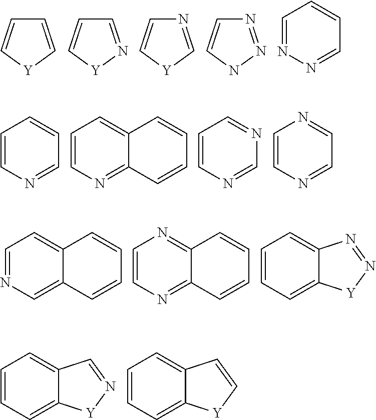

wherein each Y is selected from carbonyl, N, NR⁶⁵, O and S; and R⁶⁵ is independently hydrogen, C₁-C₈ alkyl, C₃-C₁₀ cycloalkyl, 4-10 membered heterocycloalkyl, C₆-C₁₀ aryl, and 5-10 membered heteroaryl.

Examples of representative aryl having hetero atoms containing substitution include the following:

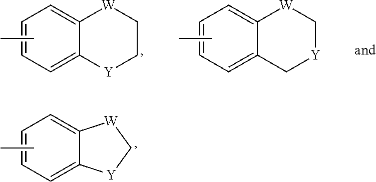

wherein each W is selected from C(R⁶⁶)₂, NR⁶⁶, O and S; and each Y is selected from carbonyl, NR⁶⁶, O and S; and R⁶⁶ is independently hydrogen, C₁-C₈ alkyl, C₃-C₁₀ cycloalkyl, 4-10 membered heterocycloalkyl, C₆-C₁₀ aryl, and 5-10 membered heteroaryl.

As used herein, the term ‘heterocycloalkyl’ refers to a 4-10 membered, stable heterocyclic non-aromatic ring and/or including rings containing one or more heteroatoms independently selected from N, O and S, fused thereto. A fused heterocyclic ring system may include carbocyclic rings and need only include one heterocyclic ring. Examples of heterocyclic rings include, but are not limited to, morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Further examples include thiomorpholine and its S-oxide and S,S-dioxide (particularly thiomorpholine). Still further examples include azetidine, piperidone, piperazone, and N-alkyl piperidines such as N-methyl piperidine. Particular examples of heterocycloalkyl groups are shown in the following illustrative examples:

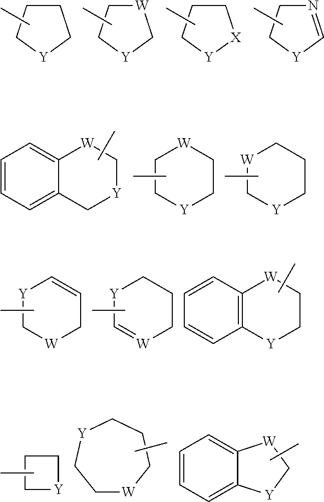

wherein each W is selected from CR⁶⁷, C(R⁶⁷)₂, NR⁶⁷, O and S; and each Y is selected from NR⁶⁷, O and S; and R⁶⁷ is independently hydrogen, C₁-C₈ alkyl, C₃-C₁₀ cycloalkyl, 4-10 membered heterocycloalkyl, C₆-C₁₀ aryl, 5-10 membered heteroaryl, These heterocycloalkyl rings may be optionally substituted with one or more groups selected from the group consisting of acyl, acylamino, acyloxy, alkoxy, alkoxycarbonyl, alkoxycarbonylamino, amino, substituted amino, aminocarbonyl (carbamoyl or amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

‘Hydroxy’ refers to the radical —OH.

‘Nitro’ refers to the radical —NO₂.

‘Substituted’ refers to a group in which one or more hydrogen atoms are each independently replaced with the same or different substituent(s). Typical substituents may be selected from the group consisting of:
halogen, —R⁶⁸, —O⁻, ═O, —OR⁶⁸, —SR⁶⁸, —S⁻, ═S, —NR⁶⁸R⁶⁹, ═NR⁶⁸, —CCl₃, —CF₃, —CN, —OCN, —SCN, —NO, —NO₂, ═N₂, —N₃, —S(O)₂O⁻, —S(O)₂OH, —S(O)₂R⁶⁸, —OS(O₂)O⁻, —OS(O)₂R⁶⁸, —P(O)(O⁻)₂, —P(O)(OR⁶⁸)(O⁻), —OP(O)(OR⁶⁸)(OR⁶⁹), —C(O)R⁶⁸, —C(S)R⁶⁸, —C(O)OR⁶⁸, —C(O)NR⁶⁸R⁶⁹, —C(O)O⁻, —C(S)OR⁶⁸, —NR⁷⁰C(O)NR⁶⁸R⁶⁹, —NR⁷⁰C(S)NR⁶⁸R⁶⁹, —NR⁷¹C(NR⁷⁰)NR⁶⁸R⁶⁹ and —C(NR⁷⁰)NR⁶⁸R⁶⁹;
wherein each R⁶⁸, R⁶⁹, R⁷⁰ and R⁷¹ are independently:
hydrogen, C₁-C₈ alkyl, C₆-C₁₀ aryl, arylalkyl, C₃-C₁₀ cycloalkyl, 4-10 membered heterocycloalkyl, 5-10 membered heteroaryl, heteroarylalkyl; or
C₁-C₈ alkyl substituted with halo or hydroxy; or
C₆-C₁₀ aryl, 5-10 membered heteroaryl, C₆-C₁₀ cycloalkyl or 4-10 membered heterocycloalkyl each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.
   In a particular embodiment, substituted groups are substituted with one or more substituents, particularly with 1 to 3 substituents, in particular with one substituent group. In a further particular embodiment the substituent group or groups are selected from halo, cyano, nitro, trifluoromethyl, trifluoromethoxy, azido, —NR⁷²SO₂R⁷³, —SO₂NR⁷³R⁷², —C(O)R⁷³, —C(O)OR⁷³, —OC(O)R⁷³, —NR⁷²C(O)R⁷³, —C(O)NR⁷³R⁷², —NR⁷³R⁷², —(CR⁷²R⁷²)ₘOR⁷², wherein, each R⁷³ is independently selected from H, C₁-C₈ alkyl, —(CH₂)ₜ(C₆-C₁₀ aryl), —(CH₂)ₜ(5-10 membered heteroaryl), —(CH₂)ₜ(C₃-C₁₀ cycloalkyl), and —(CH₂)ₜ(4-10 membered heterocycloalkyl), wherein t is an integer from 0 to 4; and
any alkyl groups present, may themselves be substituted by halo or hydroxy; and
any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Each R″ independently represents H or C₁-C₆alkyl.

‘Substituted sulfanyl’ refers to the group —SR⁷⁴, wherein R⁷⁴ is selected from:
C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo, substituted or unsubstituted amino, or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

Exemplary ‘substituted sulfanyl’ groups are —S—(C₁-C₈ alkyl) and —S—(C₃-C₁₀ cycloalkyl), —S—(CH₂)ₜ(C₆-C₁₀ aryl), —S—(CH₂)ₜ(5-10 membered heteroaryl), —S—(CH₂)ₜ(C₃-C₁₀ cycloalkyl), and —S—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. The term ‘substituted sulfanyl’ includes the groups ‘alkylsulfanyl’ or ‘alkylthio’, ‘substituted alkylthio’ or ‘substituted alkylsulfanyl’, ‘cycloalkylsulfanyl’ or ‘cycloalkylthio’, ‘substituted cycloalkylsulfanyl’ or ‘substituted cycloalkylthio’, ‘arylsulfanyl’ or ‘arylthio’ and ‘heteroarylsulfanyl’ or ‘heteroarylthio’ as defined below.

‘Alkylthio’ or ‘Alkylsulfanyl’ refers to a radical —SR⁷⁵ where R⁷⁵ is a C₁-C₈ alkyl or group as defined herein. Representative examples include, but are not limited to, methylthio, ethylthio, propylthio and butylthio.

‘Substituted Alkylthio’or ‘substituted alkylsulfanyl’ refers to the group —SR⁷⁶ where R⁷⁶ is a C₁-C₈ alkyl, substituted with halo, substituted or unsubstituted amino, or hydroxy.

‘Cycloalkylthio’ or ‘Cycloalkylsulfanyl’ refers to a radical —SR⁷⁷ where R⁷⁷ is a C₃-C₁₀ cycloalkyl or group as defined herein. Representative examples include, but are not limited to, cyclopropylthio, cyclohexylthio, and cyclopentylthio.

‘Substituted cycloalkylthio’ or ‘substituted cycloalkylsulfanyl’ refers to the group —R⁷⁸ where R⁷⁸ is a C₃-C₁₀ cycloalkyl, substituted with halo, substituted or unsubstituted amino, or hydroxy.

‘Arylthio’ or ‘Arylsulfanyl’ refers to a radical —SR⁷⁹ where R⁷⁹ is a C₆-C₁₀ aryl group as defined herein.

‘Heteroarylthio’ or ‘Heteroarylsulfanyl’ refers to a radical —SR⁸⁰ where R⁸⁰ is a 5-10 membered heteroaryl group as defined herein.

‘Substituted sulfinyl’ refers to the group —S(O)R⁸¹, wherein R⁸¹ is selected from:
C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo, substituted or unsubstituted amino, or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁₋₄ hydroxyalkyl, or unsubstituted C₁-C₄ haloalkoxy or hydroxy.

Exemplary ‘substituted sulfinyl’ groups are —S(O)—(C₁-C₈ alkyl) and —S(O)—(C₃-C₁₀ cycloalkyl), —S(O)—(CH₂)ₜ(C₆-C₁₀ aryl), —S(O)—(CH₂)ₜ(5-10 membered heteroaryl), —S(O)—(CH₂)ₜ(C₃-C₁₀ cycloalkyl), and —S(O)—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. The term substituted sulfinyl includes the groups ‘alkylsulfinyl’, ‘substituted alkylsulfinyl’, ‘cycloalkylsulfinyl’, ‘substituted cycloalkylsulfinyl’, ‘arylsulfinyl’ and ‘heteroarylsulfinyl’ as defined herein.

‘Alkylsulfinyl’ refers to a radical —S(O)R⁸² where R⁸² is a C₁-C₈ alkyl group as defined herein. Representative examples include, but are not limited to, methylsulfinyl, ethylsulfinyl, propylsulfinyl and butylsulfinyl.

‘Substituted Alkylsulfinyl’ refers to a radical —S(O)R⁸³ where R⁸³ is a C₁-C₈ alkyl group as defined herein. substituted with halo, substituted or unsubstituted amino, or hydroxy.

‘Cycloalkylsulfinyl’ refers to a radical —S(O)R⁸⁴ where R⁸⁴ is a C₃-C₁₀ cycloalkyl or group as defined herein. Representative examples include, but are not limited to, cyclopropylsulfinyl, cyclohexylsulfinyl, and cyclopentylsulfinyl. Exemplary ‘cycloalkylsulfinyl’ groups are S(O)—C₃-C₁₀ cycloalkyl.

‘Substituted cycloalkylsulfinyl’ refers to the group —S(O)R⁸⁵ where R⁸⁵ is a C₃-C₁₀ cycloalkyl, substituted with halo, substituted or unsubstituted amino, or hydroxy.

‘Arylsulfinyl’ refers to a radical —S(O)R⁸⁶ where R⁸⁶ is a C₆-C₁₀ aryl group as defined herein.

‘Heteroarylsulfinyl’ refers to a radical —S(O)R⁸⁷ where R⁸⁷ is a 5-10 membered heteroaryl group as defined herein.

‘Substituted sulfonyl’ refers to the group —S(O)₂R⁸⁸, wherein R⁸⁸ is selected from:
C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo, substituted or unsubstituted amino, or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

Exemplary ‘substituted sulfonyl’ groups are —S(O)₂—(C₁-C₈ alkyl) and —S(O)₂—(C₃-C₁₀ cycloalkyl), —S(O)₂—(CH₂)ₜ(C₆-C₁₀ aryl), —S(O)₂—(CH₂)ₜ(5-10 membered heteroaryl), —S(O)₂—(CH₂)ₜ(C₃-C₁₀ cycloalkyl), and —S(O)₂—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. The term substituted sulfonyl includes the groups alkylsulfonyl, substituted alkylsulfonyl, cycloalkylsulfonyl, substituted cycloalkylsulfonyl, arylsulfonyl and heteroarylsulfonyl.

‘Alkylsulfonyl’ refers to a radical —S(O)₂R⁸⁹ where R⁸⁹ is an C₁-C₈ alkyl group as defined herein. Representative examples include, but are not limited to, methylsulfonyl, ethylsulfonyl, propylsulfonyl and butylsulfonyl.

‘Substituted Alkylsulfonyl’ refers to a radical —S(O)₂R⁹⁰ where R⁹⁰ is an C₁-C₈ alkyl group as defined herein, substituted with halo, substituted or unsubstituted amino, or hydroxy.

‘Cycloalkylsulfonyl’ refers to a radical —S(O)₂R⁹¹ where R⁹¹ is a C₃-C₁₀ cycloalkyl or group as defined herein. Representative examples include, but are not limited to, cyclopropylsulfonyl, cyclohexylsulfonyl, and cyclopentylsulfonyl.

‘Substituted cycloalkylsulfonyl’ refers to the group —S(O)₂R⁹² where R⁹² is a C₃-C₁₀ cycloalkyl, substituted with halo, substituted or unsubstituted amino, or hydroxy.

‘Arylsulfonyl’ refers to a radical —S(O)₂R⁹³ where R⁹³ is an C₆-C₁₀ aryl group as defined herein.

‘Heteroarylsulfonyl’ refers to a radical —S(O)₂R⁹⁴ where R⁹⁴ is an 5-10 membered heteroaryl group as defined herein.

‘Sulfo’ or ‘sulfonic acid’ refers to a radical such as —SO₃H.

‘Substituted sulfo’ or ‘sulfonic acid ester’ refers to the group —S(O)₂OR⁹⁵, wherein R⁹⁵ is selected from:
C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo, substituted or unsubstituted amino, or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

Exemplary ‘Substituted sulfo’ or ‘sulfonic acid ester’ groups are —S(O)₂—O—(C₁-C₈ alkyl) and —S(O)₂—O—(C₃-C₁₀ cycloalkyl), —S(O)₂—O—(CH₂)ₜ(C₆-C₁₀ aryl), —S(O)₂—O—(CH₂)ₜ(5-10 membered heteroaryl), —S(O)₂—O—(CH₂)ₜ(C₃-C₁₀ cycloalkyl), and —S(O)₂—O—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Thiol’ refers to the group —SH.

‘Aminocarbonylamino’ refers to the group —NR⁹⁶C(O)NR⁹⁶R⁹⁶ where each R⁹⁶ is independently hydrogen C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl, as defined herein; or where two R⁹⁶ groups, when attached to the same N, are joined to form an alkylene group.

‘Bicycloaryl’ refers to a monovalent aromatic hydrocarbon group derived by the removal of one hydrogen atom from a single carbon atom of a parent bicycloaromatic ring system. Typical bicycloaryl groups include, but are not limited to, groups derived from indane, indene, naphthalene, tetrahydronaphthalene, and the like. Particularly, an aryl group comprises from 8 to 11 carbon atoms.

‘Bicycloheteroaryl’ refers to a monovalent bicycloheteroaromatic group derived by the removal of one hydrogen atom from a single atom of a parent bicycloheteroaromatic ring system. Typical bicycloheteroaryl groups include, but are not limited to, groups derived from benzofuran, benzimidazole, benzindazole, benzdioxane, chromene, chromane, cinnoline, phthalazine, indole, indoline, indolizine, isobenzofuran, isochromene, isoindole, isoindoline, isoquinoline, benzothiazole, benzoxazole, naphthyridine, benzoxadiazole, pteridine, purine, benzopyran, benzpyrazine, pyridopyrimidine, quinazoline, quinoline, quinolizine, quinoxaline, benzomorphan, tetrahydroisoquinoline, tetrahydroquinoline, and the like. Preferably, the bicycloheteroaryl group is between 9-11 membered bicycloheteroaryl, with 5-10 membered heteroaryl being particularly preferred. Particular bicycloheteroaryl groups are those derived from benzothiophene, benzofuran, benzothiazole, indole, quinoline, isoquinoline, benzimidazole, benzoxazole and benzdioxane.

‘Compounds of the present invention’, and equivalent expressions, are meant to embrace the compounds as hereinbefore described, in particular compounds according to any of the formulae herein recited and/or described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

‘Cycloalkylalkyl’ refers to a radical in which a cycloalkyl group is substituted for a hydrogen atom of an alkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

‘Heterocycloalkylalkyl’ refers to a radical in which a heterocycloalkyl group is substituted for a hydrogen atom of an alkyl group. Typical heterocycloalkylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

‘Cycloalkenyl’ refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

‘Substituted cycloalkenyl’ refers to those groups recited in the definition of “substituted” herein, and particularly refers to a cycloalkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

‘Fused Cycloalkenyl’ refers to a cycloalkenyl having two of its ring carbon atoms in common with a second aliphatic or aromatic ring and having its olefinic unsaturation located to impart aromaticity to the cycloalkenyl ring.

‘Ethenyl’ refers to substituted or unsubstituted —C═C)—.

‘Ethylene’ refers to substituted or unsubstituted —(C—C)—.

‘Ethynyl’ refers to —(C≡C)—.

‘Hydrogen bond donor’ group refers to a group containing O—H, or N—H functionality. Examples of ‘hydrogen bond donor’ groups include —OH, —NH₂, and —NH—R⁹⁷ and wherein R⁹⁷ is alkyl, acyl, cycloalkyl, aryl, or heteroaryl.

‘Dihydroxyphosphoryl’ refers to the radical —PO(OH)₂.

‘Substituted dihydroxyphosphoryl’ refers to those groups recited in the definition of “substituted” herein, and particularly refers to a dihydroxyphosphoryl radical wherein one or both of the hydroxyl groups are substituted. Suitable substituents are described in detail below.

‘Aminohydroxyphosphoryl’ refers to the radical —PO(OH)NH₂.

‘Substituted aminohydroxyphosphoryl’ refers to those groups recited in the definition of “substituted” herein, and particularly refers to an aminohydroxyphosphoryl wherein the amino group is substituted with one or two substituents. Suitable substituents are described in detail below. In certain embodiments, the hydroxyl group can also be substituted.

‘Nitrogen-Containing Heterocycloalkyl’ group means a 4 to 7 membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

‘Thioketo’ refers to the group ═S.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Prodrugs’ refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

‘Solvate’ refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans. The terms ‘human’, ‘patient’ and ‘subject’ are used interchangeably herein.

‘Therapeutically effective amount’ means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

‘Preventing’ or ‘prevention’ refers to a reduction in risk of acquiring or developing a disease or disorder (i.e., causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e., arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

‘Compounds of the present invention’, and equivalent expressions, are meant to embrace compounds of the Formula(e) as hereinbefore described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

When ranges are referred to herein, for example but without limitation, C₁-C₈ alkyl, the citation of a range should be considered a representation of each member of said range.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. Particularly the C₁ to C₈ alkyl, C₂-C₈ alkenyl, aryl, C₇-C₁₂ substituted aryl, and C₇-C₁₂ arylalkyl esters of the compounds of the invention.

As used herein, the term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure S-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### The Compounds

The present invention provides a method for preventing, treating or ameliorating in a mammal a disease or condition that is causally related to the aberrant activity of the Wnt signaling pathway in vivo, which comprises administering to the mammal an effective disease-treating or condition-treating amount of a compound according to formula IA:

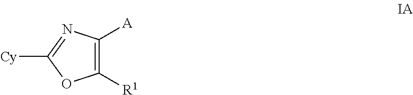

wherein
A is -L¹-X-L²—C(O)—NR^{2a}R^{2b} or -L¹-X-L²—C(O)—OR^{2a};
Cy is substituted or unsubstituted aryl or substituted or unsubstituted heteroaryl;
each of L¹ and L² is independently substituted or unsubstituted C₁-C₇ alkylene or heteroalkylene;
R¹ is hydrogen, halo, or substituted or unsubstituted C₁-C₆ alkyl;
each R^{2a} and R^{2b} is independently selected from H, substituted or unsubstituted alkyl, substituted or unsubstituted cycloalkyl, substituted or unsubstituted aryl, and substituted or unsubstituted heteroaryl; or R^{2a} and R^{2b} are joined together to form a heterocycloalkyl or heteroaryl ring;
X is —O—; or X is S, SO or SO₂;
provided that when the compound is according to formula I, X is S, SO or SO₂, then R^{2a} is H and R^{2b} is ethylene substituted with fluorophenyl, trifluorophenyl, or pyridyl;
or a pharmaceutically acceptable salt, solvate or prodrug thereof;
and stereoisomers, isotopic variants and tautomers thereof.

In certain aspects, the present invention provides a composition of a compound according to formula IA.

In one embodiment, A is -L¹-X-L²-C(O)—NR^{2a}R^{2b}. In another embodiment, A is -L¹-X-L²-C(O)—OR^{2a}.

In certain aspects, the present invention provides a composition of a compound according to formula I:

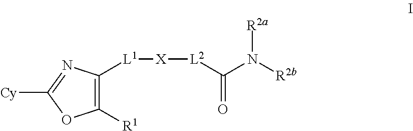

wherein
Cy is substituted or unsubstituted aryl or substituted or unsubstituted heteroaryl;
each of L¹ and L² is independently substituted or unsubstituted C₁-C₇ alkylene or heteroalkylene;
R¹ is hydrogen, halo, or substituted or unsubstituted C₁-C₆ alkyl;
each R^{2a} and R^{2b} is independently selected from H, substituted or unsubstituted alkyl, substituted or unsubstituted cycloalkyl, substituted or unsubstituted aryl, and substituted or unsubstituted heteroaryl; or R^{2a} and R^{2b} are joined together to form a heterocycloalkyl or heteroaryl ring;
X is —O—; or X is S, SO or SO₂;
provided that when X is S, SO or SO₂, then R^{2a} is H and R^{2b} is ethylene substituted with fluorophenyl, trifluorophenyl, or pyridyl;
or a pharmaceutically acceptable salt, solvate or prodrug thereof;
and stereoisomers, isotopic variants and tautomers thereof.

In certain aspects, the present invention provides a composition of a compound according to formula I′:

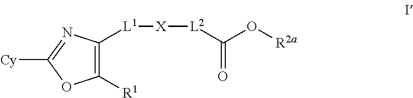

wherein
Cy is substituted or unsubstituted aryl or substituted or unsubstituted heteroaryl;
each of L¹ and L² is independently substituted or unsubstituted C₁-C₇ alkylene or heteroalkylene;
R¹ is halo, or substituted or unsubstituted C₁-C₆ alkyl;
R^{2a} is selected from H, substituted or unsubstituted alkyl, substituted or unsubstituted cycloalkyl, substituted or unsubstituted aryl, and substituted or unsubstituted heteroaryl;
X is —O—; or X is S, SO or SO₂;
or a pharmaceutically acceptable salt, solvate or prodrug thereof;
   and stereoisomers, isotopic variants and tautomers thereof.

In one embodiment, L¹ is methylene, ethylene, propylene, or butylene, each of which unsubstituted or substituted with one or more groups selected from C₁-C₄ alkyl, halo, and hydroxyl.

In another embodiment, L¹ is —CH₂—, —CH₂—CH₂—, —CH₂—CH₂—CH₂—, or —CH₂—CH₂—CH₂—CH₂—.

In one particular embodiment, L¹ is —CH₂—.

In one embodiment, L² is methylene, ethylene, propylene, or butylene, each of which unsubstituted or substituted with one or more groups selected from C₁-C₄ alkyl, halo, and hydroxyl.

In another embodiment, L² is —CH₂—, —CH₂—CH₂—, —CH₂—CH₂—CH₂—, or —CH₂—CH₂—CH₂—CH₂—.

In one particular embodiment, L² is —CH₂—.

In one particular embodiment, Cy is substituted or unsubstituted aryl.

In another embodiment, Cy is substituted or unsubstituted phenyl.

In another embodiment, Cy is substituted or unsubstituted naphthyl.

In another embodiment, Cy is substituted or unsubstituted heteroaryl.

In another embodiment, Cy is substituted or unsubstituted pyridyl.

In another embodiment, Cy is substituted or unsubstituted pyrimidinyl.

In one embodiment, X is —O—.

In one particular embodiment, with respect to compounds of formula IA, the compound is according to formula II:

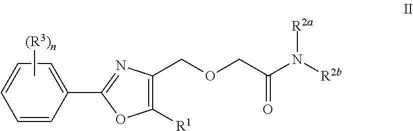

and wherein R¹, R^{2a}, and R^{2b} are as described for formula IA; n is 1, 2, 3, 4, or 5; and each R³ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

In another particular embodiment, with respect to compounds of formula IA, the compound is according to formula II′ or II″:

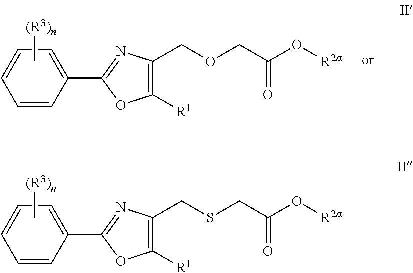

and wherein R¹, and R^{2a} are as described for formula IA; n is 1, 2, 3, 4, or 5; and each R³ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

In one embodiment, R¹ is substituted or unsubstituted alkyl or halo.

In another embodiment, R¹ is H or substituted or unsubstituted C₁-C₆ alkyl.

In another embodiment, R¹ is halo.

In another embodiment, R¹ is Me, Et, i-Pr, n-Pr, n-Bu, F, Cl, Br, or I.

In a particular embodiment, R¹ is Me.

In one particular embodiment, with respect to compounds of formula IA, the compound is according to formula III:

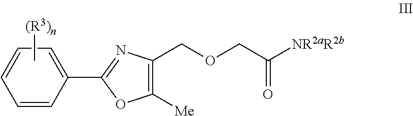

wherein R^{2a} and R^{2b} are as described for formula I; and n and R³ are as described for formula II.

In one particular embodiment, with respect to compounds of formula IA, the compound is according to formula III′ or III″:

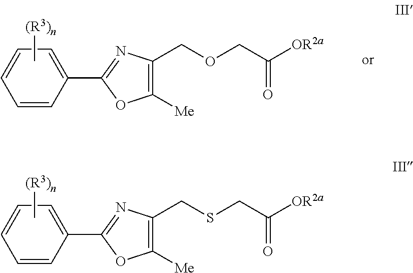

wherein R^{2a} is as described for formula IA; and n and R³ are as described for formula II′.

In one embodiment, each of R³ is H.

In another embodiment, n is 1; and R³ is alkyl, alkoxy, haloalkyl, or halo.

In another embodiment, n is 1 or 2; and R³ is Me, Et, i-Pr, OMe, OEt, O-i-Pr, Cl, or F.

In another embodiment, n is 1 or 2; and R³ is Me, OMe, SMe, or Et.

In another embodiment, n is 1; and R³ is Me.

In another embodiment, n is 1; and R³ is Et.

In one embodiment, R^{2a} is H.

In another embodiment, R^{2a} is substituted or unsubstituted alkyl.

In another embodiment, R^{2a} is substituted or unsubstituted benzyl.

In another embodiment, R^{2a} is substituted or unsubstituted phenethyl.

In one particular embodiment, R^{2a} is substituted or unsubstituted cycloalkyl.

In another embodiment, R^{2a} is cyclopropyl.

In one embodiment, R^{2b} is substituted or unsubstituted heteroaryl.

In another embodiment, R^{2b} is substituted or unsubstituted heterocycloalkyl.

In another embodiment, each of R^{2a} and R^{2b} is H.

In another embodiment, one of R^{2a} and R^{2b} is substituted or unsubstituted alkyl and the other is H.

In another embodiment, one of R^{2a} and R^{2b} is alkyl substituted with aryl, heteroaryl, cycloalkyl or heterocycloalkyl; and the other is H.

In another embodiment, one of R^{2a} and R^{2b} is methyl, ethyl, or n-propyl substituted with phenyl, pyridyl, cycloalkyl or heterocycloalkyl; and the other is H.

In another embodiment, one of R^{2a} and R^{2b} is methyl, ethyl, or n-propyl substituted with phenyl, pyridyl, cyclopropyl, cyclohexyl, cyclopentyl, cyclobutyl, piperidinyl, morphlinyl or piperazinyl; and the other is H.

In another embodiment, one of R^{2a} and R^{2b} is ethyl substituted with phenyl, pyridyl, cyclopropyl, cyclohexyl, cyclopentyl, cyclobutyl, piperidinyl, morphlinyl or piperazinyl; and the other is H.

In another embodiment, one of R^{2a} and R^{2b} is phenylethylene, pyridylethylene, cyclopropylethylene, cyclohexylethylene, cyclopentylethylene, cyclobutylethylene, piperidinylethylene, morphlinylethylene, or piperazinylethylene; and the other is H.

In one embodiment, each of phenyl, pyridyl, cyclopropyl, cyclohexyl, cyclopentyl, cyclobutyl, piperidinyl, morphlinyl or piperazinyl is substituted or unsubstituted.

In one embodiment, each of phenyl, pyridyl, cyclopropyl, cyclohexyl, cyclopentyl, cyclobutyl, piperidinyl, morphlinyl or piperazinyl is substituted with alkyl, halo or CN.

In another embodiment, one of R^{2a} and R^{2b} is substituted or unsubstituted benzyl and the other is H.

In another embodiment, one of R^{2a} and R^{2b} is substituted or unsubstituted phenethyl and the other is H.

In another embodiment, one of R^{2a} and R^{2b} is substituted or unsubstituted cycloalkyl and the other is H.

In another embodiment, one of R^{2a} and R^{2b} is substituted or unsubstituted cyclopropyl and the other is H.

In another embodiment, one of R^{2a} and R^{2b} is substituted or unsubstituted cyclopentyl or cyclobutyl and the other is H.

In another embodiment, R^{2a} and R^{2b} join together to form a heterocycloalkyl or heteroaryl ring.

In another embodiment, NR^{2a}R^{2b} is:

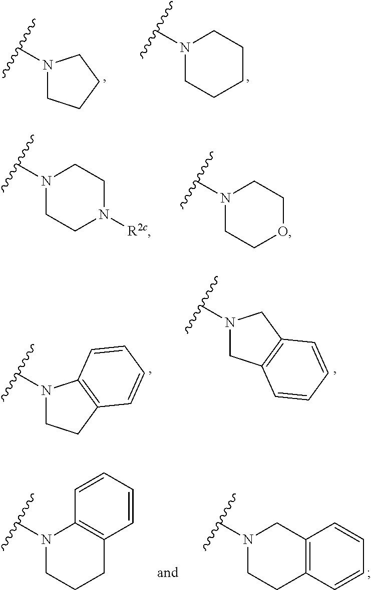

and wherein R^{2c} is H or alkyl.

In one particular embodiment, with respect to compounds of formula IA, the compound is according to formula IVa, IVb, IVc or IVd:

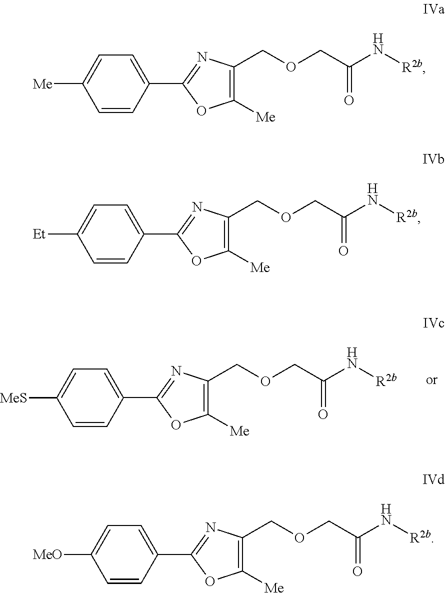

wherein R^{2b} is as described for formula IA.

In another embodiment, R^{2b} is substituted or unsubstituted cycloalkyl, substituted or unsubstituted phenyl, substituted or unsubstituted benzyl, or substituted or unsubstituted phenethyl.

In another embodiment, R^{2b} is substituted or unsubstituted heteroaryl, or substituted or unsubstituted heterocycloalkyl.

In another embodiment, R^{2b} is H.

In one particular embodiment, with respect to compounds of formula IA, the compound is according to formula IVa′, IVb′, IVc′ or IVd′:

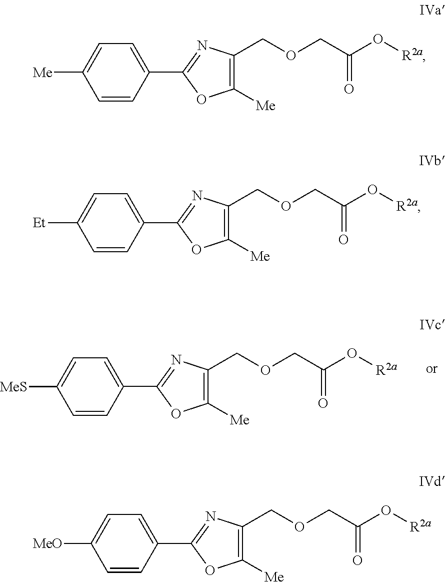

wherein R^{2A} is as described for formula IA.

In another embodiment, R^{2a} is H, substituted or unsubstituted cycloalkyl, substituted or unsubstituted phenyl, substituted or unsubstituted benzyl, or substituted or unsubstituted phenethyl.

In another embodiment, R^{2a} is substituted or unsubstituted heteroaryl, or substituted or unsubstituted heterocycloalkyl.

In another embodiment, R^{2a} is H, Me, Et, n-Pr, i-Pr, n-Bu, i-Bu, sec-Bu, t-Bu, or cyclopropyl.

In one particular embodiment, with respect to compounds of formula IA, the compound is according to formula Va, Vb, Vc, or Vd:

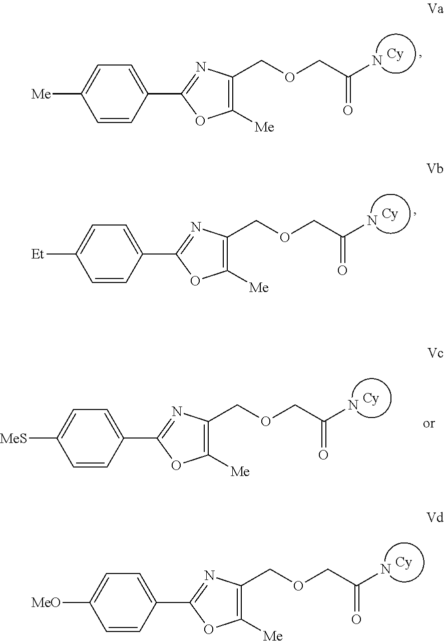

or a pharmaceutically acceptable salt, solvate or prodrug thereof;
and stereoisomers, isotopic variants and tautomers thereof;
wherein Cy is

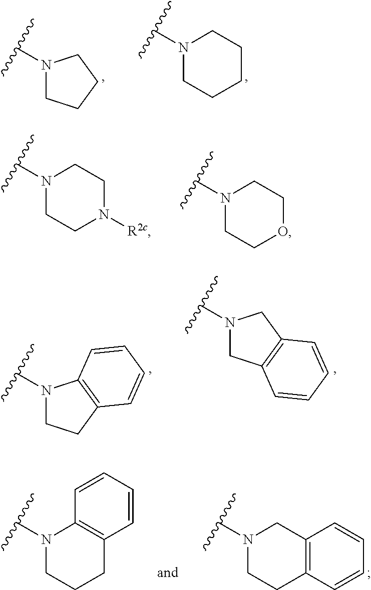

and wherein R^{2c} is H or alkyl.

In one particular embodiment, with respect to compounds of formula IA, the compound is according to formula VIa, VIb, VIc or VId:

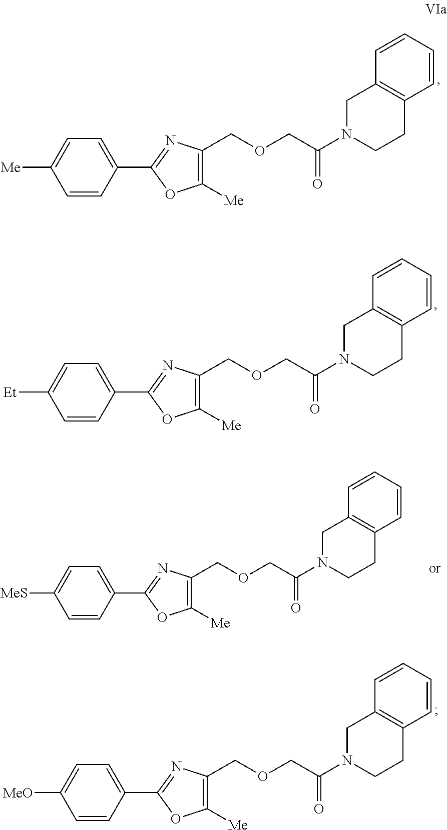 [SMILES: Cc1ccc(-c2nc(COCC(=O)N3CCc4ccccc4C3)c(C)o2)cc1 ; CCc1ccc(-c2nc(COCC(=O)N3CCc4ccccc4C3)c(C)o2)cc1 ; CSc1ccc(-c2nc(COCC(=O)N3CCc4ccccc4C3)c(C)o2)cc1 ; COc1ccc(-c2nc(COCC(=O)N3CCc4ccccc4C3)c(C)o2)cc1]

or a pharmaceutically acceptable salt, solvate or prodrug thereof;
   and stereoisomers, isotopic variants and tautomers thereof.

In one particular embodiment, with respect to compounds of formula IA, the compound is according to formula VIIa, VIIb, VIIc or VIId:

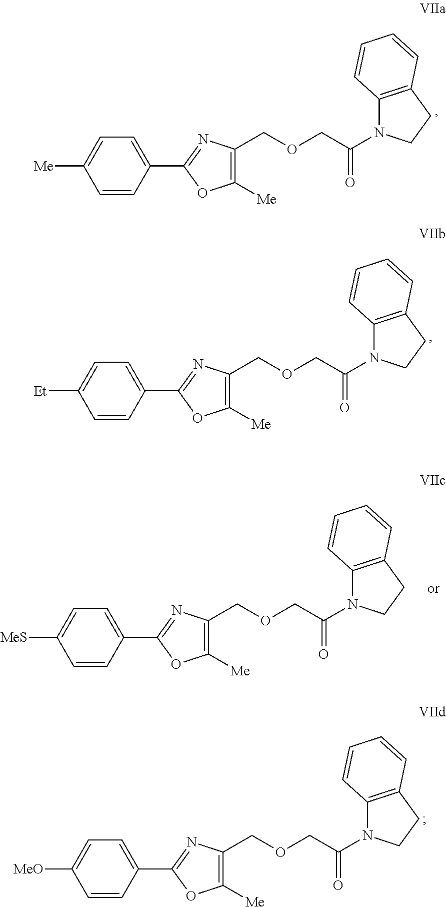 [SMILES: Cc1ccc(-c2nc(COCC(=O)N3CCc4ccccc43)c(C)o2)cc1 ; CCc1ccc(-c2nc(COCC(=O)N3CCc4ccccc43)c(C)o2)cc1 ; CSc1ccc(-c2nc(COCC(=O)N3CCc4ccccc43)c(C)o2)cc1 ; COc1ccc(-c2nc(COCC(=O)N3CCc4ccccc43)c(C)o2)cc1]

or a pharmaceutically acceptable salt, solvate or prodrug thereof;
and stereoisomers, isotopic variants and tautomers thereof.

In one particular embodiment, with respect to compounds of formula IA, the compound is according to formula VIIIa, VIIIb, VIIIc or VIIId:

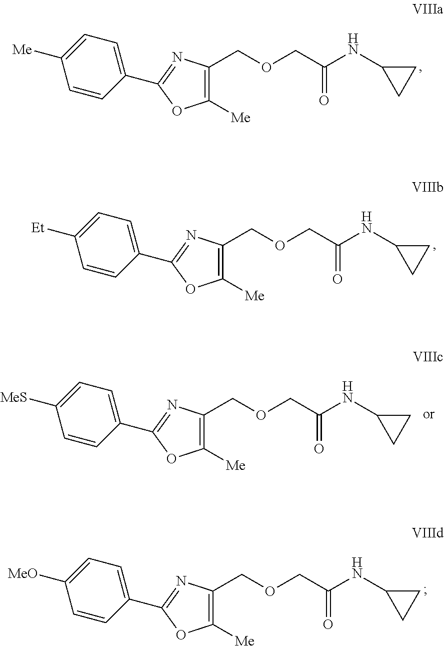 [SMILES: Cc1ccc(-c2nc(COCC(=O)NC3CC3)c(C)o2)cc1 ; CCc1ccc(-c2nc(COCC(=O)NC3CC3)c(C)o2)cc1 ; CSc1ccc(-c2nc(COCC(=O)NC3CC3)c(C)o2)cc1 ; COc1ccc(-c2nc(COCC(=O)NC3CC3)c(C)o2)cc1]

or a pharmaceutically acceptable salt, solvate or prodrug thereof;
   and stereoisomers, isotopic variants and tautomers thereof.

In one particular embodiment, with respect to compounds of formula IA, the compound is according to formula IXa, IXb, IXc or IXd:

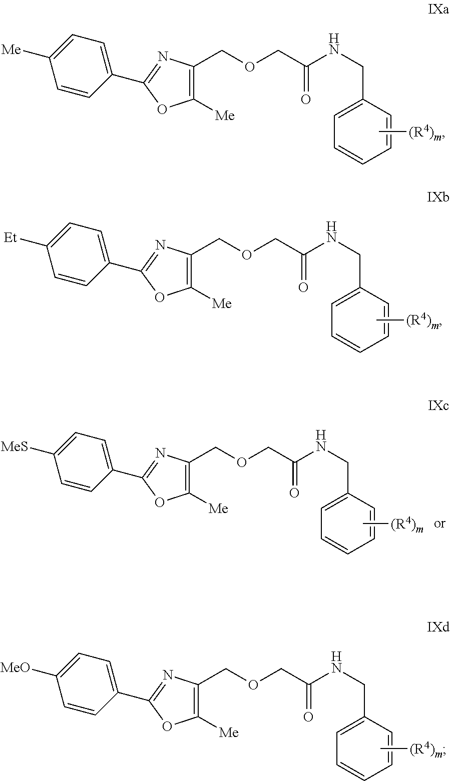

or a pharmaceutically acceptable salt, solvate or prodrug thereof;
and stereoisomers, isotopic variants and tautomers thereof;
   wherein m is 1, 2, 3, 4, or 5; and
   each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

In one particular embodiment, with respect to compounds of formula IA, the compound is according to formula Xa, Xb, Xc or Xd:

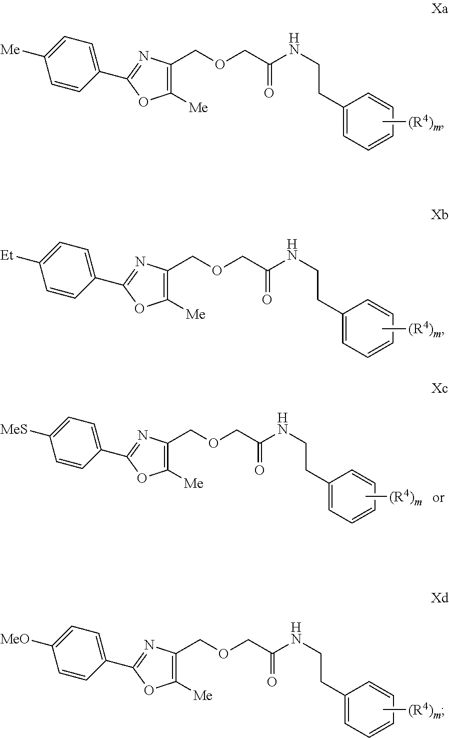

or a pharmaceutically acceptable salt, solvate or prodrug thereof;
and stereoisomers, isotopic variants and tautomers thereof;
wherein m is 1, 2, 3, 4, or 5; and
each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

In one embodiment, m is 1 or 2; and each R⁴ is independently Me, Et, i-Pr, OMe, OEt, O-i-Pr, Cl, or F.

In another embodiment, each R⁴ is H.

In another embodiment, m is 1; and R⁴ is Me, Et, i-Pr, OMe, OEt, O-i-Pr, Cl, or F.

In another embodiment, m is 1; and R⁴ is 4-Cl, or 4-F.

In one particular embodiment, with respect to compounds of formula IA, the compound is according to formula XIa, XIb, XIc or XId:

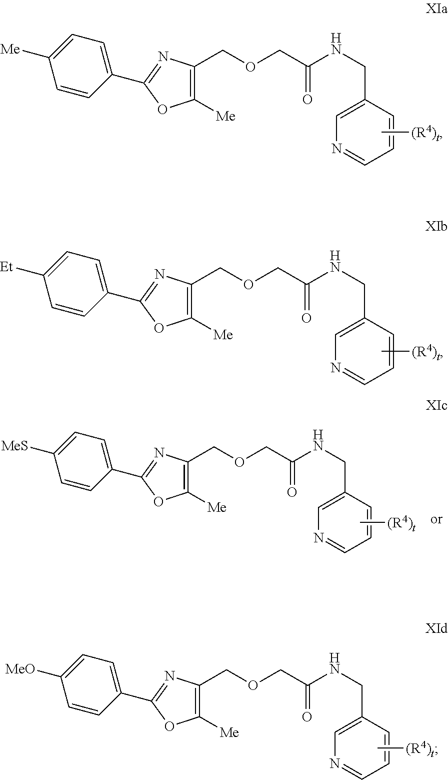

or a pharmaceutically acceptable salt, solvate or prodrug thereof;
and stereoisomers, isotopic variants and tautomers thereof;
   wherein t is 1, 2, 3, 4, or 5; and
   each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

In one particular embodiment, with respect to compounds of formula IA, the compound is according to formula XIIa, XIIb, XIII or XIId:

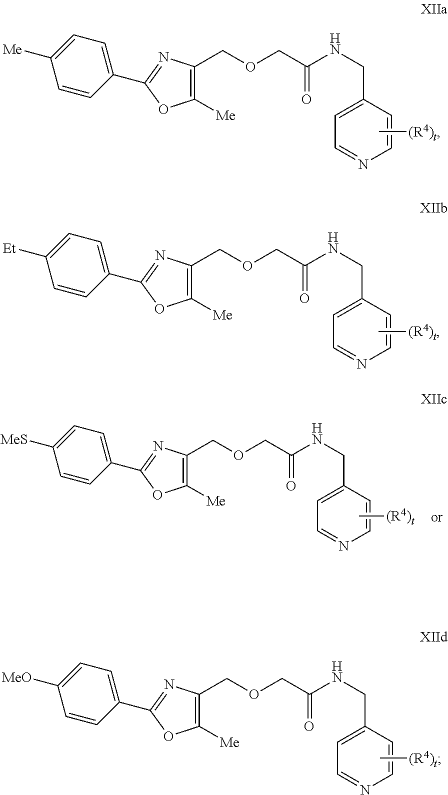

or a pharmaceutically acceptable salt, solvate or prodrug thereof;
and stereoisomers, isotopic variants and tautomers thereof;
   wherein t is 1, 2, 3, 4, or 5; and
   each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

In one particular embodiment, with respect to compounds of formula IA, the compound is according to formula XIIIa, XIIIb, XIIIc or XIIId:

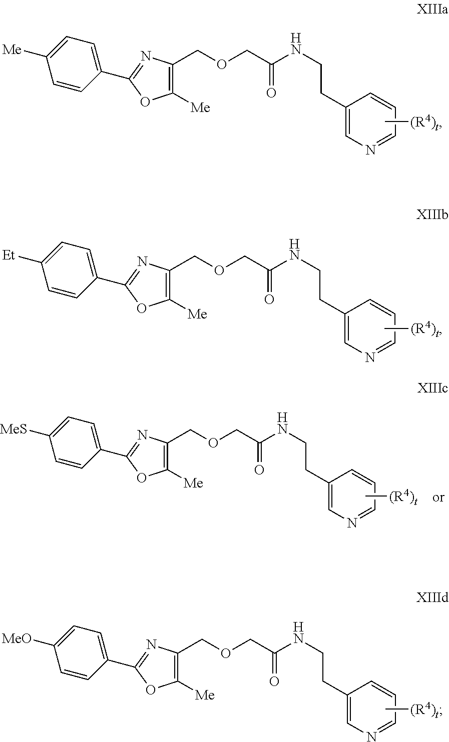

or a pharmaceutically acceptable salt, solvate or prodrug thereof;
and stereoisomers, isotopic variants and tautomers thereof;
   wherein t is 1, 2, 3, 4, or 5; and
   each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

In one particular embodiment, with respect to compounds of formula IA, the compound is according to formula XIVa, XIVb, XIVc or XIVd:

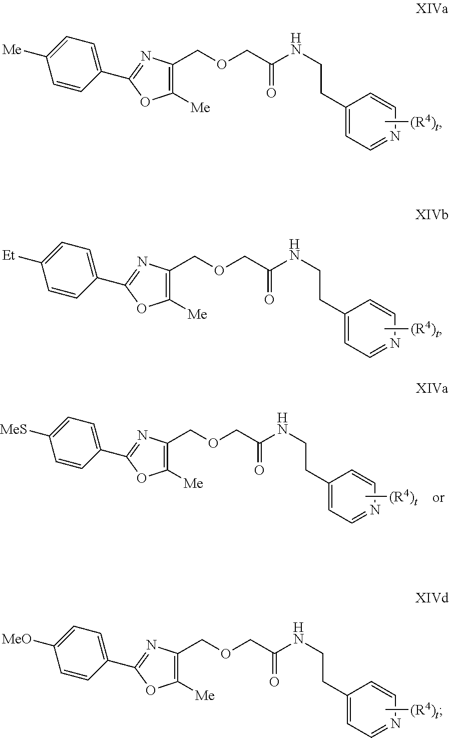

or a pharmaceutically acceptable salt, solvate or prodrug thereof;
and stereoisomers, isotopic variants and tautomers thereof;
   wherein t is 1, 2, 3, 4, or 5; and
   each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

In one embodiment, t is 1 or 2; and each R⁴ is independently Me, Et, i-Pr, OMe, OEt, O-i-Pr, Cl, or F.

In another embodiment, each R⁴ is H.

In another embodiment, t is 1; and R⁴ is Me, Et, i-Pr, OMe, OEt, O-i-Pr, Cl, or F.

In one particular embodiment, with respect to compounds of formula IA, the compound is according to formula XVa, XVb, XVc or XVd:

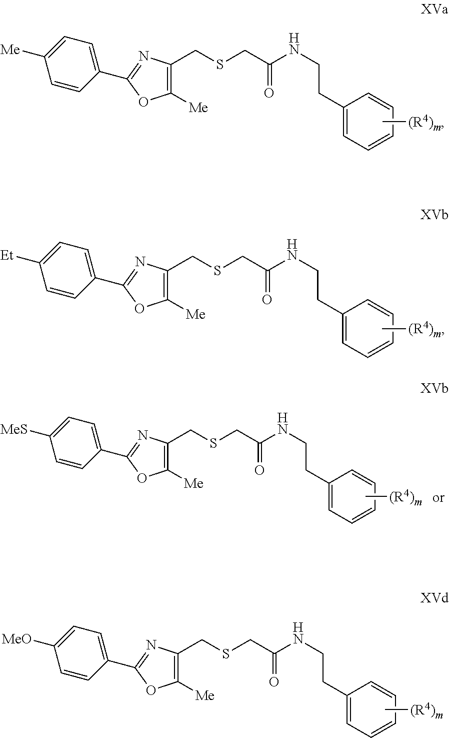

or a pharmaceutically acceptable salt, solvate or prodrug thereof;
and stereoisomers, isotopic variants and tautomers thereof;
wherein m is 1, 2, 3, 4, or 5; and
each R⁴ is independently F or CF₃.

In one particular embodiment, m is 1 and R⁴ is 4-F or 4-CF₃.

In one particular embodiment, with respect to compounds of formula IA, the compound is according to formula XVIa, XVIb, XVIc or XVId:

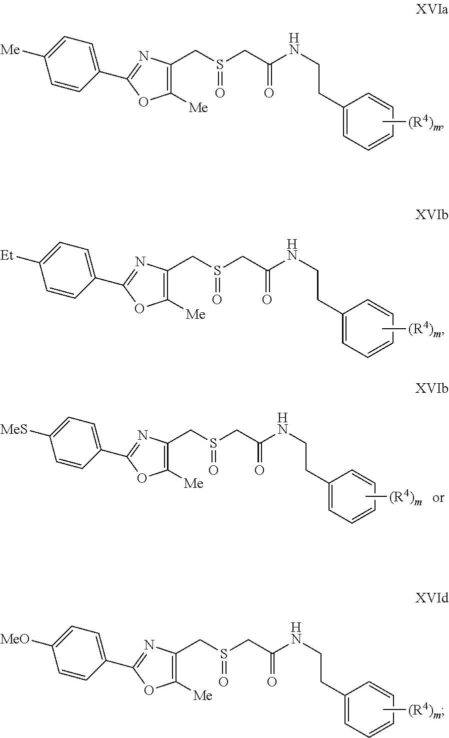

or a pharmaceutically acceptable salt, solvate or prodrug thereof;
and stereoisomers, isotopic variants and tautomers thereof;
wherein m is 1, 2, 3, 4, or 5; and
each R⁴ is independently F or CF₃.

In one particular embodiment, m is 1 and R⁴ is 4-F. In another particular embodiment, m is 1 and R⁴ is 4-CF₃.

In one particular embodiment, with respect to compounds of formula IA, the compound is according to formula XVIIa, XVIIb, XVIIc or XVIId:

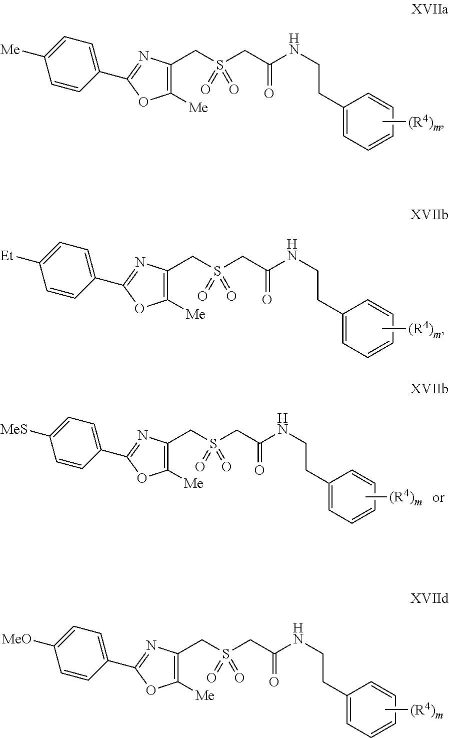

or a pharmaceutically acceptable salt, solvate or prodrug thereof;
and stereoisomers, isotopic variants and tautomers thereof;
wherein m is 1, 2, 3, 4, or 5; and
each R⁴ is independently F or CF₃.

In one particular embodiment, m is 1 and R⁴ is 4-F. In another particular embodiment, m is 1 and R⁴ is 4-CF₃.

In one particular embodiment, with respect to compounds of formula IA, the compound is according to formula XVIIIa, XVIIIb, XVIIIc or XVIIId:

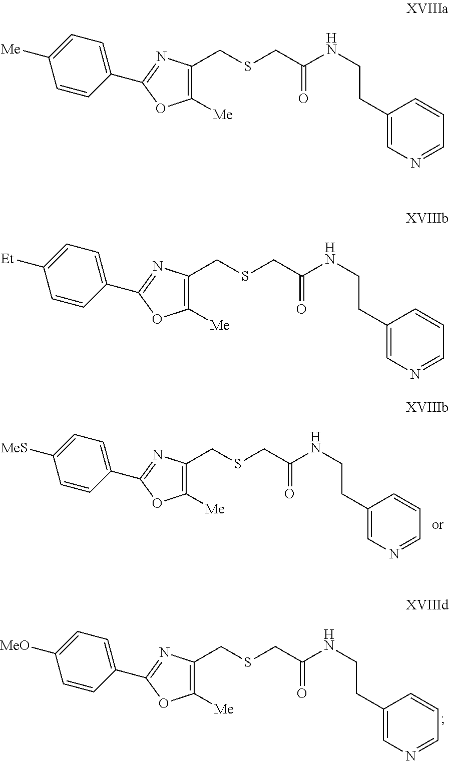 [SMILES: Cc1ccc(-c2nc(CSCC(=O)NCCc3cccnc3)c(C)o2)cc1 ; CCc1ccc(-c2nc(CSCC(=O)NCCc3cccnc3)c(C)o2)cc1 ; CSc1ccc(-c2nc(CSCC(=O)NCCc3cccnc3)c(C)o2)cc1 ; COc1ccc(-c2nc(CSCC(=O)NCCc3cccnc3)c(C)o2)cc1]

or a pharmaceutically acceptable salt, solvate or prodrug thereof;
and stereoisomers, isotopic variants and tautomers thereof.

In one particular embodiment, with respect to compounds of formula IA, the compound is according to formula XIXa, XIXb, XIXc or XIXd:

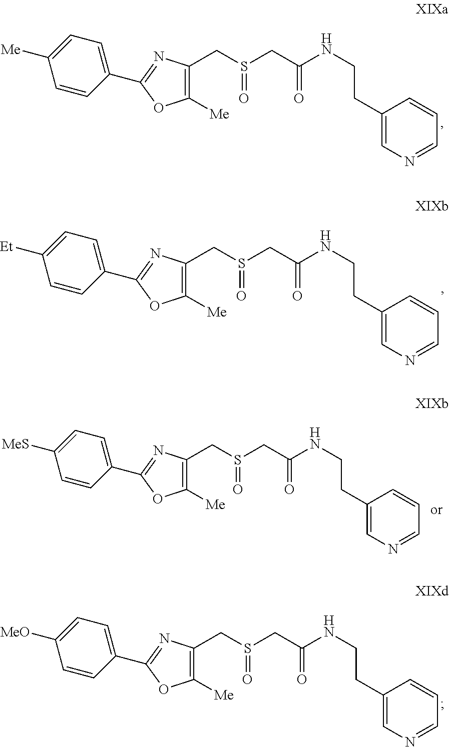 [SMILES: Cc1ccc(-c2nc(CS(=O)CC(=O)NCCc3cccnc3)c(C)o2)cc1 ; CCc1ccc(-c2nc(CS(=O)CC(=O)NCCc3cccnc3)c(C)o2)cc1 ; CSc1ccc(-c2nc(CS(=O)CC(=O)NCCc3cccnc3)c(C)o2)cc1 ; COc1ccc(-c2nc(CS(=O)CC(=O)NCCc3cccnc3)c(C)o2)cc1]

or a pharmaceutically acceptable salt, solvate or prodrug thereof;
and stereoisomers, isotopic variants and tautomers thereof.

In one particular embodiment, with respect to compounds of formula IA, the compound is according to formula XXa, XXb, XXc or XXd:

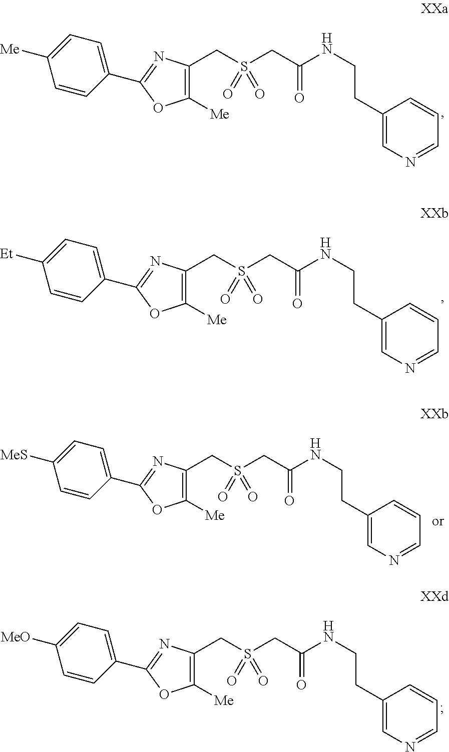 [SMILES: Cc1ccc(-c2nc(CS(=O)(=O)CC(=O)NCCc3cccnc3)c(C)o2)cc1 ; CCc1ccc(-c2nc(CS(=O)(=O)CC(=O)NCCc3cccnc3)c(C)o2)cc1 ; CSc1ccc(-c2nc(CS(=O)(=O)CC(=O)NCCc3cccnc3)c(C)o2)cc1 ; COc1ccc(-c2nc(CS(=O)(=O)CC(=O)NCCc3cccnc3)c(C)o2)cc1]

or a pharmaceutically acceptable salt, solvate or prodrug thereof;
and stereoisomers, isotopic variants and tautomers thereof.

In one particular embodiment, with respect to compounds of formula IA, the compound is according to formula XXIa, XXIb, XXIc or XXId:

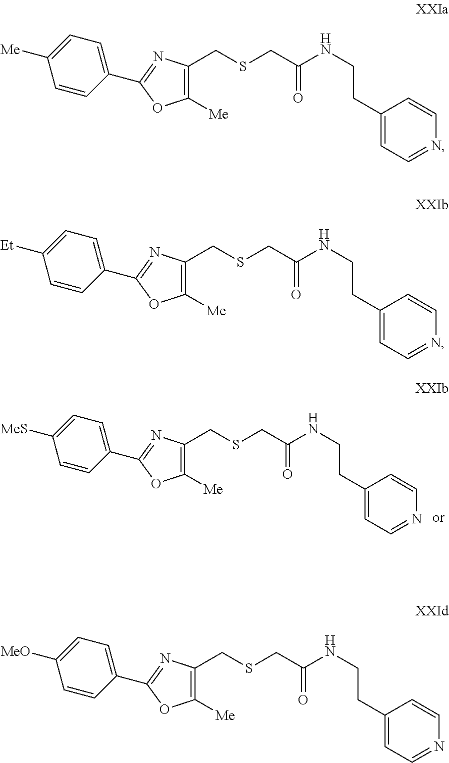 [SMILES: Cc1ccc(-c2nc(CSCC(=O)NCCc3ccncc3)c(C)o2)cc1 ; CCc1ccc(-c2nc(CSCC(=O)NCCc3ccncc3)c(C)o2)cc1 ; CSc1ccc(-c2nc(CSCC(=O)NCCc3ccncc3)c(C)o2)cc1 ; COc1ccc(-c2nc(CSCC(=O)NCCc3ccncc3)c(C)o2)cc1]

or a pharmaceutically acceptable salt, solvate or prodrug thereof;
and stereoisomers, isotopic variants and tautomers thereof.

In one particular embodiment, with respect to compounds of formula IA, the compound is according to formula XXIIa, XXIIb, XXIIc or XXIId:

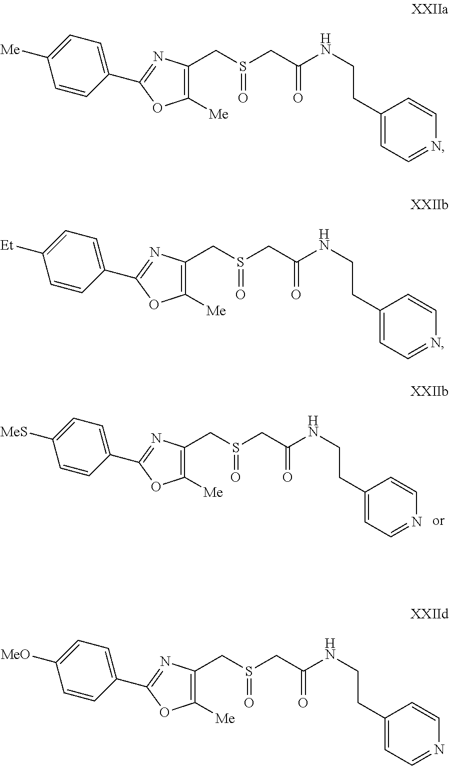 [SMILES: Cc1ccc(-c2nc(CS(=O)CC(=O)NCCc3ccncc3)c(C)o2)cc1 ; CCc1ccc(-c2nc(CS(=O)CC(=O)NCCc3ccncc3)c(C)o2)cc1 ; CSc1ccc(-c2nc(CS(=O)CC(=O)NCCc3ccncc3)c(C)o2)cc1 ; COc1ccc(-c2nc(CS(=O)CC(=O)NCCc3ccncc3)c(C)o2)cc1]

or a pharmaceutically acceptable salt, solvate or prodrug thereof;
and stereoisomers, isotopic variants and tautomers thereof.

In one particular embodiment, with respect to compounds of formula IA, the compound is according to formula XXIIIa, XXIIIb, XXIIIc or XXIIId:

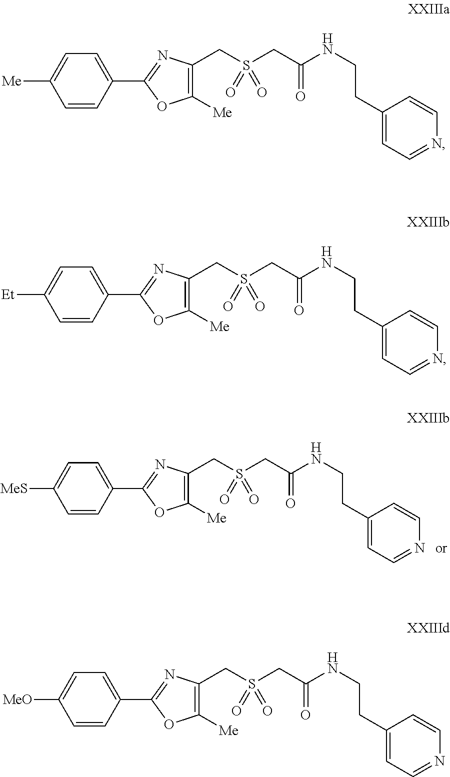 [SMILES: Cc1ccc(-c2nc(CS(=O)(=O)CC(=O)NCCc3ccncc3)c(C)o2)cc1 ; CCc1ccc(-c2nc(CS(=O)(=O)CC(=O)NCCc3ccncc3)c(C)o2)cc1 ; CSc1ccc(-c2nc(CS(=O)(=O)CC(=O)NCCc3ccncc3)c(C)o2)cc1 ; COc1ccc(-c2nc(CS(=O)(=O)CC(=O)NCCc3ccncc3)c(C)o2)cc1]

or a pharmaceutically acceptable salt, solvate or prodrug thereof;
and stereoisomers, isotopic variants and tautomers thereof.

In one particular embodiment, with respect to compounds of formula IA, the compound is according to formula XXIVa′, XXIVb′, XXIVc′ or XXIVd′:

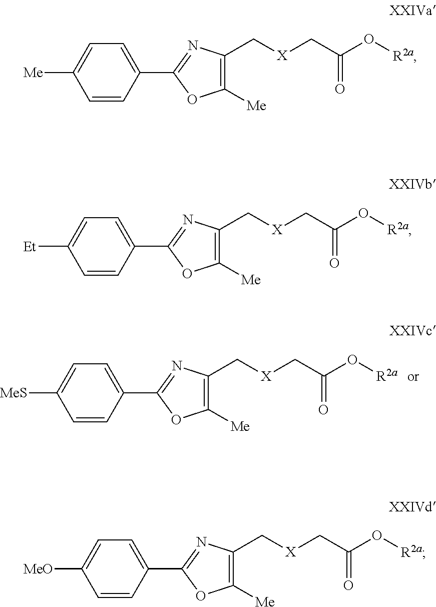

or a pharmaceutically acceptable salt, solvate or prodrug thereof;
and stereoisomers, isotopic variants and tautomers thereof;
wherein R^{2a} is as described herein; and X is S, S(O), or S(O)₂.

In one embodiment, X is S. In another embodiment, X is S(O). In another embodiment, X is S(O)₂.

In one particular embodiment, with respect to compounds of formula IA, the compound is according to formula XXVa′, XXVb′, XXVc′ or XXVd′:

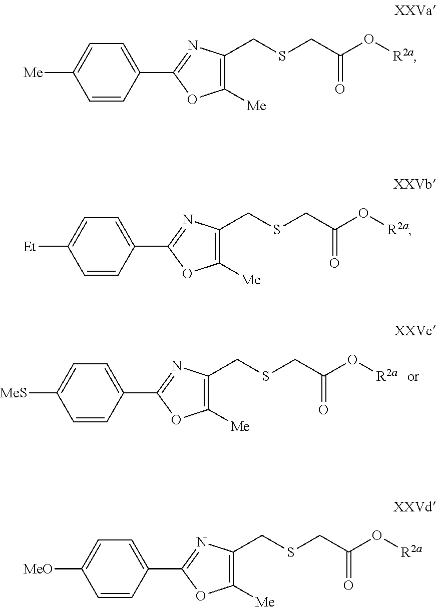

or a pharmaceutically acceptable salt, solvate or prodrug thereof;
   and stereoisomers, isotopic variants and tautomers thereof;
   wherein R^{2a} is as described herein.

In one embodiment, R^{2a} is H, substituted or unsubstituted cycloalkyl, substituted or unsubstituted phenyl, substituted or unsubstituted benzyl, or substituted or unsubstituted phenethyl. In another embodiment, R^{2a} is substituted or unsubstituted heteroaryl, or substituted or unsubstituted heterocycloalkyl. In another embodiment, R^{2a} is R^{2a} is H, Me, Et, n-Pr, i-Pr, n-Bu, i-Bu, sec-Bu, t-Bu, or cyclopropyl. In another embodiment, R^{2a} is H.

In one particular embodiment, with respect to compounds of formula IA, the compound is according to formula XXVIa, XXVIb, XXVIc or XXVId:

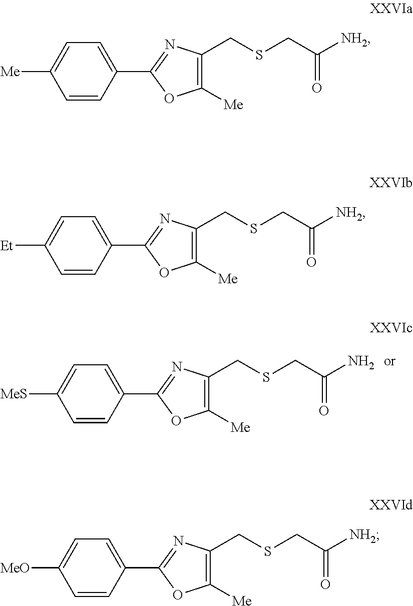 [SMILES: Cc1ccc(-c2nc(CSCC(N)=O)c(C)o2)cc1 ; CCc1ccc(-c2nc(CSCC(N)=O)c(C)o2)cc1 ; CSc1ccc(-c2nc(CSCC(N)=O)c(C)o2)cc1 ; COc1ccc(-c2nc(CSCC(N)=O)c(C)o2)cc1]

or a pharmaceutically acceptable salt, solvate or prodrug thereof;
and stereoisomers, isotopic variants and tautomers thereof.

In one particular embodiment, with respect to compounds of formula IA, the compound is according to formula XXVIa′, XXVIb′, XXVIc′ or XXVId′:

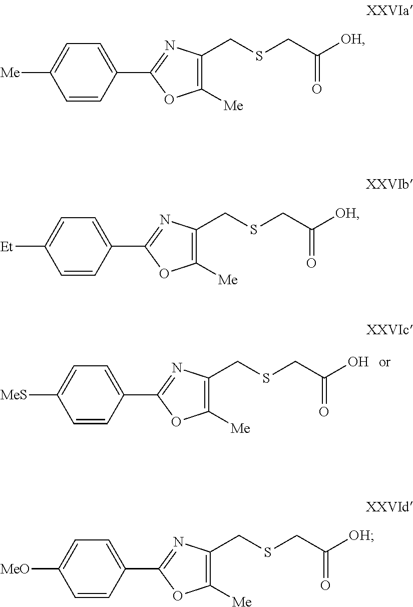 [SMILES: Cc1ccc(-c2nc(CSCC(=O)O)c(C)o2)cc1 ; CCc1ccc(-c2nc(CSCC(=O)O)c(C)o2)cc1 ; CSc1ccc(-c2nc(CSCC(=O)O)c(C)o2)cc1 ; COc1ccc(-c2nc(CSCC(=O)O)c(C)o2)cc1]

or a pharmaceutically acceptable salt, solvate or prodrug thereof;
and stereoisomers, isotopic variants and tautomers thereof.

In a particular aspect, the present invention provides a composition of a compound according to formula I-XXVId.

In another particular aspect, the present invention provides a composition of a compound according to formula XVa-XXVId.

In one particular embodiment, with respect to compounds of formula I, the compound is selected from Table 1.

In one particular embodiment, with respect to compounds of formula I, the compound is selected from Table 2.

In certain aspects, the present invention provides prodrugs and derivatives of the compounds according to the formulae above. Prodrugs are derivatives of the compounds of the invention, which have metabolically cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds of the invention.

### Pharmaceutical Compositions

When employed as pharmaceuticals, the compounds of this invention are typically administered in the form of a pharmaceutical composition. Such compositions can be prepared in a manner well known in the pharmaceutical art and comprise at least one active compound.

Generally, the compounds of this invention are administered in a pharmaceutically effective amount. The amount of the compound actually administered will typically be determined by a physician, in the light of the relevant circumstances, including the condition to be treated, the chosen route of administration, the actual compound—administered, the age, weight, and response of the individual patient, the severity of the patient's symptoms, and the like.

The pharmaceutical compositions of this invention can be administered by a variety of routes including oral, rectal, transdermal, subcutaneous, intravenous, intramuscular, and intranasal. Depending on the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

A compound of the invention may be admixed as a dry powder with a dry gelatin binder in an approximate 1:2 weight ratio. A minor amount of magnesium stearate is added as a lubricant. The mixture is formed into 240-270 mg tablets (80-90 mg of active amide compound per tablet) in a tablet press.

### Formulation 2—Capsules

A compound of the invention may be admixed as a dry powder with a starch diluent in an approximate 1:1 weight ratio. The mixture is filled into 250 mg capsules (125 mg of active amide compound per capsule).

### Formulation 3—Liquid

A compound of the invention (125 mg), sucrose (1.75 g) and xanthan gum (4 mg) may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color would then be diluted with water and added with stirring. Sufficient water is then added to produce a total volume of 5 mL.

### Formulation 4—Tablets

A compound of the invention may be admixed as a dry powder with a dry gelatin binder in an approximate 1:2 weight ratio. A minor amount of magnesium stearate is added as a lubricant. The mixture is formed into 450-900 mg tablets (150-300 mg of active amide compound) in a tablet press.

### Formulation 5—Injection

A compound of the invention may be dissolved or suspended in a buffered sterile saline injectable aqueous medium to a concentration of approximately 5 mg/ml.

### Formulation 6—Topical

Stearyl alcohol (250 g) and a white petrolatum (250 g) may be melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) is added and the resulting mixture is stirred until it congeals.

### Methods of Treatment

The present compounds are used as therapeutic agents for the treatment of conditions in mammals that are causally related or attributable to aberrant activity of the Wnt/wg signaling pathway. Accordingly, the compounds and pharmaceutical compositions of this invention find use as therapeutics for preventing and/or treating a variety of cancers and hyperproliferative conditions in mammals, including humans. Thus, and as stated earlier, the present invention includes within its scope, and extends to, the recited methods of treatment, as well as to the compounds for use in such methods, and for the preparation of medicaments useful for such methods.

In a method of treatment aspect, this invention provides a method of treating a mammal susceptible to or afflicted with a condition associated with cancer and/or a hyperproliferative disorder, which method comprises administering an effective amount of one or more of the pharmaceutical compositions just described.

In yet another method of treatment aspect, this invention provides a method of treating a mammal susceptible to or afflicted with a condition that gives rise to increased cellular proliferation or a transformed phenotype, or that relates to dysregulation of Wnt/wg signaling. The present oxazoles have use as anti-proliferative agents that reduce proliferative levels (potentially to normal levels for a particular cell type), and/or anti-transformed phenotype agents that restore, at least in part, normal phenotypic properties of a particular cell type. Accordingly, the present oxazoles have use for the treatment of cancers and hyperproliferative disorders and fibrotic diseases relating to aberrant Wnt/wg signaling.

In additional method of treatment aspects, this invention provides methods of treating a mammal susceptible to or afflicted with a cancer causally related or attributable to aberrant activity of the Wnt/wg signaling pathway. Such cancers include, without limitation, those of the prostate cancer, colon cancer, rectal cancer, breast cancer, skin cancer (e.g., melanoma), liver cancer (e.g., hepatocellular cancer and hepatoblastoma), head and neck cancer, lung cancer (e.g., non-small cell lung cancer), gastric cancer, mesothelioma, Barrett's esophagus, synovial sarcoma, cervical cancer, endometrial ovarian cancer, Wilm's tumor, bladder cancer and leukemia. Such methods comprise administering an effective condition-treating or condition-preventing amount of one or more of the pharmaceutical compositions just described.

Also envisioned herein are combination therapies that comprise administration of one or more of the oxazole compounds described herein, or a composition thereof, in conjunction with therapeutic regimens (e.g., local and/or systemic therapeutic modalities) implemented for treating patients afflicted with diseases or conditions that are causally related to the aberrant activity of the Wnt pathway in vivo. Such diseases include, without limitation, pulmonary fibrosis; and cancers, including: without limitation, prostate cancer, colon cancer, rectal cancer, breast cancer, skin cancer (e.g., melanoma), liver cancer (e.g., hepatocellular cancer and hepatoblastoma), head and neck cancer, lung cancer (e.g., non-small cell lung cancer), gastric cancer, mesothelioma, Barrett's esophagus, synovial sarcoma, cervical cancer, endometrial ovarian cancer, Wilm's tumor, bladder cancer and leukemia.

Such combination therapies can boost the therapeutic activity of each of the therapeutic modalities with the potential for synergistic therapeutic benefit. Combination therapy, furthermore, has the potential to improve therapeutic benefit with no significant increase in morbidity relative to that typically achieved using the individual therapeutic modalities (e.g., monotherapies) separately. Under some circumstances, the doses of each of the individual therapeutic modalities can be reduced, which may result in an overall decrease in morbidity when combination therapy is implemented.

It will be appreciated that combination therapies may involve administration of one or more of the oxazole compounds described herein, or a composition thereof, at the same time, before, and/or after a second therapeutic modality of the combination therapy. The timing of administration of the, e.g., first and second therapeutic modalities of a combination therapy may be determined based on the experience of the attending physician and the manner in which therapeutic modalities known in the art are typically administered.

In a particular aspect, a combination therapy comprises administration of one or more of the oxazole compounds described herein, or a composition thereof, in conjunction with a systemic therapeutic modality, such as, for example, one or more of a systemic inhibitor of immune system down regulation, such as anti-CTLA-4 (including but not limited to ipilimumab and tremelimumab), PD-1, and PD-L1 antibodies.

In one aspect, combination therapy may comprise administration of one or more of the oxazole compounds described herein, or a composition thereof, in conjunction with one or more of a systemic immune upregulating agent, including: non-specific cytokines, such as interleukin-1, -2, or -6 (IL-1, IL-2 or IL-6) and aldesleukin; interferon-alpha or gamma (IFN-α and IFN-γ), interferon alfa-2b and pegylated interferon (including pegylated interferon alfa-2a and pegylated interferon alfa-2b); granulocyte macrophage colony stimulating factor (GM-CSF, molgramostim or sargramostim); dendritic cell vaccines and other allogeneic or autologous therapeutic cancer vaccines, including intralesional vaccines containing an oncolytic herpes virus encoding GM-CSF (ONCOVEX®) or a plasmid encoding human leukocyte antigen-B7 and beta-2 microglobulin agent designed to express allogeneic MHC class I antigens (ALLOVECTIN-7®); and antibodies against specific tumor antigens.

In yet another aspect, combination therapy may comprise administration of one or more of the oxazole compounds described herein, or a composition thereof, in conjunction with one or more systemic targeted therapy agent, including: drugs that target protein kinases and the receptors that activate them, including but not limited to afatinib (BIBW 2992), bevacizumab, cetuximab, dasatinib, E7080, erlotinib, gefitinib, imatinib, lapatinib, nilotinib, panitumumab, pazopanib, pegaptanib, ranibizumab, sorafenib, sunitinib, trastuzumab and vandetanib; serine/threonine-selective protein kinase inhibitors, including but not limited to those targeting the B-Raf/MEKIERK pathway, such as vemurafenib (also known as PLX4032, RG7204 or RO5185426), GSK2118436 and GSK1120212; aromatase inhibitors, including but not limited to aminoglutethimide, anastrozole, exemestane, fadrozole, formestane, letrozole, testolactone and vorozole; estrogen receptor antagonists, including but not limited to lasofoxifene, raloxifene, tamoxifen and toremifene; COX-2 inhibitors, including but not limited to celecoxib, valdecoxib and rofecoxib; angiogenesis blockers, including IFN-α, IL-12, suramin, and thrombospondin (including thrombospondin 1, ABT-510 and ABT-898); and immune cell therapy, including but not limited to adoptive T-cell transfer and autologous immune cell therapy.

The aforementioned systemic therapeutic modalities are known in the art and are described in, for example, U.S. 2015/0290318, the entire content of which is incorporated herein by reference.

In a further aspect, a combination therapy comprises administration of one or more of the oxazole compounds described herein, or a composition thereof, in conjunction with a local therapeutic modality, such as, for example, a local immunomodulative therapy: including, but not limited to, intralesional (IL) chemoablation using an IL chemoablative agent consisting primarily of rose bengal (4,5,6,7-tetrachloro-2′,4′,5′,7′-tetraiodofluorescein) or another halogenated xanthene, including erythrosin B, phloxine B, 4,5,6,7-tetrabromo-2′,4′,5′,7′-tetraiodofluorescein, 2′,4,5,6,7-pentachloro-4′,5′,7′-triiodofluorescein, 4,4′,5,6,7-pentachloro-2′,5′,7′-triiodofluorescein, 2′,4,5,6,7,7′-hexachloro-4′,5′-diiodofluorescein, 4,4′,5,5′,6,7-hexachloro-2′,7′-diiodofluorescein, 2′,4,5,5′,6,7-hexachloro-4′,7′-diiodofluorescein, 4,5,6,7-tetrachloro-2′,4′,5′-triiodofluorescein, 4,5,6,7-tetrachloro-2′,4′,7′-triiodofluorescein, 4,5,6,7-tetrabromo-2′,4′,5′-triiodofluorescein, and 4,5,6,7-tetrabromo-2′,4′,7′-triiodofluorescein in an appropriate pharmaceutical composition, including a 0.1% (w/v) or higher concentration aqueous solution of rose bengal (i.e., PV-10) or equivalent solution of another halogenated xanthene or mixtures thereof. A physiologically acceptable salt of the halogenated xanthene may be used in this composition. The aforementioned local immunomodulative therapies are known in the art and are described in, for example, U.S. 2015/0290318 and U.S. application Ser. No. 12/315,781, the entire content of each of which is incorporated herein by reference.

In a more particular aspect, a combination therapy comprises administration of one or more of the oxazole compounds described herein, or a composition thereof, in conjunction with intralesional (IL) chemoablation with PV-10 or another halogenated xanthene agent. As described in, for example, U.S. 2015/0290318 (the entire content of which is incorporated herein by reference), IL chemoablation using a specific class of agent (for example certain formulations of certain halogenated xanthenes, as exemplified by a 10% (w/v) solution of rose bengal disodium in saline, termed “PV-10”) can elicit not only highly specific ablation of the injected lesion but also an antitumor immune response (“bystander effect”) that augments local efficacy in the injected tumor and leads to spontaneous regression of uninjected tumors. PV-10 is, for example, undergoing clinical testing for treatment of metastatic melanoma, breast carcinoma and hepatocellular carcinoma. IL chemoablation can lead to rapid reduction in tumor burden, thereby reducing the potential for tumor-induced immune suppression and potentially minimizing the extent and severity of the disease. Chemoablation of entire tumors or substantially all of the tumors, and especially chemoablation of multiple tumors, enhances exposure of the patient's immune system to distinct clonal subpopulations of tumor cells that may be present, and thus maximizes overall immune response to the tumor.

The effects of combination therapy can be heightened by repeated administration. Since IL chemoablation is well suited to repeat treatment, continued therapeutic intervention by ongoing administration of the oxazole compounds described herein, or a composition thereof, in conjunction with repeated IL chemoablation is envisioned. The timing of administration may be varied and combination therapy may be performed with concurrent administration of either therapy, or delayed administration of one or another of the therapies.

Under circumstances wherein disease is rapidly proliferating, widely disseminated, or presents in a form difficult to infiltrate fully with the IL chemoablative agent, use of additional complementary therapeutic modalities may offer additive or synergistic benefit, particularly when they promote immunologic stimulation (i. e., immunomodulation). Complementary immunomodulative therapies may be used to advantage to promote additive or synergistic immunologic interactions that allow one or multiple therapies to be used at reduced doses relative to those used when administered individually as monotherapies, while retaining high efficacy and potentially reducing undesirable adverse effects. Exemplary immunomodulative therapies elicit immune system upregulation or counter tumor-induced immune system down regulation.

Monotherapy dose schedules are set by determining the maximum tolerated dose (MTD) in early-stage clinical trials. The MTD (or a close variation thereof) is then applied to later-stage clinical trials that optimize efficacy and further assess issues pertaining to safety. Exemplary dosing schedules for a number of systemic agents that may be combined with administration of one or more of the oxazole compounds described herein, or a composition thereof, are provided in Table 1.

TABLE 1
Example systemic immunomodulatory or targeted anticancer agents

| Systemic Agent | Typical Dose Schedule | |
|---|---|---|
| Ipilimumab | 3 | mg/kg q21d for 4 treatments |
| Tremelimumab | 15 | mg/kg q3m |
| Aldesleukin | 600,000 | IU/kg q8h (up to 14 doses before 9 |
| | day rest and repeat; rest at least 7 wks | |
| | before repeat of course) | |
| interferon alfa-2b | 20 | million IU/m² 5 times per week |
| | for 4 weeks (induction phase) followed | |
| | by 10 million IU/m² three times per week | |
| | (maintenance phase) | |
| pegylated interferon | 6 | μg/kg qwk for eight weeks |
| | (induction phase) followed by 3 μg/kg | |
| | qwk (maintenance phase) | |
| Oncovex ® | 4 | mL IL at 10⁸ pfu/mL |
| GM-CSF | 125 | μg/m² daily for 14 wks followed |
| | by 14 days rest | |
| Allovectin-7 ® | 2 | mg IL qwk for 6 wks |
| Afatinib | 20-50 | mg daily |
| Bevacizumab | 5-15 | mg/kg q14d-q21d |
| Cetuximab | 400 | mg/m² followed by weekly |
| | maintenance at 250 mg/m² | |
| Dasatinib | 100 | mg daily |
| Erlotinib | 100-150 | mg daily |
| Gefitinib | 250 | mg daily |
| Imatinib | 400-600 | mg daily (increased to twice daily |
| | if well tolerated or disease progresses) | |
| Lapatinib | 1250 | mg daily for 21 day cycle |
| Nilotinib | 400 | mg twice daily |
| Panitumumab | 6 | mg/kg ql4d |
| Pazopanib | 800 | mg daily |
| Pegaptanib | 0.3 | mg q6wks |
| Ranibizumab | 0.5 | mg q4wks |
| Sorafenib | 400 | mg twice daily |
| Sunitinib | 50 | mg daily for 4 weeks followed |
| | by 2 week recovery | |
| Trastuzumab | 4 | mg/kg followed by weekly |
| | maintenance at 2 mg/kg | |
| Vandetanib | 200-300 | mg daily |
| Vemurafenib | 960 | mg twice daily |
| (PLX4032) | | |
| GSK2118436 ^{a} | 150 | mg twice daily |
| GSK1120212 ^{a} | 2 | mg daily |
| aminoglutethimide | 250 | mg q6h |
| Anastrozole | 1 | mg daily |
| Exemestane | 25 | mg daily |
| Fadrozole | 1 | mg twice daily |
| Foimestane | 250 | mg daily |
| Letrozole | 2.5 | mg daily |
| Vorozole | 2.5 | mg daily |
| Raloxifene | 60 | mg daily |
| Tamoxifen | 20-40 | mg daily |
| Toremifene | 60 | mg daily |
| Celecoxib | 200-400 | mg twice daily |
| Rofecoxib | 20-25 | mg daily |
| Suramin | 1 | g qwk |
| thrombospondin | 20 | mg daily to 100 mg twice daily |

(ABT-510 ^{a})

^{a} Proprietary code name for drug under development for which no nonproprietary name is
currently available.

Due to additive or synergistic effects of the monotherapies used in combination, the combination therapies and methods for treatment described herein will generally permit administration of a systemic agent at a level at or below the typical dose schedule for the systemic agent when used as a monotherapy (as described in Table 1). This may also apply to dosing parameters for the oxazole compounds described herein or a composition thereof. Lower doses of the oxazole compounds described herein, or a composition thereof, may confer benefit when used in combination with either systemic or a local therapeutic modality, such as, for example, a local immunomodulative therapy.

Further to the above, combination therapies that comprise administration of one or more of the oxazole compounds described herein, or a composition thereof, in conjunction with therapeutic regimens directed to promoting immune responses are envisioned. Such therapeutic regimens may be directed to promoting immune responses systemically and/or in a localized manner. As indicated herein above, effective systemic immunotherapeutic approaches have been developed and implemented for the treatment of a variety of cancers, including melanoma, lung cancer, and prostate cancer. These approaches include blockade of immune-inhibitory receptors on activated T cells. Monoclonal antibodies against CTLA-4, PD-1, and PD-L1, for example, have been used to advantage to promote immune responses. See, for example, Kaufman et al. (2013, Nature Rev Clin Oncol 10:588); Mellman et al. (2011, Nature 480:480); Wolchok et al. (2013, N Engl J Med 369:122); Topalian et al. (2014, J Cin Oncol 32:1020); and Hodi et al. (2010, N Engl J Med 363:711), the entire content of each of which is incorporated herein by reference. Combination therapy comprising inhibitors of Wnt/β-catenin pathways and antibodies against CTLA-4, PD-1, and/or PD-L1 have been proposed as having potential for the treatment of, for example, melanoma. Without being bound by theory, it is thought that inhibitors of Wnt/β-catenin pathways enhance the efficacy of the immunotherapy. See, for example, Spranger et al. (2015, Nature 523:231); Hanks et al. (2015, J Clin Oncol 33:suppl. abstr 3054); Sweis et al. (2015, J Clin Oncol 33:suppl. abstr 4511); Spranger et al. (2015, J Clin Oncol 33:suppl. abstr 9014), the entire content of which each of which is incorporated herein by reference. Accordingly, combination therapy comprising administration of one or more of the oxazole compounds described herein, or a composition thereof, in conjunction with at least one of antibodies against CTLA-4, PD-1, and/or PD-L1 is envisioned herein.

Various types of cancers are listed herein and are envisioned for treatment using the combination therapies described herein. Such combination therapies comprise administration of one or more of the oxazole compounds described herein, or a composition thereof, in conjunction with a therapeutic regimen used for the treatment of a cancer, such as those understood in the art and listed, for example, herein below.

Breast Cancer

Cancer drugs approved by the Food and Drug Administration (FDA) for treating breast cancer include, without limitation: Abitrexate (Methotrexate), Abraxane (Paclitaxel Albumin-stabilized Nanoparticle Formulation) Ado-Trastuzumab Emtansine, Adrucil (Fluorouracil), Afinitor (Everolimus), Anastrozole, Aredia (Pamidronate Disodium), Arimidex (Anastrozole), Aromasin (Exemestane), Capecitabine, Clafen (Cyclophosphamide), Cyclophosphamide, Cytoxan (Cyclophosphamide), Docetaxel, Doxorubicin Hydrochloride, Efudex (Fluorouracil), Ellence (Epirubicin Hydrochloride), Epirubicin Hydrochloride, Eribulin Mesylate, Everolimus, Exemestane, 5-FU (Fluorouracil), Fareston (Toremifene), Faslodex (Fulvestrant), Femara (Letrozole), Fluoroplex (Fluorouracil), Fluorouracil, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Ixabepilone, Ixempra (Ixabepilone), Kadcyla (Ado-Trastuzumab Emtansine), Lapatinib Ditosylate, Letrozole, Megace (Megestrol Acetate), Megestrol Acetate, Methotrexate, Methotrexate LPF (Methotrexate), Mexate (Methotrexate), Mexate-AQ (Methotrexate), Neosar (Cyclophosphamide), Nolvadex (Tamoxifen Citrate), Paclitaxel, Paclitaxel Albumin-stabilized Nanoparticle Formulation, Palbociclib, Pamidronate Disodium, Perjeta (Pertuzumab), Pertuzumab, Tamoxifen Citrate, Taxol (Paclitaxel), Taxotere (Docetaxel), Thiotepa, Toremifene, Trastuzumab, Tykerb (Lapatinib Ditosylate), Velban (Vinblastine Sulfate), Velsar (Vinblastine Sulfate), Vinblastine Sulfate, Xeloda (Capecitabine), and Zoladex (Goserelin Acetate).

Drug combinations used for treating breast include, without limitation: AC: Doxorubicin Hydrochloride (Adriamycin) (A); and Cyclophosphamide (C); AC-T: Doxorubicin Hydrochloride (Adriamycin) (A); Cyclophosphamide (C); Paclitaxel (Taxol) (T); CAF: Cyclophosphamide (C); Doxorubicin Hydrochloride (Adriamycin) (A); and Fluorouracil (F); CMF: Cyclophosphamide (C); Methotrexate (M); and Fluorouracil (F); FEC: Fluorouracil (F); Epirubicin Hydrochloride (E); Cyclophosphamide (C); TAC: Docetaxel (Taxotere) (T); Doxorubicin Hydrochloride (Adriamycin) (A); and Cyclophosphamide (C).

Additional information pertaining to monotherapies and drug combinations used for treating breast cancer are known in the art and can be accessed via a variety websites on the worldwide web, including those provided by the National Cancer Institute.

Prostate Cancer

Cancer drugs approved by the FDA for treating prostate cancer include, without limitation: Abiraterone Acetate; Bicalutamide; Cabazitaxel; Casodex (Bicalutamide); Degarelix; Docetaxel; Enzalutamide; Goserelin Acetate; Jevtana (Cabazitaxel); Leuprolide Acetate; Lupron (Leuprolide Acetate); Lupron Depot (Leuprolide Acetate); Lupron Depot-3 Month (Leuprolide Acetate); Lupron Depot-4 Month (Leuprolide Acetate); Lupron Depot-Ped (Leuprolide Acetate); Mitoxantrone Hydrochloride; Prednisone; Provenge (Sipuleucel-T); Radium 223 Dichloride; Sipuleucel-T; Taxotere (Docetaxel); Viadur (Leuprolide Acetate); Xofigo (Radium 223 Dichloride); Xtandi (Enzalutamide); Zoladex (Goserelin Acetate); and Zytiga (Abiraterone Acetate).

Additional information pertaining to monotherapies and drug combinations used for treating prostate cancer are known in the art and can be accessed via a variety websites on the worldwide web, including those provided by the National Cancer Institute.

Colon Cancer

Cancer drugs approved by the FDA for treating colon cancer include, without limitation: Adrucil (Fluorouracil); Avastin (Bevacizumab); Bevacizumab; Camptosar (Irinotecan Hydrochloride); Capecitabine; Cetuximab; Cyramza (Ramucirumab); Efudex (Fluorouracil); Eloxatin (Oxaliplatin); Erbitux (Cetuximab); 5-FU (Fluorouracil); Fluoroplex (Fluorouracil); Fluorouracil; Irinotecan Hydrochloride; Leucovorin Calcium; Lonsurf (Trifluridine and Tipiracil Hydrochloride); Oxaliplatin; Panitumumab; Ramucirumab; Regorafenib; Stivarga (Regorafenib); Trifluridine and Tipiracil Hydrochloride; Vectibix (Panitumumab); Wellcovorin (Leucovorin Calcium); Xeloda (Capecitabine); Zaltrap (Ziv-Aflibercept); and Ziv-Aflibercept.

Drug combinations used for treating colon cancer include, without limitation: CAPOX: Capecitabine (CAP); Oxaliplatin (OX); FOLFIRI: Leucovorin Calcium (Folinic Acid) (FOL); Fluorouracil (F); Irinotecan Hydrochloride (TM); FOLFIRI-BEVACIZUMAB: Leucovorin Calcium (Folinic Acid) (FOL); Fluorouracil (F); Irinotecan Hydrochloride (TM); Bevacizumab; FOLFIRI-CETUXIMAB: Leucovorin Calcium (Folinic Acid) (FOL); Fluorouracil (F); Irinotecan Hydrochloride (IRI); Cetuximab; FOLFOX: Leucovorin Calcium (Folinic Acid) (FOL); Fluorouracil (F); Oxaliplatin (OX); FU-LV: Fluorouracil (FU); Leucovorin Calcium (LV); and XELIRI: Capecitabine (Xeloda) (XEL); Irinotecan Hydrochloride (IRI); XELOX: Capecitabine (Xeloda) (XEL); Oxaliplatin (OX).

Rectal Cancer

Cancer drugs approved by the FDA for treating rectal cancer include, without limitation: Adrucil (Fluorouracil); Avastin (Bevacizumab); Bevacizumab; Camptosar (Irinotecan Hydrochloride); Capecitabine; Cetuximab; Cyramza (Ramucirumab); Efudex (Fluorouracil); Eloxatin (Oxaliplatin); Erbitux (Cetuximab); 5-FU (Fluorouracil); Fluoroplex (Fluorouracil); Fluorouracil; Irinotecan Hydrochloride; Leucovorin Calcium; Lonsurf (Trifluridine and Tipiracil Hydrochloride); Oxaliplatin; Panitumumab; Ramucirumab; Regorafenib; Stivarga (Regorafenib); Trifluridine and Tipiracil Hydrochloride; Vectibix (Panitumumab); Wellcovorin (Leucovorin Calcium); Xeloda (Capecitabine); Zaltrap (Ziv-Aflibercept); and Ziv-Aflibercept.

Drug combinations used for treating rectal cancer include, without limitation: CAPOX: Capecitabine (CAP); Oxaliplatin (OX); FOLFIRI: Leucovorin Calcium (Folinic Acid) (FOL); Fluorouracil (F); Irinotecan Hydrochloride (IRI); FOLFIRI-BEVACIZUMAB: Leucovorin Calcium (Folinic Acid) (FOL); Fluorouracil (F); Irinotecan Hydrochloride (IRI); Bevacizumab; FOLFIRI-CETUXIMAB: Leucovorin Calcium (Folinic Acid) (FOL); Fluorouracil (F); Irinotecan Hydrochloride (IRI); Cetuximab; FOLFOX: Leucovorin Calcium (Folinic Acid) (FOL); Fluorouracil (F); Oxaliplatin (OX); FU-LV: Fluorouracil (FU); Leucovorin Calcium (LV); XELIRI: Capecitabine (Xeloda) (XEL); Irinotecan Hydrochloride (IRI); XELOX: Capecitabine (Xeloda) (XEL); and Oxaliplatin (OX).

Gastroenteropancreatic Neuroendocrine Tumors

Cancer drugs approved by the FDA for treating gastroenteropancreatic neuroendocrine tumors_include, without limitation: Lanreotide Acetate; and Somatuline Depot (Lanreotide Acetate).

Additional information pertaining to monotherapies and drug combinations used for treating colon cancer, rectal cancer, and gastrenteropancreatic neuroendocrine tumors are known in the art and can be accessed via a variety websites on the worldwide web, including those provided by the National Cancer Institute.

Ovarian Cancer, Fallopian Tube Cancer, and Primary Peritoneal Cancer

Cancer drugs approved by the FDA for treating ovarian cancer, fallopian tube cancer, and primary peritoneal cancer include, without limitation: Avastin (Bevacizumab); Bevacizumab; Carboplatin; Clafen; (Cyclophosphamide); Cisplatin; Cyclophosphamide; Cytoxan (Cyclophosphamide); Doxorubicin Hydrochloride; Dox-SL (Doxorubicin Hydrochloride Liposome); DOXIL (Doxorubicin Hydrochloride Liposome); Doxorubicin Hydrochloride Liposome; Evacet (Doxorubicin Hydrochloride Liposome); Gemcitabine Hydrochloride; Gemzar (Gemcitabine Hydrochloride); Hycamtin (Topotecan Hydrochloride); LipoDox (Doxorubicin Hydrochloride Liposome); Lynparza (Olaparib); Neosar (Cyclophosphamide); Olaparib; Paclitaxel; Paraplat (Carboplatin); Paraplatin (Carboplatin); Platinol (Cisplatin); Platinol-AQ (Cisplatin); Taxol (Paclitaxel); Thiotepa; and Topotecan Hydrochloride.

Drug combinations used for treating ovarian cancer, fallopian tube cancer, and primary peritoneal cancer include, without limitation: BEP: Bleomycin (B); Etoposide (E); Cisplatin (Platinol) (P); CARBOPLATIN-TAXOL: Carboplatin (C); Paclitaxel (Taxol); GEMCITABINE-CISPLATIN: Gemcitabine Hydrochloride, Cisplatin; and VeIP: Vinblastine Sulfate (Velban) (Ve); Ifosfamide (I); Cisplatin (Platinol).

Liver Cancer

Cancer drugs approved by the FDA for treating liver cancer include, without limitation: Nexavar (Sorafenib Tosylate); and Sorafenib Tosylate.

Head and Neck Cancer

Cancer drugs approved by the FDA for treating cancer that arises in the head or neck region (in the nasal cavity, sinuses, lips, mouth, salivary glands, throat, or larynx [voice box]) include, without limitation: Abitrexate (Methotrexate); Adrucil (Fluorouracil); Blenoxane (Bleomycin); Bleomycin; Cetuximab; Cisplatin; Docetaxel; Efudex (Fluorouracil); Erbitux (Cetuximab); 5-FU (Fluorouracil); Fluoroplex (Fluorouracil); Fluorouracil; Folex (Methotrexate); Folex PFS (Methotrexate); Methotrexate; Methotrexate LPF (Methotrexate); Mexate (Methotrexate); Mexate-AQ (Methotrexate); Platinol (Cisplatin); Platinol-AQ (Cisplatin); and Taxotere (Docetaxel).

Drug combinations used for treating head and neck cancer include, without limitation: TPF: Docetaxel (Taxotere) (T); Cisplatin (Platinol) (P); and Fluorouracil (F).

Leukemia

Cancer drugs approved by the FDA for treating acute lymphoblastic leukemia (ALL) include, without limitation: Abitrexate (Methotrexate); Arranon (Nelarabine); Asparaginase Erwinia chrysanthemi; Blinatumomab; Blincyto (Blinatumomab); Cerubidine (Daunorubicin Hydrochloride); Clafen (Cyclophosphamide); Clofarabine; Clofarex (Clofarabine); Clolar (Clofarabine); Cyclophosphamide; Cytarabine; Cytosar-U (Cytarabine); Cytoxan (Cyclophosphamide); Dasatinib; Daunorubicin Hydrochloride; Doxorubicin Hydrochloride; Erwinaze (Asparaginase Erwinia Chrysanthemi); Folex (Methotrexate); Folex PFS (Methotrexate); Gleevec (Imatinib Mesylate); Iclusig (Ponatinib Hydrochloride); Imatinib Mesylate; Marqibo (Vincristine Sulfate Liposome); Mercaptopurine; Methotrexate; Methotrexate LPF (Methorexate); Mexate (Methotrexate); Mexate-AQ (Methotrexate); Nelarabine; Neosar (Cyclophosphamide); Oncaspar (Pegaspargase); Pegaspargase; Ponatinib Hydrochloride; Prednisone; Purinethol (Mercaptopurine); Purixan (Mercaptopurine); Rubidomycin (Daunorubicin Hydrochloride); Sprycel (Dasatinib); Tarabine PFS (Cytarabine); Vincasar PFS (Vincristine Sulfate); Vincristine Sulfate; and Vincristine Sulfate Liposome.

Drug combinations used for treating ALL include, without limitation: Hyper-CVAD: Cyclophosphamide (C); Vincristine Sulfate (V); and Doxorubicin Hydrochloride (Adriamycin).

Cancer drugs approved by the FDA for treating acute myeloid leukemia (AML) include, without limitation: Arsenic Trioxide; Cerubidine (Daunorubicin Hydrochloride); Clafen (Cyclophosphamide); Cyclophosphamide; Cytarabine; Cytosar-U (Cytarabine); Cytoxan (Cyclophosphamide); Daunorubicin Hydrochloride; Doxorubicin Hydrochloride; Idamycin (Idarubicin Hydrochloride); Idarubicin Hydrochloride; Mitoxantrone Hydrochloride; Neosar (Cyclophosphamide); Rubidomycin (Daunorubicin Hydrochloride); Tabloid (Thioguanine); Tarabine PFS (Cytarabine); Thioguanine; Trisenox (Arsenic Trioxide); Vincasar PFS (Vincristine Sulfate); and Vincristine Sulfate.

Drug combinations used for treating AML include, without limitation: ADE: Cytarabine (Ara-C); Daunorubicin Hydrochloride (D); and Etoposide (E).

Cancer drugs approved by the FDA for treating chronic lymphocytic leukemia (CLL) include, without limitation: Alemtuzumab; Ambochlorin (Chlorambucil); Amboclorin (Chlorambucil); Arzerra (Ofatumumab); Bendamustine Hydrochloride; Campath (Alemtuzumab); Chlorambucil; Clafen (Cyclophosphamide); Cyclophosphamide; Cytoxan (Cyclophosphamide); Fludara (Fludarabine Phosphate); Fludarabine Phosphate; Gazyva (Obinutuzumab); Ibrutinib; Idelalisib; Imbruvica (Ibrutinib); Leukeran (Chlorambucil); Linfolizin (Chlorambucil); Mechlorethamine Hydrochloride; Mustargen (Mechlorethamine Hydrochloride); Neosar (Cyclophosphamide); Obinutuzumab; Ofatumumab; Prednisone; Rituxan (Rituximab); Rituximab; Treanda (Bendamustine Hydrochloride); and Zydelig (Idelalisib).

Drug combinations used for treating CLL include, without limitation: CHLORAMBUCIL-PREDNISONE; and CVP: Cyclophosphamide (C); Vincristine Sulfate (V); and Prednisone (P).

Cancer drugs approved by the FDA for treating chronic myelogenous leukemia (CIVIL) include, without limitation: Bosulif (Bosutinib); Bosutinib; Busulfan; Busulfex (Busulfan); Clafen (Cyclophosphamide); Cyclophosphamide; Cytarabine; Cytosar-U (Cytarabine); Cytoxan (Cyclophosphamide); Dasatinib; Gleevec (Imatinib Mesylate); Iclusig (Ponatinib Hydrochloride); Imatinib Mesylate; Mechlorethamine Hydrochloride; Mustargen (Mechlorethamine Hydrochloride); Myleran (Busulfan); Neosar (Cyclophosphamide); Nilotinib; Omacetaxine Mepesuccinate; Ponatinib Hydrochloride; Sprycel (Dasatinib); Synribo (Omacetaxine Mepesuccinate); Tarabine PFS (Cytarabine); and Tasigna (Nilotinib).

Cancer drugs approved by the FDA for treating hairy cell leukemia include, without limitation: Intron A (Recombinant Interferon Alfa-2b); and Recombinant Interferon Alfa-2b.

Cancer drugs approved by the FDA for treating meningeal leukemia include, without limitation: Cytarabine; Cytosar-U (Cytarabine); and Tarabine PFS (Cytarabine).

Lung Cancer

Cancer drugs approved by the FDA for treating non-small cell lung cancer include, without limitation: Abitrexate (Methotrexate); Abraxane (Paclitaxel Albumin-stabilized Nanoparticle Formulation); Afatinib Dimaleate; Alimta (Pemetrexed Disodium); Avastin (Bevacizumab); Bevacizumab; Carboplatin; Ceritinib; Cisplatin; Crizotinib; Cyramza (Ramucirumab); Docetaxel; Erlotinib Hydrochloride; Folex (Methotrexate); Folex PFS (Methotrexate); Gefitinib; Gilotrif (Afatinib Dimaleate); Gemcitabine Hydrochloride; Gemzar (Gemcitabine Hydrochloride); Iressa (Gefitinib); Keytruda (Pembrolizumab); Mechlorethamine Hydrochloride; Methotrexate; Methotrexate LPF (Methotrexate); Mexate (Methotrexate); Mexate-AQ (Methotrexate); Mustargen (Mechlorethamine Hydrochloride); Navelbine (Vinorelbine Tartrate); Nivolumab; Opdivo (Nivolumab); Paclitaxel; Paclitaxel Albumin-stabilized Nanoparticle Formulation; Paraplat (Carboplatin); Paraplatin (Carboplatin); Pembrolizumab; Pemetrexed Disodium; Platinol (Cisplatin); Platinol-AQ (Cisplatin); Ramucirumab; Tarceva (Erlotinib Hydrochloride); Taxol (Paclitaxel); Taxotere (Docetaxel); Vinorelbine Tartrate; Xalkori (Crizotinib); and Zykadia (Ceritinib).

Drug combinations used for treating non-small cell lung cancer include, without limitation: Carboplatin-Taxol and Gemcitabine-Cisplatin.

Cancer drugs approved by the FDA for treating small cell lung cancer include, without limitation: Abitrexate (Methotrexate); Doxorubicin Hydrochloride; Etopophos (Etoposide Phosphate); Etoposide; Etoposide Phosphate; Folex (Methotrexate); Folex PFS (Methotrexate); Hycamtin (Topotecan Hydrochloride); Mechlorethamine Hydrochloride; Methotrexate; Methotrexate LPF (Methotrexate); Mexate (Methotrexate); Mexate-AQ (Methotrexate); Mustargen (Mechlorethamine Hydrochloride); Toposar (Etoposide); Topotecan Hydrochloride; and VePesid (Etoposide).

Skin Cancer

Cancer drugs approved by the FDA for treating basal cell carcinoma include, without limitation: Adrucil (Fluorouracil); Aldara (Imiquimod); Efudex (Fluorouracil); Erivedge (Vismodegib); 5-FU (Fluorouracil); Fluoroplex (Fluorouracil); Fluorouracil; Imiquimod; Odomzo (Sonidegib); Sonidegib; and Vismodegib.

Cancer drugs approved by the FDA for treating melanoma include, without limitation: Aldesleukin; Dabrafenib; Dacarbazine; DTIC-Dome (Dacarbazine); IL-2 (Aldesleukin); Imlygic (Talimogene Laherparepvec); Interleukin-2 (Aldesleukin); Intron A (Recombinant Interferon Alfa-2b); Ipilimumab; Keytruda (Pembrolizumab); Mekinist (Trametinib); Nivolumab; Opdivo (Nivolumab); Peginterferon Alfa-2b; Pembrolizumab; Proleukin (Aldesleukin); Recombinant Interferon Alfa-2; Sylatron (Peginterferon Alfa-2b); Tafinlar (Dabrafenib); Talimogene Laherparepvec; Trametinib; Vemurafenib; Yervoy (Ipilimumab); and Zelboraf (Vemurafenib).

Gastric (Stomach) Cancer

Cancer drugs approved by the FDA for treating melanoma include, without limitation: Adrucil (Fluorouracil); Cyramza (Ramucirumab); Docetaxel; Doxorubicin Hydrochloride; Efudex (Fluorouracil); 5-FU (Fluorouracil); Fluoroplex (Fluorouracil); Fluorouracil; Herceptin (Trastuzumab); Mitomycin C; Mitozytrex (Mitomycin C); Mutamycin (Mitomycin C); Ramucirumab; Taxotere (Docetaxel); and Trastuzumab.

Drug combinations used for treating gastric cancer include, without limitation: FU-LV; TPF; and XELIRI.

Malignant Mesothelioma

Cancer drugs approved by the FDA for treating malignant mesothelioma include, without limitation: Alimta (Pemetrexed Disodium); Cisplatin; Pemetrexed Disodium; Platinol (Cisplatin); and Platinol-AQ (Cisplatin).

Drug combinations used for treating malignant mesothelioma include, without limitation: GEMCITABINE-CISPLATIN.

Esophageal Cancer

Cancer drugs approved by the FDA for treating esophageal cancer include, without limitation: Cyramza (Ramucirumab); Docetaxel; Herceptin (Trastuzumab); Ramucirumab; Taxotere (Docetaxel); and Trastuzumab.

Drug combinations used for treating malignant esophageal cancer include, without limitation: FU-LV and XELIRI.

Synovial Sarcoma

Drug combinations used for treating synovial sarcoma include, without limitation: Doxorubicin and/or Ifosfamide. The primary treatment for synovial sarcoma is surgical resection, preferably with clear margins.

Cervical Cancer

Cancer drugs approved by the FDA for treating cervical cancer include, without limitation: Avastin (Bevacizumab); Bevacizumab; Blenoxane (Bleomycin); Bleomycin; Cisplatin; Hycamtin (Topotecan Hydrochloride); Platinol (Cisplatin); Platinol-AQ (Cisplatin); and Topotecan Hydrochloride.

Drug combinations used for treating cervical cancer include, without limitation: Gemcitabine-Cisplatin.

Bladder Cancer

Cancer drugs approved by the FDA for treating bladder cancer include, without limitation: Cisplatin; Doxorubicin Hydrochloride; Platinol (Cisplatin); Platinol-AQ (Cisplatin); and Thiotepa.

Drug combinations used for treating bladder cancer include, without limitation: Gemcitabine-Cisplatin.

Wilms Tumor

Drugs used for treating Wilms Tumor include, without limitation: actinomycin D, vincristine, doxorubicin, cyclophosphamide, etoposide, carboplatin, mesna (to protect the bladder from the effects of the cyclophosphamide), and irinotecan, and combinations thereof.

It is, moreover, understood that the oxazole compounds described herein, or a composition thereof, may be administered in conjunction with a therapeutic regimen that calls for radiation and/or surgery. The order in which combination therapies are administered may vary depending on, for example, the cancer to be treated, the stage of the cancer, and the general health of the patient afflicted with the cancer. The oxazole compounds described herein, or a composition thereof, may, for example, be administered alone or in a combination therapy to shrink a tumor in advance of surgical resection and/or may be administered after surgical resection to minimize or ablate residual disease.

In further method of treatment aspects, methods are presented for treating a mammal susceptible to or afflicted with pulmonary fibrosis, wherein such methods comprise administering an effective condition-treating or condition-preventing amount of one or more of the pharmaceutical compositions described herein. Idiopathic pulmonary fibrosis (IPF) is a progressive disease with a high mortality rate. Dysregulation of the Wnt/β-catenin pathway has been implicated in lung fibrosis. See, for example, Lam et al, (2014, Am J Resp Crit Care Med 190:185); Selman et al. (2008, PLoS Med 5:e62); Henderson et al. (2010, Proc Natl Acad Sci USA 107: 14309); Ulsamer et al. (2012, J Biol Chem 287:5164); Akhmetshina et al. (2012, Nat Commun 3:735); and Konigshoff et al. (2009, J Clin Invest 119:772); the entire content of each of which is incorporated herein by reference.

Also envisioned herein are combination therapies for the treatment of IPF. Such combination therapies comprise administration of at least one of the present oxazoles in combination with a therapeutic regimen currently applied to the care of patients suffering from pulmonary fibrosis. Guidelines for the care of IPF are known in the art and provided by the American Thoracic Society, European Respiratory Society, Japanese Respiratory Society, and the Latin American Thoracic Association. The salient points regarding guidelines for therapeutic intervention are as follows: use of nintedanib, a tyrosine kinase inhibitor that targets multiple tyrosine kinases, including vascular endothelial growth factor, fibroblast growth factor, and PDGF receptors; pirfenidone; and antacid therapy, even in patients without symptoms of gastroesophageal reflux (GER), are conditionally recommended. Pirfenidone (Esbriet), available from Roche Holding AG, and nintedanib (Ofev), available from Boehringer Ingelheim GmbH, received FDA approval for treating IPF in 2014. The guidelines also provide a conditional recommendation against using N-acetylcysteine monotherapy, sildenafil, macitentan, and bosentan. The guidelines further recommend against the use of anticoagulation agents (warfarin); imatinib, a selective tyrosine kinase inhibitor against platelet-derived growth factor (PDGF) receptors; combination prednisone, azathioprine, and N-acetylcysteine; and selective endothelin receptor antagonist (ambrisentan).

As is understood in the art, people who are diagnosed with pulmonary fibrosis may initially be treated with a corticosteroid (e.g., prednisone), sometimes in combination with other drugs that suppress the immune system (e.g., methotrexate or cyclosporine). Adding acetylcysteine, a derivative of a natural amino acid, to prednisone may slow the disease in some people. Accordingly, the combination therapy encompassed herein may comprise administration of at least one of the present oxazoles in combination with a corticosteroid alone or in combination with an immunosuppressant such as methotrexate or cyclosporine. Combination therapy encompassed herein may also comprise administration of at least one of the present oxazoles in combination with oxygen therapy, pulmonary rehabilitation, and/or surgery. All of the above therapeutic interventions are known in the art to be implemented for treating pulmonary fibrosis and are described in various websites, including that provided by the Mayo Clinic.

As a further aspect of the invention there is provided the present compounds for use as a pharmaceutical especially in the treatment or prevention of the aforementioned conditions and diseases. Also provided herein is the use of the present compounds in the manufacture of a medicament for the treatment or prevention of one of the aforementioned conditions and diseases.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, such as psoriasis, the regimen for treatment usually stretches over many months or years so oral dosing is preferred for patient convenience and tolerance. Psoriasis, for example, has been linked to Wnt signaling. Several basic and clinical studies using patient samples revealed an increase in nuclear β-catenin staining in many psoriatic samples. It has been suggested that a sustained low-level increase in Wnt/β-catenin signaling could be responsible for skin psoriatic lesions. With oral dosing, one to five and especially two to four and typically three oral doses per day are representative regimens. Using these dosing patterns, each dose provides from about 0.01 to about 20 mg/kg of the compound of the invention, with preferred doses each providing from about 0.1 to about 10 mg/kg and especially about 1 to about 5 mg/kg.

Transdermal doses are generally selected to provide similar or lower blood levels than are achieved using injection doses.

When used to prevent the onset of a hyperproliferative condition, the compounds of this invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The compounds of this invention can be administered as the sole active agent or they can be administered in combination with other agents, including other compounds that demonstrate the same or a similar therapeutic activity, and that are determined to safe and efficacious for such combined administration.

### General Synthetic Procedures

The compounds of this invention may be purchased from various commercial sources or can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, N.Y., 1991, and references cited therein.

The following schemes are presented with details as to the preparation of representative compounds that have been listed hereinabove. The compounds of the invention may be prepared from known or commercially available starting materials and reagents by one skilled in the art of organic synthesis.

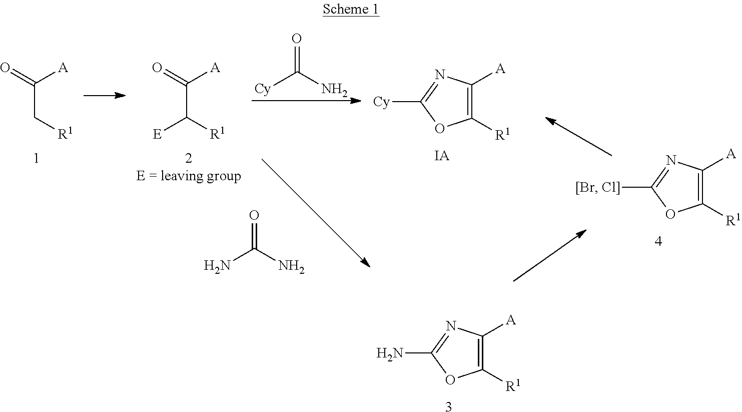

Compounds of the invention may be prepared according to Scheme 1. Ketones 1 containing the appropriate A and R¹ substituents may be transformed into 2 containing a suitable leaving group E, such as a halide or sulfonate, in the a-position, which may be further transformed directly into compounds of formula IA by treatment with an appropriately functionalized amide. Alternatively, 2 may be reacted with a urea to give the amino-substituted heterocycles 3. Intermediates 3 may be transformed into the corresponding halides 4, which when coupled with an appropriate reagent, such as an aryl or heteroaryl stannane or boron-containing group, may provide compounds of formula IA.

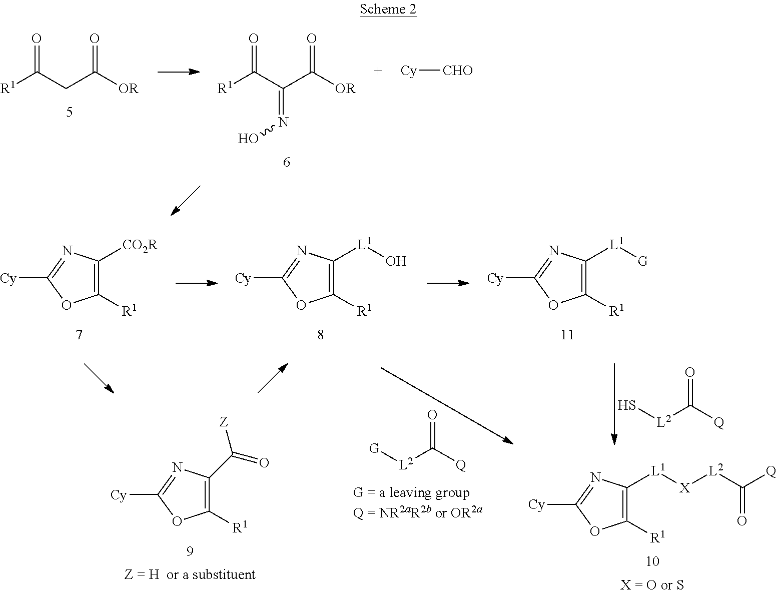

In an alternative approach (Scheme 2), β-ketoesters 5 may be reacted with a nitrite salt to provide compounds of structure 6, which when reacted with an appropriate aryl or heteroaryl aldehyde may provide oxazoles 7 containing an ester substituent. Compounds 7 may also be prepared by methods similar to those described in Scheme 1, and may be transformed by methods known to those skilled in the art to alcohols 8. Alternatively, esters 7 may be transformed into the appropriate carbonyl-containing intermediates 9, which may be further reacted to provide alcohols 8. Compounds of the invention 10 where X═O may be prepared directly from 8 by reaction with an appropriately substituted alkylating intermediate in the presence of a strong base such as sodium hydride. For compounds 10 where X═S, alcohols 8 may be transformed into the alkylating intermediates 11, which when treated with the appropriate thiol, may provide the corresponding compounds of the invention. Alternatively, alcohols 8 may be transformed into the corresponding thiol and reacted with an alkylating agent to provide 10 (X═S); or, the appropriate alcohol may be reacted with intermediates 11 to provide 10 (X═O).

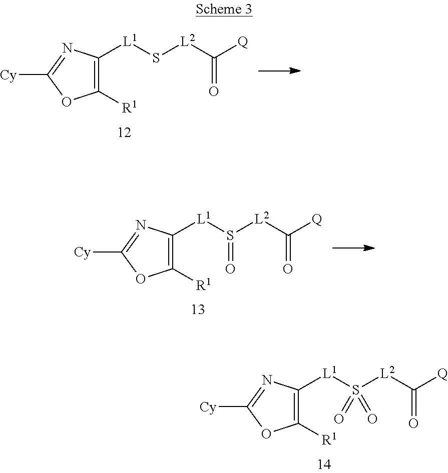

Compounds of the invention where X═SO, SO₂ may be readily prepared as shown in Scheme 3. Sulfide-containing intermediates 12 may be partially oxidized to the sulfoxides 13 using one equivalent of an oxidizing agent, such as meta-chloroperoxybenzoic acid, or transformed directly into the sulfones 14 through the use of an excess of an oxidizing reagent. Isolated sulfoxides 13 may also be transformed into the corresponding sulfones 14.

### EXAMPLE 1

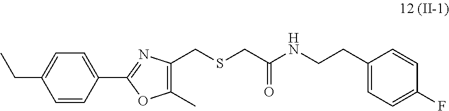 [SMILES: CCc1ccc(-c2nc(CSCC(=O)NCCc3ccc(F)cc3)c(C)o2)cc1]

### Step-1

### Preparation of 2-[(E)-hydroxyimino]-3-oxo-butyric acid ethyl ester (2)

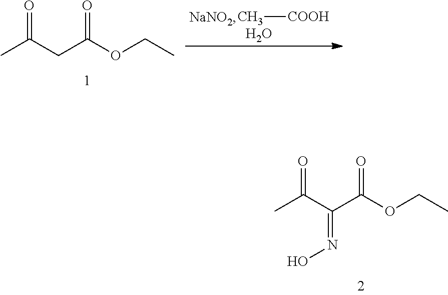

To a solution of ethyl acetoacetate (1) (2 g, 0.115 mol, 1 eq) in acetic acid (6 mL) at 0° C., a solution of sodium nitrite (1.06 g, 0.115 mol, 1 eq) in water (4 mL) was added dropwise and stirred for 30 min at 25° C. The resulting reaction mixture was diluted with ethyl acetate (100 mL), and the organic layer was washed with water (2×50 mL), dried over sodium sulfate and concentrated under reduced pressure to obtain the product 2 as a pale yellow liquid (2.3 g, 98%)

### Step-2

### Preparation of 2-(4-ethyl-phenyl)-5-methyl-3-oxy-oxazole-4-carboxylic acid ethyl ester (4)

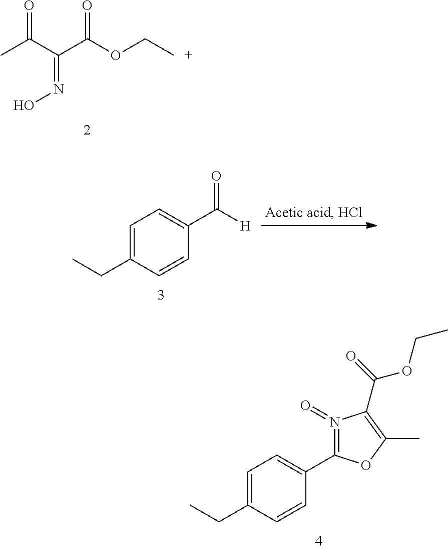

A solution of 2-[(E)-hydroxyimino]-3-oxo-butyric acid ethyl ester (2) (1 g, 0.006 moles, 1 eq) in acetic acid (7 mL), 4-ethyl-benzaldehyde (3) (1.12 g, 0.0093 moles, 1.5 eq) was added at 25° C. and then cooled to 0° C. To the resulting solution, dry HCl gas was bubbled for 30 min at the same temperature (until the solution became wine red in color). The reaction mixture was gradually warmed to 25° C. and stirred for 3 h at the same temperature. The resulting reaction mixture was added slowly to vigorously stirred diethyl ether (100 mL) to obtain product 4 as a white solid, which was filtered through a Buchner funnel, dried under vacuum and taken to the next step without further purification (800 mg, 48%).

### Step-3

### Preparation of 2-(4-ethyl-phenyl)-5-methyl-oxazole-4-carboxylic acid ethyl ester (5)

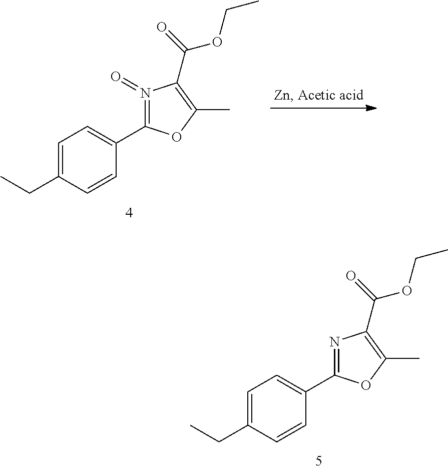 [SMILES: CCOC(=O)c1c(C)oc(-c2ccc(CC)cc2)[n+]1[O-] ; CCOC(=O)c1nc(-c2ccc(CC)cc2)oc1C]

To a solution of 2-(4-ethyl-phenyl)-5-methyl-3-oxy-oxazole-4-carboxylic acid ethyl ester (4) (600 mg, 0.002 moles, 1 eq) in acetic acid (6 mL) at 0° C., Zn dust (450 mg, 0.0068 moles, 3 eq) was added portionwise, and the resulting suspension was allowed to stir at 25° C. for 20 min. Then the reaction mixture was quenched with aqueous HCl (1.5N) and filtered through a bed of Celite. The filtrate obtained was suspended in a mixture of water (200 mL) and ethyl acetate (200 mL). The ethyl acetate layer was separated, dried over anhydrous sodium sulfate and concentrated under reduced pressure to obtain product 5 as a white solid (400 mg, 70%).

### Step-4

### Preparation of [2-(4-ethyl-phenyl)-5-methyl-oxazol-4-yl]-methanol (6)

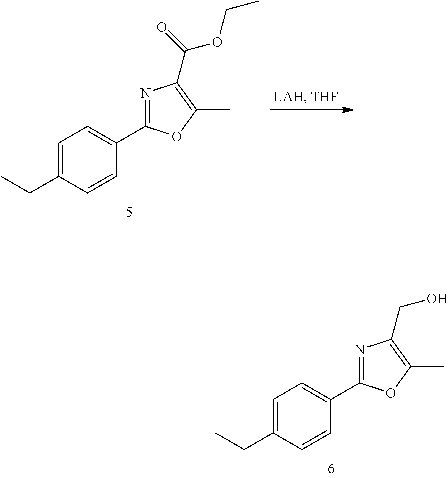 [SMILES: CCOC(=O)c1nc(-c2ccc(CC)cc2)oc1C ; CCc1ccc(-c2nc(CO)c(C)o2)cc1]

To a suspension of lithium aluminum hydride (100 mg, 0.003 moles, 1.5 eq) in THF (10 mL) at −20° C., a solution of 2-(4-ethyl-phenyl)-5-methyl-oxazole-4-carboxylic acid ethyl ester (5) (500 mg, 0.0019 moles, 1 eq) in THF (3 mL) was added dropwise, warmed and allowed to stir at 25° C. for 30 min. The resulting reaction mixture was quenched with aqueous ammonium chloride (10%) at 0° C. and diluted with ethyl acetate (150 mL). The suspension formed was filtered through a bed of Celite, and the ethyl acetate layer was separated, dried over anhydrous sodium sulfate and concentrated under reduced pressure to obtain product 6 as a white solid (250 mg, 62%).

### Step-5

### Preparation of 4-chloromethyl-2-(4-ethyl-phenyl)-5-methyl-oxazole (7)

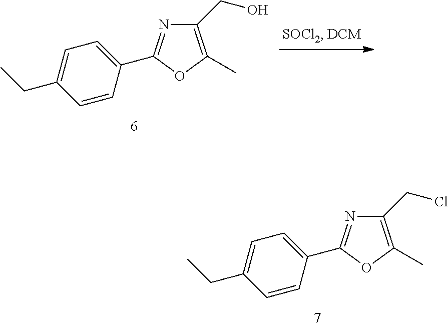 [SMILES: CCc1ccc(-c2nc(CO)c(C)o2)cc1 ; CCc1ccc(-c2nc(CCl)c(C)o2)cc1]

To a solution of [2-(4-ethyl-phenyl)-5-methyl-oxazol-4-yl]-methanol (6) (3.7 g, 0.017 moles, 1 eq) in DCM (37 mL) at 0° C., thionyl chloride (1.16 mL, 0.022 moles, 1.3 eq) was added dropwise and stirred at 25° C. for 30 min. The resulting reaction mixture was diluted with ethyl acetate (500 mL), washed with water (250 mL) and aqueous sodium bicarbonate (10%) (250 mL), dried over anhydrous sodium sulfate and concentrated under reduced pressure to obtain product 7 as an off-white solid (3.6 g, 90%).

### Step-6

### Preparation of [2-(4-ethyl-phenyl)-5-methyl-oxazol-4-ylmethylsulfanyl]-acetic acid ethyl ester (9)

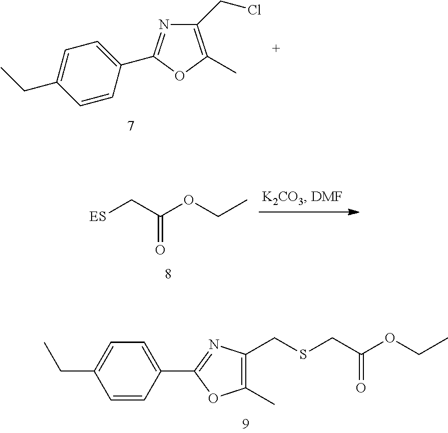

To a solution of 4-chloromethyl-2-(4-ethyl-phenyl)-5-methyl-oxazole (7) (9.1 g, 0.38 mol, 1 eq) in DMF (45 mL) at 25-30° C. were added potassium carbonate (13.3 g, 0.96 moles, 2.2 eq), DMAP (catalytic) and ethyl thioacetate (8) (5.9 mL, 0.54 mol, 1.4 eq). The reaction mixture was allowed to stir for 45 min at the same temperature. The resulting reaction mass was diluted with water (500 mL) and extracted with ethyl acetate (2×300 mL). The combined organic layers were dried over anhydrous sodium sulfate and concentrated under reduced pressure to obtain product 9 as an off-white solid (9.5 g 76%).

### Step-7

### Preparation of [2-(4-ethyl-phenyl)-5-methyl-oxazol-4-ylmethylsulfanyl]-acetic acid (10)

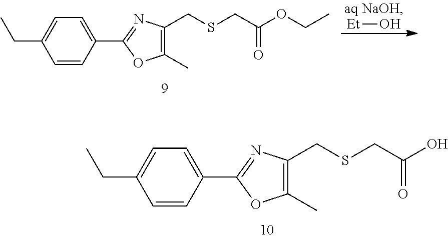

To a solution of [2-(4-ethyl-phenyl)-5-methyl-oxazol-4-ylmethylsulfanyl]-acetic acid ethyl ester (9) (9.5 g, 0.029 mol, 1 eq) in methanol at 25° C. was added an aqueous solution of sodium hydroxide (3.58 g, 0.089 mol) (19 mL) and the reaction mixture was stirred for 30 min at the same temperature. The resulting reaction mixture was concentrated under reduced pressure to remove methanol, and the residue obtained was dissolved in water and acidified to pH 1 using aqueous HCl (1.5N) to obtain a white precipitate, which was filtered using a Buchner funnel. The solid obtained was dissolved in DCM (100 mL), dried over anhydrous sodium sulfate and concentrated under reduced pressure to obtain product 10 as a white solid (6.5 g, 75%).

MS (ESI) m/z: 292.1 (M+H)⁺

¹H NMR (400 MHz, CDCl₃) δ 7.89 (d, J=8.3 Hz, 2H), 7.29 (d, J=8.3 Hz, 2H), 3.80 (s, 2H), 3.38 (s, 2H), 2.70 (q, J=7.6 Hz, 2H), 2.40 (s, 3H), 1.26 (t, J=7.6 Hz, 3H).

### Step-8

### Preparation of 2-[2-(4-ethyl-phenyl)-5-methyl-oxazol-4-ylmethylsulfanyl]-N-[2-(4-fluoro-phenyl)-ethyl]-acetamide (12/II-1)

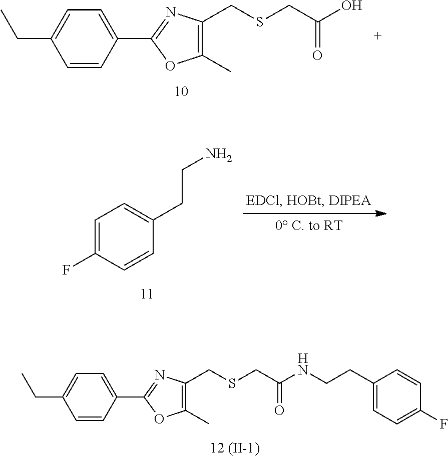 [SMILES: CCc1ccc(-c2nc(CSCC(=O)O)c(C)o2)cc1 ; "+" ; NCCc1ccc(F)cc1 ; CCc1ccc(-c2nc(CSCC(=O)NCCc3ccc(F)cc3)c(C)o2)cc1]

To a solution of [2-(4-ethyl-phenyl)-5-methyl-oxazol-4-yl methylsulfanyl]-acetic acid (10) (0.5 g, 1.17 mmol, 1 eq) in DMF (5 mL), DIPEA (0.525 mL, 2.925 mmol, 2.5 eq), EDCI (350 mg, 1.79 mmol, 1.5 eq) and HOBt (33 mg, 0.234 mol, 0.2 eq) were added at 0° C. and stirred for 5 min at the same temperature. To the resulting reaction mixture was added 4-fluoro-phenethylamine (11) (178 mg, 1.287 mmol, 1.1 eq) at 0° C. and the reaction mixture was stirred for 30 min at 25° C. The reaction mixture was diluted with water (50 mL) and extracted with ethyl acetate. The ethyl acetate layer was washed with aqueous sodium bicarbonate solution (10%, 50 mL), aqueous HCl (1.5N, 50 mL), dried over anhydrous sodium sulfate and concentrated under reduced pressure to obtain the crude product as an off-white solid, which was further purified by column chromatography using silica gel (230-400) in a hexane/ethyl acetate solvent system to obtain pure product 12 as a white solid (350 mg, 48%).

MS (ESI) m/z: 413.6 (M+H)⁺

¹H NMR (300 MHz, DMSO-d₆) δ 8.03 (br s, 1H), 7.81 (d, J=8.1 Hz, 2H), 7.33 (d, J=8.1 Hz, 2H), 7.20 (m, 2H), 7.07 (t, J=9.0 Hz, 2H), 3.68 (s, 2H), 3.22-3.34 (m, 2H), 3.09 (s, 2H), 2.63-2.70 (m, 4H), 2.34 (s, 3H), 1.19 (t, J=7.5 Hz, 3H).

### EXAMPLE 2

The following analogs were synthesized from compound 10 by following the synthetic protocol described in step 8.

| | MS (ESI) m/z (M + H)⁺ |
|---|---|
| 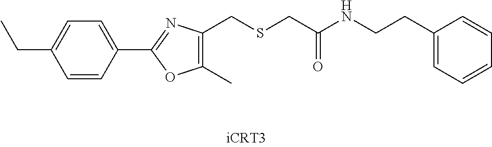 [SMILES: CCc1ccc(-c2nc(CSCC(=O)NCCc3ccccc3)c(C)o2)cc1] | 395.1 |
| 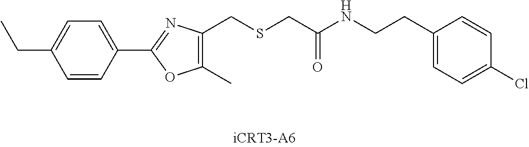 [SMILES: CCc1ccc(-c2nc(CSCC(=O)NCCc3ccc(Cl)cc3)c(C)o2)cc1] | 429.0, 431.0 |
| 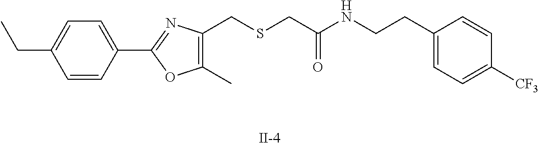 [SMILES: CCc1ccc(-c2nc(CSCC(=O)NCCc3ccc(C(F)(F)F)cc3)c(C)o2)cc1 ; "II-4"] | 463.1 |
| 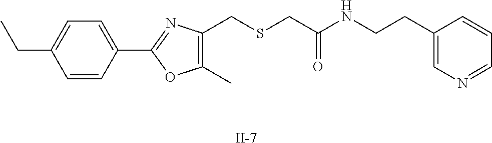 [SMILES: CCc1ccc(-c2nc(CSCC(=O)NCCc3cccnc3)c(C)o2)cc1 ; "II-7"] | 396.5 |
| 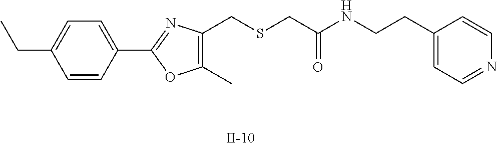 [SMILES: CCc1ccc(-c2nc(CSCC(=O)NCCc3ccncc3)c(C)o2)cc1 ; "II-10"] | 396.2 |
| 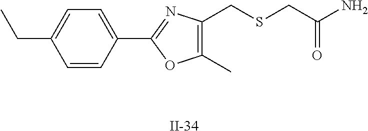 [SMILES: CCc1ccc(-c2nc(CSCC(N)=O)c(C)o2)cc1 ; "II-34"] | 291.1 |

### EXAMPLE 3

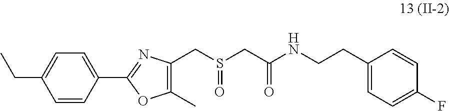 [SMILES: CCc1ccc(-c2nc(CS(=O)CC(=O)NCCc3ccc(F)cc3)c(C)o2)cc1]

### Preparation of 2-[2-(4-ethyl-phenyl)-5-methyl-oxazol-4-ylmethanesulfinyl]-N-[2-(4-fluoro-phenyl)-ethyl]-acetamide (13/II-2)

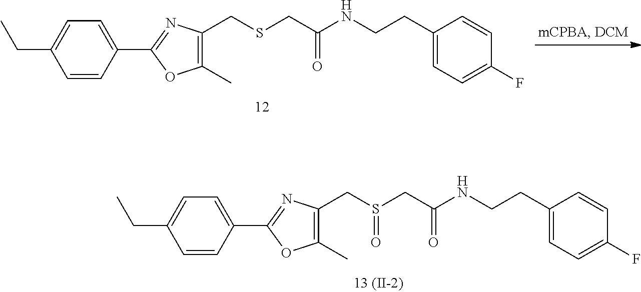 [SMILES: CCc1ccc(-c2nc(CSCC(=O)NCCc3ccc(F)cc3)c(C)o2)cc1 ; CCc1ccc(-c2nc(CS(=O)CC(=O)NCCc3ccc(F)cc3)c(C)o2)cc1]

To a solution of 2-[2-(4-ethyl-phenyl)-5-methyl-oxazol-4-ylmethylsulfanyl]-N-[2-(4-fluoro-phenyl)-ethyl]-acetamide (12) (100 mg, 0.23 mmol, 1 eq) in DCM (5 mL), 3-chloroperbenzoic acid (70%) (57 mg, 0.23 mmol, 1 eq) was added at 0° C., warmed and stirred for 30 min at 25° C. The resulting reaction mixture was diluted with DCM (100 mL), washed with aqueous sodium bicarbonate solution (10%, 50 mL) and water (75 mL), and dried over sodium sulfate. The crude product obtained upon evaporation of the solvent was purified by silica gel (230-400) column chromatography (50% hexane in ethyl acetate) to obtain pure product 13 as a white solid (60 mg, 58%).

MS (ESI) m/z: 429.6 (M+H)⁺

¹H NMR (300 MHz, DMSO-d₆) δ 8.39 (br s, 1H), 7.83 (d, J=8.1 Hz, 2H), 7.35 (d, J=8.1 Hz, 2H), 7.24 (m, 2H), 7.08 (t, J=9.0 Hz, 2H), 4.16 (d, J=13 Hz, 1H), 4.02 (d, J=13 Hz, 1H), 3.80 (d, J=13 Hz, 1H), 3.59 (d, J=13 Hz, 1H), 3.25-3.40 (m, 2H), 2.62-2.72 (m, 4H), 2.37 (s, 3H), 1.19 (t, J=7.5 Hz, 3H).

### EXAMPLE 4

The sulfoxide analogs below were synthesized from the corresponding sulfide analogs by following the oxidation protocol described in step 9.

| | MS (ESI) m/z (M + H)⁺ |
|---|---|
| 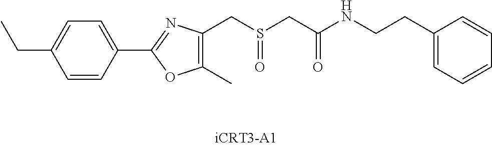 [SMILES: CCc1ccc(-c2nc(CS(=O)CC(=O)NCCc3ccccc3)c(C)o2)cc1] | 411.5 |
| 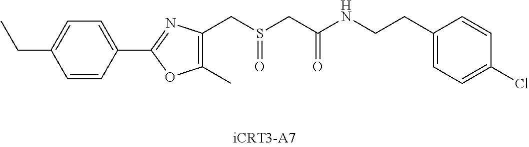 [SMILES: CCc1ccc(-c2nc(CS(=O)CC(=O)NCCc3ccc(Cl)cc3)c(C)o2)cc1] | 445.1 |
| 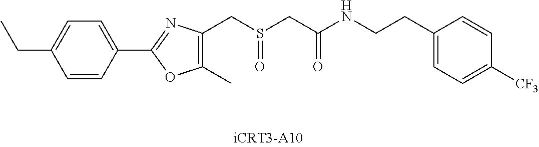 [SMILES: CCc1ccc(-c2nc(CS(=O)CC(=O)NCCc3ccc(C(F)(F)F)cc3)c(C)o2)cc1] | 479.1 |
| 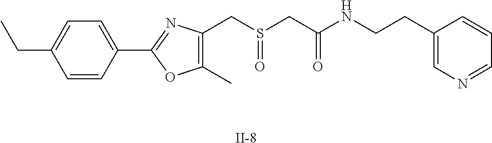 [SMILES: CCc1ccc(-c2nc(CS(=O)CC(=O)NCCc3cccnc3)c(C)o2)cc1 ; "II-8"] | 412.0 |
| 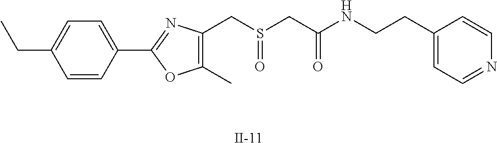 [SMILES: CCc1ccc(-c2nc(CS(=O)CC(=O)NCCc3ccncc3)c(C)o2)cc1 ; "II-11"] | 412.2 |

### EXAMPLE 5

### Preparation of (II-3) 2-[2-(4-ethyl-phenyl)-5-methyl-oxazol-4-ylmethanesulfonyl]-N-[2-(4-fluoro-phenyl)-ethyl]-acetamide (14)

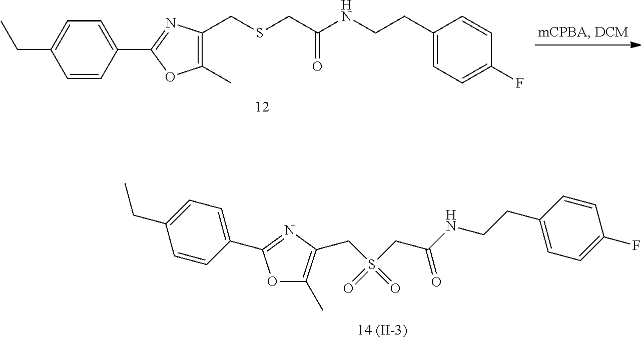 [SMILES: CCc1ccc(-c2nc(CSCC(=O)NCCc3ccc(F)cc3)c(C)o2)cc1 ; CCc1ccc(-c2nc(CS(=O)(=O)CC(=O)NCCc3ccc(F)cc3)c(C)o2)cc1]

To a solution of 2-[2-(4-ethyl-phenyl)-5-methyl-oxazol-4-ylmethylsulfanyl]-N-[2-(4-fluoro-phenyl)-ethyl]-acetamide (12) (100 mg, 0.23 mmol, 1 eq) in DCM (5 mL), 3-chloroperbenzoic acid (70%) (125 mg, 0.506 mmol, 2.2 eq) was added at 0° C., warmed and stirred for 30 min at 25° C. The resulting reaction mixture was diluted with dichloromethane (100 mL), washed with aqueous sodium bicarbonate solution (10%, 50 mL) and water (75 mL), and dried over sodium sulfate. The crude product obtained upon evaporation of the solvent was purified by silica gel (230-400) column chromatography (30% hexane in ethyl acetate) to obtain pure product 14 as a white solid (65 mg, 60%).

MS (ESI) m/z: 445.1 (M+H)⁺

¹H NMR (300 MHz, DMSO-d₆) δ 8.45 (br s, 1H), 7.85 (d, J=8.1 Hz, 2H), 7.37 (d, J=8.1 Hz, 2H), 7.27 (dd, J=5.7, 20 Hz, 2H), 7.11 (t, J=9.0 Hz, 2H), 4.64 (s, 2H), 4.13 (s, 2H), 3.25-3.40 (m, 2H), 2.75 (t, J=7.1 Hz, 2H), 2.66 (t, J=7.7 Hz, 2H), 2.42 (s, 3H), 1.21 (t, J=7.5 Hz, 3H).

### EXAMPLE 6

The sulfone analogs below were synthesized from the corresponding sulfide analogs by following the oxidation protocol described in step 10.

| | MS (ESI) m/z (M + H)⁺ |
|---|---|
| 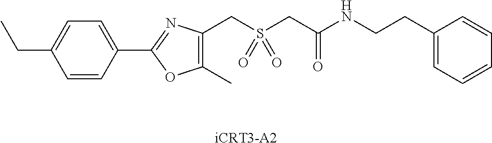 [SMILES: CCc1ccc(-c2nc(CS(=O)(=O)CC(=O)NCCc3ccccc3)c(C)o2)cc1 ; "iCRT3-A2"] | 427.5 |
| 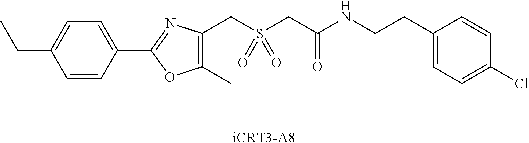 [SMILES: CCc1ccc(-c2nc(CS(=O)(=O)CC(=O)NCCc3ccc(Cl)cc3)c(C)o2)cc1 ; "iCRT3-A8"] | 461.8 |
| 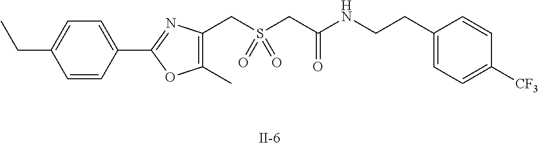 [SMILES: CCc1ccc(-c2nc(CS(=O)(=O)CC(=O)NCCc3ccc(C(F)(F)F)cc3)c(C)o2)cc1 ; "II-6"] | 495.1 |
| 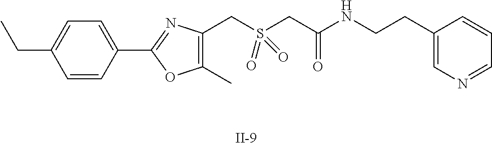 [SMILES: CCc1ccc(-c2nc(CS(=O)(=O)CC(=O)NCCc3cccnc3)c(C)o2)cc1 ; "II-9"] | 428.3 |
| 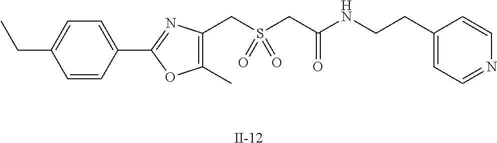 [SMILES: CCc1ccc(-c2nc(CS(=O)(=O)CC(=O)NCCc3ccncc3)c(C)o2)cc1 ; "II-12"] | 428.2 |

### EXAMPLE 7

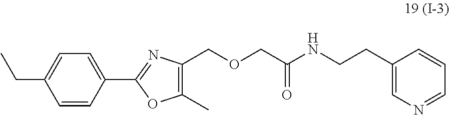 [SMILES: CCc1ccc(-c2nc(COCC(=O)NCCc3cccnc3)c(C)o2)cc1]

### Step-1

### Preparation of [2-(4-ethyl-phenyl)-5-methyl-oxazol-4-ylmethoxy]-acetic acid ethyl ester (16)

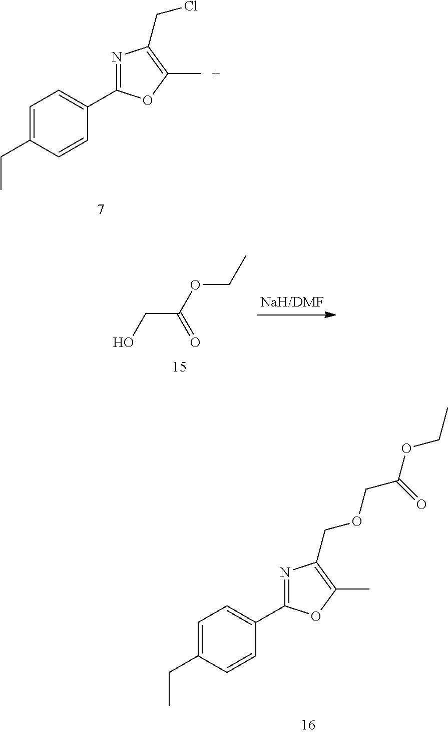

To a solution of 4-chloromethyl-2-(4-ethyl-phenyl)-5-methyl-oxazole (7) (0.5 g, 2.12 mmol) in DMF (5 mL), NaH (60% in mineral oil) (169 mg, 4.24 mmol, 2 eq) was added at 0° C., and the reaction mixture was stirred for 10 min at the same temperature. To the reaction mixture was added ethyl glycotate (15) (325 mg, 3.18 mmol, 1.5 eq) at 0° C., and the reaction mixture was allowed to warm and stirred at 25° C. for 4 h. The reaction mixture was quenched with ice-cold aqueous 1.5N HCl at 0° C., diluted with ethyl acetate (100 mL), and washed with water (75 mL). The organic phase was dried over sodium sulfate and concentrated under reduced pressure to obtain the crude compound as a yellow liquid. The crude product obtained upon evaporation of the solvent was purified by silica gel (230-400) column chromatography (30% hexane in ethyl acetate) to obtain pure product 16 as a white solid (300 mg, 46%).

### Step-2

### Preparation of [2-(4-ethyl-phenyl)-5-methyl-oxazol-4-ylmethoxy]-acetic acid (17)

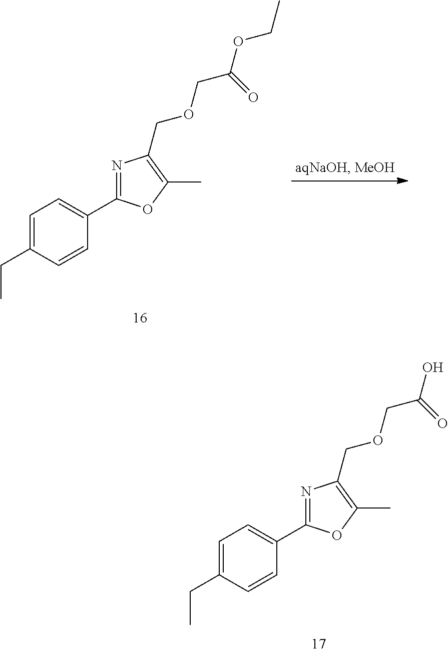 [SMILES: CCOC(=O)COCc1nc(-c2ccc(CC)cc2)oc1C ; CCc1ccc(-c2nc(COCC(=O)O)c(C)o2)cc1]

To a solution of [2-(4-ethyl-phenyl)-5-methyl-oxazol-4-ylmethoxy]-acetic acid ethyl ester (16) (300 mg, 0.99 mmol, 1 eq) in methanol (6 mL) at 25° C. was added an aqueous solution of sodium hydroxide (118 mg, 2.97 mmol, 3 eq) (2 mL) and the mixture was stirred for 30 min at the same temperature. The resulting reaction mixture was concentrated under reduced pressure to remove methanol, and the residue obtained was dissolved in water and acidified to pH 1 with aqueous HCl (1.5N) to obtain a white precipitate, which was filtered using a Buchner funnel. The solid obtained was dissolved in DCM (100 mL), dried over anhydrous sodium sulfate and concentrated under reduced pressure to obtain product 17 as a white solid (230 mg, 83%).

### Step-3

### Preparation of (II-53), 2-[2-(4-Ethyl-phenyl)-5-methyl-oxazol-4-ylmethoxy]-N-(2-pyridin-3-yl-ethyl)-acetamide (19)

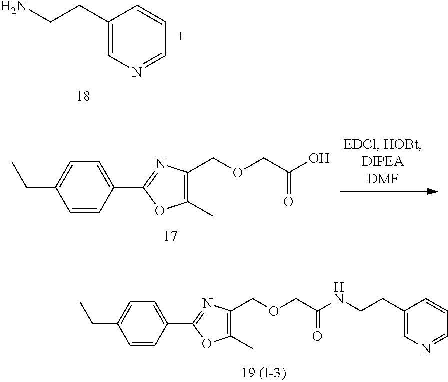

To a solution of [2-(4-ethyl-phenyl)-5-methyl-oxazol-4-ylmethoxy]-acetic acid (17) (100 mg, 0.363 mmol, 1 eq) in DMF (2.5 mL), DIPEA (0.16 mL, 0.909 mmol, 2.5 eq), EDCI (110 mg, 0.545 mmol, 1.5 eq) and HOBT (10 mg, 0.072 mmol, 0.2 eq) were added at 0° C. and stirred for 5 min at the same temperature. To the resulting reaction mixture at 0° C., 2-pyridin-3-yl-ethylamine (50 mg, 0.399 mmol, 1.1 eq) was added and stirred for 2 hat 25° C. The resulting reaction mixture was diluted with water (50 mL) and extracted with ethyl acetate (2×75 mL). The combined ethyl acetate layers were washed with aqueous sodium bicarbonate solution (10%, 50 mL), dried over anhydrous sodium sulfate and concentrated under reduced pressure. The crude product obtained upon evaporation of the solvent was purified by silica gel (230-400) column chromatography (5% methanol in chloroform) to obtain pure product 19 as a white solid (50 mg, 37%).

MS (ESI) m/z: 380.2 (M+H)⁺

¹H NMR (300 MHz, DMSO-d₆) δ 8.39 (br s, 2H), 7.80-7.92 (m, 3H), 7.60 (d, J=8.1 Hz, 1H), 7.35 (d, J=8.1 Hz, 2H), 7.25-7.31 (m, 1H), 4.40 (s, 2H), 3.88 (s, 2H), 2.60-2.79 (m, 4H), 2.37 (s, 3H), 1.20 (t, J=7.5 Hz, 3H).

### EXAMPLE 8

The oxygen analogs below were synthesized by following the experimental procedure described in Example 7.

| | MS (ESI) m/z (M + H)⁺ |
|---|---|
| 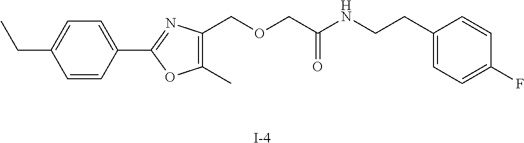 | 397.6 |
| 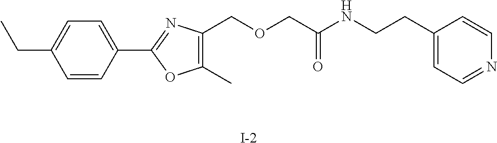 | 380.2 |

### EXAMPLE 9

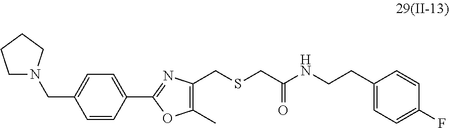 [SMILES: Cc1oc(-c2ccc(CN3CCCC3)cc2)nc1CSCC(=O)NCCc1ccc(F)cc1]

### Step-1

### Preparation of 2-(4-methoxymethyl-phenyl)-5-methyl-3-oxy-oxazole-4-carboxylic acid ethyl ester (21)

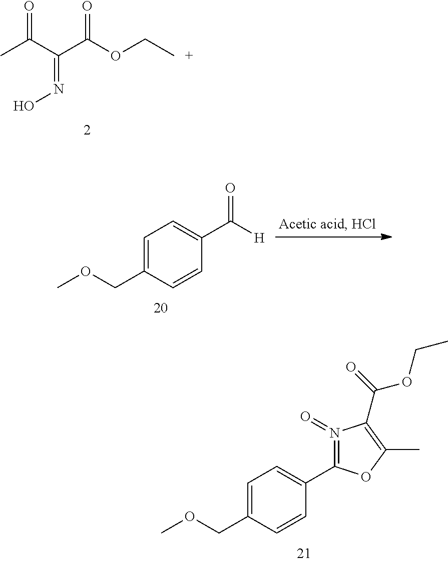

To a solution of 2-[(E)-hydroxyimino]-3-oxo-butyric acid ethyl ester (2) (1 g, 0.006 moles, 1 eq) in acetic acid (7 mL), 4-methoxymethyl-benzaldehyde (20) (1.41 g, 0.0093 moles, 1.5 eq) was added at 25° C. and the reaction mixture was cooled to 0° C. To the resulting solution, dry HCl gas was bubbled for 30 min at the same temperature (until the solution became wine red in color). The reaction mixture was gradually warmed to 25° C. and stirred for 3 h at the same temperature. The resulting reaction mixture was slowly added to vigorously stirred diethyl ether (100 mL) to obtain product 21 as a white solid, which was filtered using a Buchner funnel, and then dried under vacuum and taken to the next step without further purification (850 mg, 46%).

### Step-2

### Preparation of 2-(4-methoxymethyl-phenyl)-5-methyl-oxazole-4-carboxylic acid ethyl ester (22)

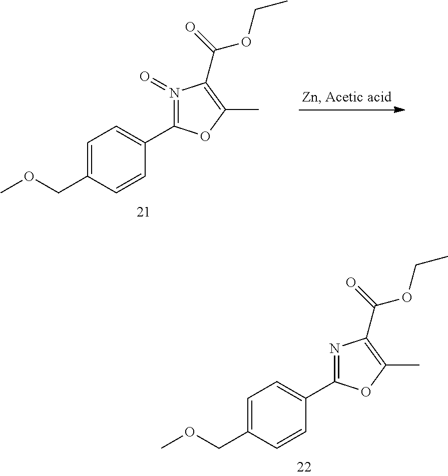

To a solution of 2-(4-methoxymethyl-phenyl)-5-methyl-3-oxy-oxazole-4-carboxylic acid ethyl ester (21) (700 mg, 2.41 mmol, 1 eq) in acetic acid (7 mL), Zn dust (470 mg, 7.21 mmol, 3 eq) was added portionwise at 0° C. The reaction mixture was allowed to stir at 25° C. for 30 min, and the resulting reaction mixture was quenched with aqueous HCl (1.5N) and filtered through a bed of Celite. The filtrate obtained was suspended between water (200 mL) and ethyl acetate (200 mL). The ethyl acetate layer was separated, dried over anhydrous sodium sulfate and concentrated under reduced pressure to obtain product 22 as a white solid (520 mg, 77%).

### Step-3

### Preparation of [2-(4-methoxymethyl-phenyl)-5-methyl-oxazol-4-yl]-methanol (23)

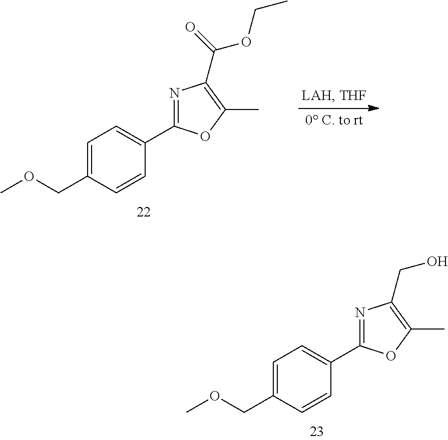

To a suspension of lithium aluminum hydride (105 mg, 2.72 mmol, 1.5 eq) in THF (10 mL) at −20° C., a solution of 2-(4-methoxymethyl-phenyl)-5-methyl-oxazole-4-carboxylic acid ethyl ester (22) (500 mg, 1.81 mmol, 1 eq) in THF (3 mL) was added dropwise, and reaction mixture was allowed to stirred at 25° C. for 30 min. The resulting reaction mixture was quenched with aqueous ammonium chloride (10%) at 0° C., diluted with ethyl acetate (150 mL), and filtered through a bed of Celite. The ethyl acetate layer was separated, dried over anhydrous sodium sulfate and concentrated under reduced pressure to obtain product 23 as a white solid (250 mg, 61%).

### Step-4

### Preparation of 4-chloromethyl-2-(4-methoxymethyl-phenyl)-5-methyl-oxazole (24)

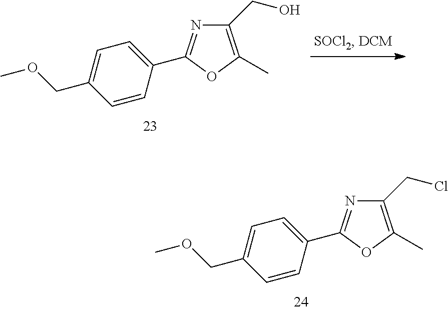 [SMILES: COCc1ccc(-c2nc(CO)c(C)o2)cc1 ; COCc1ccc(-c2nc(CCl)c(C)o2)cc1]

To a solution of [2-(4-methoxymethyl-phenyl)-5-methyl-oxazol-4-yl]-methanol (23) (1 g, 4.29 mmol, 1 eq) in DCM (10 mL) at 0° C., thionyl chloride (0.407 mL, 5.58 mmoles, 1.3 eq) was added dropwise and stirred at 25° C. for 30 min. The resulting reaction mixture was diluted with ethyl acetate (150 mL), washed with water (50 mL) and aqueous sodium bicarbonate (10%, 50 mL), dried over anhydrous sodium sulfate and concentrated under reduced pressure to obtain product 24 as an off-white solid (950 mg, 94%).

### Step-5

### Preparation of [2-(4-methoxymethyl-phenyl)-5-methyl-oxazol-4-ylmethylsulfanyl]-acetic acid ethyl ester (25)

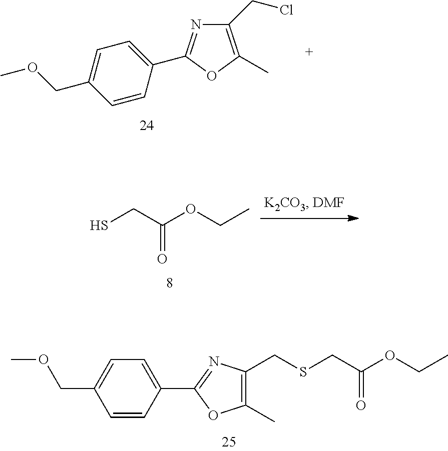

To a solution of 4-chloromethyl-2-(4-methoxymethyl-phenyl)-5-methyl-oxazole (24) (900 mg, 3.59 mmol, 1 eq) in DMF (9 mL) at 25° C., potassium carbonate (1.08 g, 7.88 mmol 2.2 eq), DMAP (catalytic) and ethyl thioacetate (8) (603 mg, 5.026 mmole, 1.4 eq) were added and stirred for 45 min at 25° C. The reaction mass was diluted with water (200 mL), extracted with ethyl acetate (2×100 mL), and the ethyl acetate layer was dried over anhydrous sodium sulfate and concentrated under reduced pressure to obtain product 25 as an off-white solid (700 mg, 58%).

### Step-6

### Preparation of [2-(4-methoxymethyl-phenyl)-5-methyl-oxazol-4-ylmethylsulfanyl]-acetic acid (26)

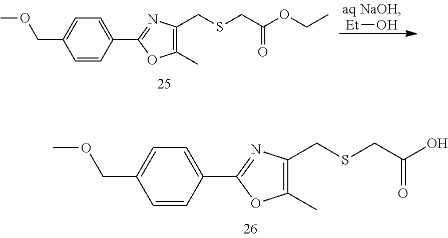

To a solution of [2-(4-methoxymethyl-phenyl)-5-methyl-oxazol-4-ylmethylsulfanyl]-acetic acid ethyl ester (25) (500 mg, 1.49 mmol, 1 eq) in methanol (5 mL) at 25° C., was added an aqueous solution of NaOH (180 mg, 4.47 mmol, 3 eq, in 3 mL) and stirred for 30 min at 25° C. The resulting reaction mixture was concentrated under reduced pressure to remove methanol, and the residue obtained was dissolved in water and acidified to pH 1 with aqueous HCl (1.5N) to obtain a white precipitate. The precipitate obtained was filtered using a Buchner funnel, dissolved in DCM (100 mL), dried over anhydrous sodium sulfate and concentrated under reduced pressure to obtain product 26 as a white solid (380 mg, 85%).

### Step-7

### Preparation of N-[2-(4-fluoro-phenyl)-ethyl]-2-[2-(4-methoxymethyl-phenyl)-5-methyl-oxazol-4-ylmethylsulfanyl]-acetamide (27)

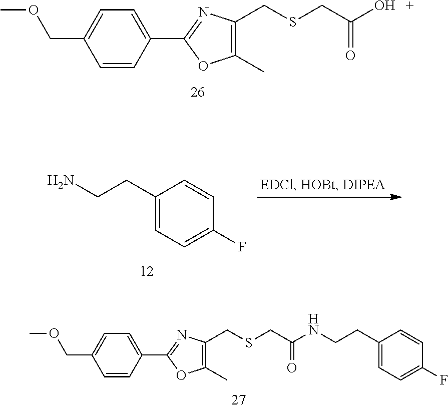

To a solution of [2-(4-methoxymethyl-phenyl)-5-methyl-oxazol-4-ylmethylsulfanyl]-acetic acid (26) (0.5 g, 1.62 mmol, 1 eq) in DMF (5 mL), DIPEA (0.525 mL, 4.04 mmol, 2.5 eq), EDCI (460 mg, 2.43 mmol, 1.5 eq) and HOBt (44 mg, 0.3254 mol, 0.2 eq) were added at 0° C. and stirred for 5 min at the same temperature. To the resulting reaction mixture at 0° C., 4-fluoro-phenethylamine (11) (250 mg, 1.78 mmol, 1.1 eq) was added and stirred for 30 min at room temperature. The reaction mixture was diluted with water (150 mL) and extracted with ethyl acetate (2×150 mL). The combined ethyl acetate layers were washed with aqueous sodium bicarbonate solution (10%, 150 mL), aqueous HCl (1.5N, 150 mL), dried over anhydrous sodium sulfate and concentrated under reduced pressure to obtain the crude product as an off-white solid. The crude product obtained upon evaporation of the solvent was purified by silica gel (230-400) column chromatography (30% ethyl acetate in hexane) to obtain pure product 27 as a white solid (300 mg, 43%).

### Step-8

### Preparation of 2-[2-(4-bromomethyl-phenyl)-5-methyl-oxazol-4-ylmethylsulfanyl]-N-[2-(4-fluoro-phenyl)-ethyl]-acetamide (28)

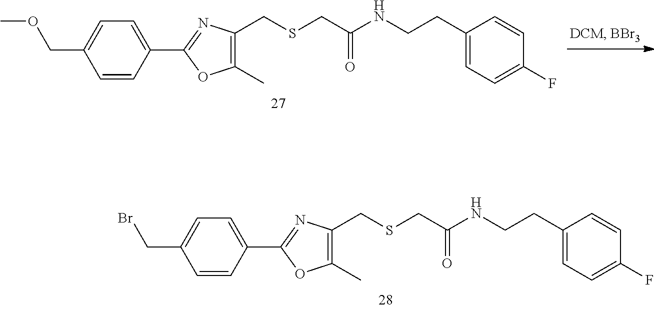 [SMILES: COCc1ccc(-c2nc(CSCC(=O)NCCc3ccc(F)cc3)c(C)o2)cc1 ; Cc1oc(-c2ccc(CBr)cc2)nc1CSCC(=O)NCCc1ccc(F)cc1]

To a solution of —N-[2-(4-fluoro-phenyl)-ethyl]-2-[2-(4-methoxymethyl-phenyl)-5-methyl-oxazol-4-ylmethylsulfanyl]-acetamide (27) (100 mg, 0.233 mmol, 1 eq) in dichloromethane (10 mL) at 0° C., boron tribromide (0.02 mL, 0.210 mmol, 0.9 eq) was added dropwise and stirred at the same temperature for 30 min. The reaction mixture was quenched with methanol (2 mL), diluted with dichloromethane (100 mL), washed with aqueous sodium bicarbonate solution (10%, 50 mL) and water (75 mL), and the organic phase was dried over sodium sulfate and concentrated under reduced pressure to obtain the crude compound as an off-white solid (110 mg). The crude product obtained upon evaporation of the solvent was purified by silica gel (230-400) column chromatography (30% ethyl acetate in hexane) to obtain pure product 28 as a white solid (90 mg, 81%).

### Step-9

### Preparation of (II-13) N-[2-(4-Fluoro-phenyl)-ethyl]-2-[5-methyl-2-(4-pyrrolidin-1-ylmethyl-phenyl)-oxazol-4-ylmethylsulfanyl]-acetamide (29)

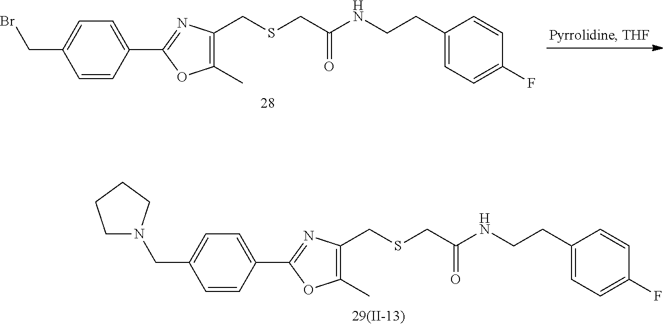 [SMILES: Cc1oc(-c2ccc(CBr)cc2)nc1CSCC(=O)NCCc1ccc(F)cc1 ; Cc1oc(-c2ccc(CN3CCCC3)cc2)nc1CSCC(=O)NCCc1ccc(F)cc1]

To a solution 2-[2-(4-bromomethyl-phenyl)-5-methyl-oxazol-4-ylmethylsulfanyl]-N-[2-(4-fluoro-phenyl)-ethyl]-acetamide (28) (90 mg, 0.189 mmol, 1 eq) in THF (10 mL), pyrrolidine (0.055 mL, 0.566 mmol, 3 eq) was added at 25° C. and stirred at the same temperature for 30 min. The reaction mixture was diluted with water (50 mL) and extracted with ethyl acetate (2×50 mL). The organic phase was separated was dried over sodium sulfate and concentrated under reduced pressure to obtain the crude product (90 mg). The crude product was further purified by preparative HPLC to obtain product 29 as an off-white solid (30 mg, 30%). MS (ESI) m/z: 468.2 (M+H)⁺

¹H NMR (300 MHz, DMSO-d₆) δ 8.04 (br s, 1H), 7.86 (d, J=8 Hz, 2H), 7.43 (d, J=8 Hz, 2H), 7.22 (m, 2H), 7.09 (t, J=9 Hz, 2H), 3.70 (s, 2H), 3.62 (s, 2H), 3.23-3.33 (m, 2H), 3.10 (s, 2H), 2.68 (t, J=7 Hz, 2H), 2.43 (br s, 4H), 2.36 (s, 3H), 1.70 (br s, 4H).

### EXAMPLE 10

The aminoalkyl analogs below were synthesized by following the experimental procedures described in Example 9.

| | MS (ESI) m/z (M + H)⁺ |
|---|---|
| 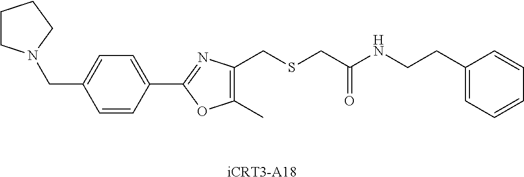 [SMILES: Cc1oc(-c2ccc(CN3CCCC3)cc2)nc1CSCC(=O)NCCc1ccccc1 ; "iCRT3-A18"] | 450.2 |
| 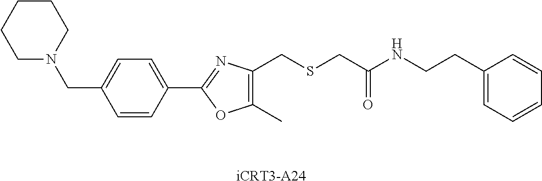 [SMILES: Cc1oc(-c2ccc(CN3CCCCC3)cc2)nc1CSCC(=O)NCCc1ccccc1 ; "iCRT3-A24"] | 464.2 |
| 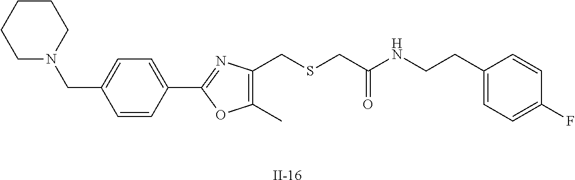 [SMILES: Cc1oc(-c2ccc(CN3CCCCC3)cc2)nc1CSCC(=O)NCCc1ccc(F)cc1 ; "II-16"] | 482.2 |
| 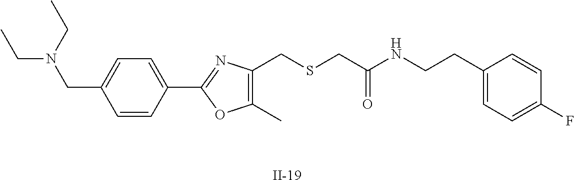 [SMILES: CCN(CC)Cc1ccc(-c2nc(CSCC(=O)NCCc3ccc(F)cc3)c(C)o2)cc1 ; "II-19"] | 470.2 |
| 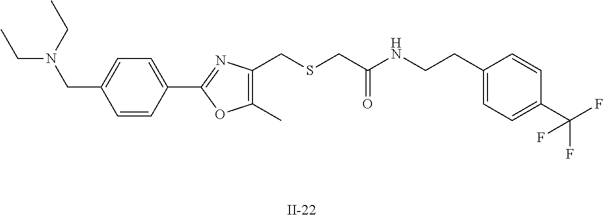 [SMILES: CCN(CC)Cc1ccc(-c2nc(CSCC(=O)NCCc3ccc(C(F)(F)F)cc3)c(C)o2)cc1 ; "II-22"] | 520.5 |
| 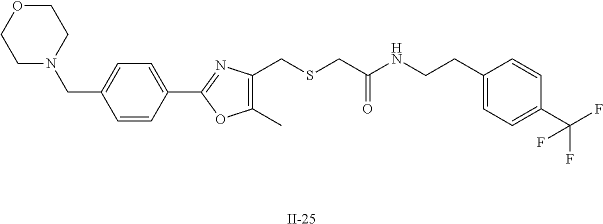 [SMILES: Cc1oc(-c2ccc(CN3CCOCC3)cc2)nc1CSCC(=O)NCCc1ccc(C(F)(F)F)cc1 ; "II-25"] | 534.5 |
| 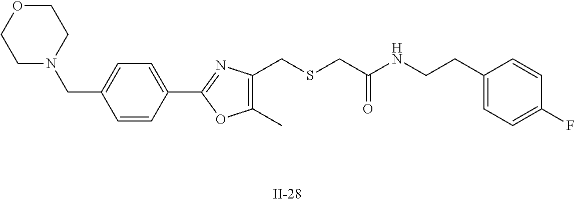 [SMILES: Cc1oc(-c2ccc(CN3CCOCC3)cc2)nc1CSCC(=O)NCCc1ccc(F)cc1 ; "II-28"] | 484.2 |
| 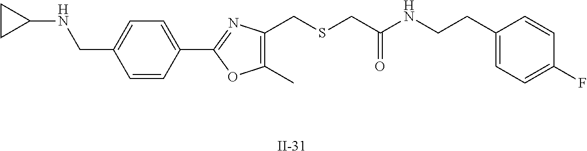 [SMILES: Cc1oc(-c2ccc(CNC3CC3)cc2)nc1CSCC(=O)NCCc1ccc(F)cc1 ; "II-31"] | 454.2 |

### EXAMPLE 11

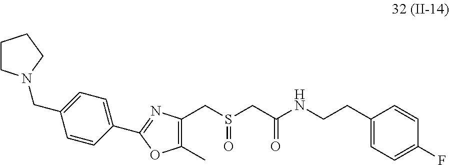 [SMILES: Cc1oc(-c2ccc(CN3CCCC3)cc2)nc1CS(=O)CC(=O)NCCc1ccc(F)cc1]

### Step-1

### Preparation of [2-(4-fluoro-phenyl)-ethyl]-2-[2-(4-methoxymethyl-phenyl)-5-methyl-oxazol-4-ylmethanesulfinyl]-acetamide (30)

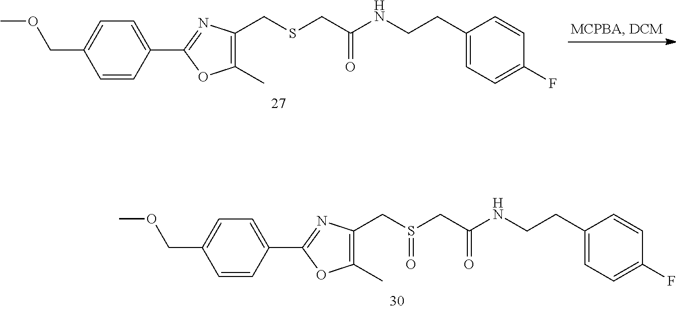 [SMILES: COCc1ccc(-c2nc(CSCC(=O)NCCc3ccc(F)cc3)c(C)o2)cc1 ; COCc1ccc(-c2nc(CS(=O)CC(=O)NCCc3ccc(F)cc3)c(C)o2)cc1]

To a solution of —N-[2-(4-fluoro-phenyl)-ethyl]-2-[2-(4-methoxymethyl-phenyl)-5-methyl-oxazol-4-ylmethylsulfanyl]-acetamide (27) (100 mg, 0.23 mmol, 1 eq) in DCM (5 mL), 3-chloroperbenzoic acid (70%) (56 mg, 0.23 mmol, 1 eq) was added and stirred at 25° C. for 30 min. The reaction mixture was diluted with DCM (100 mL), washed with aqueous sodium bicarbonate solution (10%, 50 mL) and water (75 mL), dried over sodium sulfate and concentrated under reduced pressure to obtain the crude compound as a white solid (110 mg). The crude product obtained was purified by silica gel (230-400) column chromatography (30% ethyl acetate in hexane) to obtain pure product 30 as a white solid (65 mg, 63%).

### Step-2

### Preparation of 2-[2-(4-bromomethyl-phenyl)-5-methyl-oxazol-4-ylmethanesulfinyl]-N-[2-(4-fluoro-]-acetamide (31)

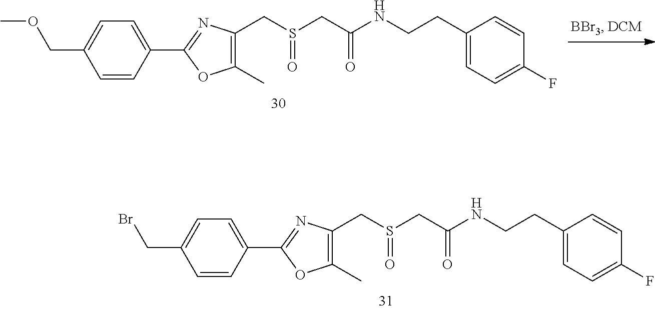 [SMILES: COCc1ccc(-c2nc(CS(=O)CC(=O)NCCc3ccc(F)cc3)c(C)o2)cc1 ; Cc1oc(-c2ccc(CBr)cc2)nc1CS(=O)CC(=O)NCCc1ccc(F)cc1]

To a solution of [2-(4-fluoro-phenyl)-ethyl]-2-[2-(4-methoxymethyl-phenyl)-5-methyl-oxazol-4-ylmethanesulfinyl]-acetamide (30) (120 mg, 0.270 mmol, 1 eq) in DCM (10 mL) at 0° C., boron tribromide (0.023 mL, 0.243 mmol, 0.9 eq) was added dropwise and stirred at the same temperature for 30 min. The reaction mixture was quenched with methanol (2 mL) at 0° C., diluted with dichloromethane (100 mL), washed with aqueous sodium bicarbonate solution (10%, 50 mL), water (75 mL), dried over anhydrous sodium sulfate and concentrated under reduced pressure to obtain the crude compound as an off-white solid (110 mg). The crude product obtained was purified by silica gel (230-400) column chromatography (30% ethyl acetate in hexane) to obtain pure product 31 as a white solid (100 mg, 75%).

### Step-3

### Preparation of N-[2-(4-fluoro-phenyl)-ethyl]-2-[5-methyl-2-(4-pyrrolidin-1-ylmethyl-phenyl)-oxazol-4-ylmethanesulfinyl]-acetamide (32/(II-14)

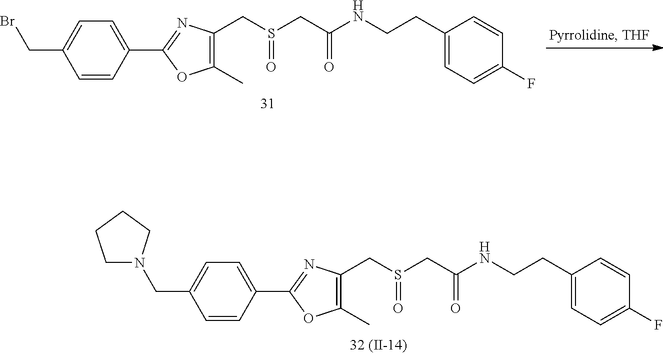 [SMILES: Cc1oc(-c2ccc(CBr)cc2)nc1CS(=O)CC(=O)NCCc1ccc(F)cc1 ; Cc1oc(-c2ccc(CN3CCCC3)cc2)nc1CS(=O)CC(=O)NCCc1ccc(F)cc1]

To a solution 2-[2-(4-bromomethyl-phenyl)-5-methyl-oxazol-4-ylmethanesulfinyl]-N-[2-(4-fluoro-]-acetamide (31) (100 mg, 0.203 mmol, 1 eq) in THF (10 mL), pyrrolidine (0.05 mL, 0.608 mmol, 3 eq) was added at 25° C. and stirred at the same temperature for 30 min. The reaction mixture was diluted with water (100 mL), extracted with ethyl acetate (2×50 mL), and the organic phase was separated, dried over sodium sulfate and concentrated under reduced pressure to obtain the crude product. The crude product was further purified with preparative HPLC to obtain product 32 as an off-white solid (30 mg, 31%).

MS (ESI) m/z: 484.5 (M+H)⁺

¹H NMR (300 MHz, DMSO-d₆) δ 8.39 (br s, 1H), 7.88 (d, J=8.1 Hz, 2H), 7.46 (d, J=8.1 Hz, 2H), 7.26 (dd, J=5.9, 8.2 Hz, 2H), 7.10 (t, J=9.0 Hz, 2H), 4.18 (d, J=14 Hz, 1H), 4.04 (d, J=14 Hz, 1H), 3.82 (d, J=13 Hz, 1H), 3.63 (s, 2H), 3.60 (d, J=13 Hz, 1H), 3.27-3.39 (m, 2H), 2.72 (m, 2H), 2.45 (br s, 4H), 2.40 (s, 3H), 1.71 (br s, 4H).

### EXAMPLE 12

The aminoalkyl analogs below were synthesized by following the experimental procedure described in Example 11.

| | MS (ESI) m/z (M + H)⁺ |
|---|---|
| 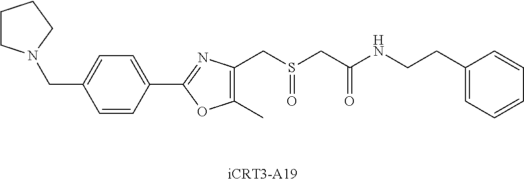 [SMILES: Cc1oc(-c2ccc(CN3CCCC3)cc2)nc1CS(=O)CC(=O)NCCc1ccccc1 ; "iCRT3-A19"] | 466.1 |
| 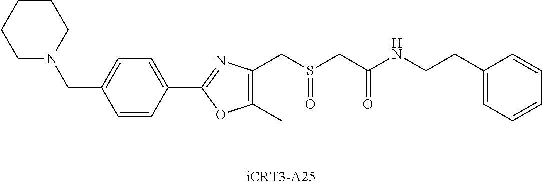 [SMILES: Cc1oc(-c2ccc(CN3CCCCC3)cc2)nc1CS(=O)CC(=O)NCCc1ccccc1 ; "iCRT3-A25"] | 480.5 |
| 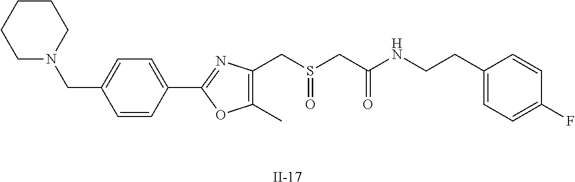 [SMILES: Cc1oc(-c2ccc(CN3CCCCC3)cc2)nc1CS(=O)CC(=O)NCCc1ccc(F)cc1 ; "II-17"] | 498.4 |
| 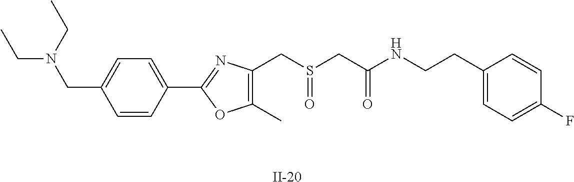 [SMILES: CCN(CC)Cc1ccc(-c2nc(CS(=O)CC(=O)NCCc3ccc(F)cc3)c(C)o2)cc1 ; "II-20"] | 486.2 |
| 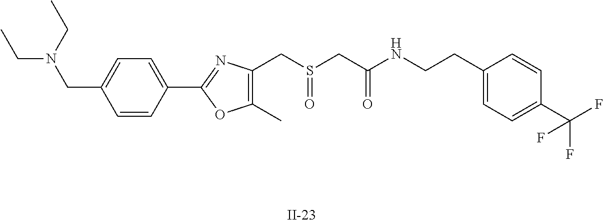 [SMILES: CCN(CC)Cc1ccc(-c2nc(CS(=O)CC(=O)NCCc3ccc(C(F)(F)F)cc3)c(C)o2)cc1 ; "II-23"] | 536.5 |
| 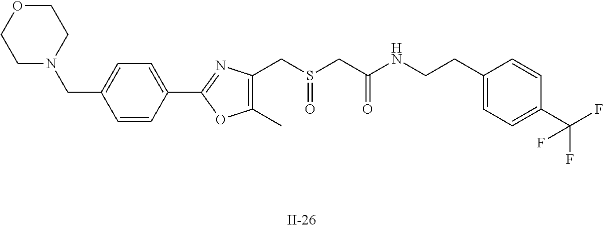 [SMILES: Cc1oc(-c2ccc(CN3CCOCC3)cc2)nc1CS(=O)CC(=O)NCCc1ccc(C(F)(F)F)cc1 ; "II-26"] | 550.5 |
| 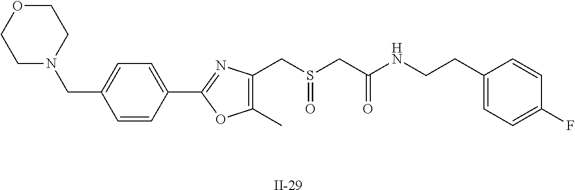 [SMILES: Cc1oc(-c2ccc(CN3CCOCC3)cc2)nc1CS(=O)CC(=O)NCCc1ccc(F)cc1 ; "II-29"] | 500.2 |
| 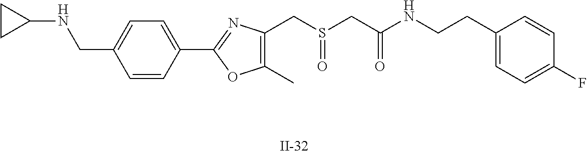 [SMILES: Cc1oc(-c2ccc(CNC3CC3)cc2)nc1CS(=O)CC(=O)NCCc1ccc(F)cc1 ; "II-32"] | 470.2 |

### EXAMPLE 13

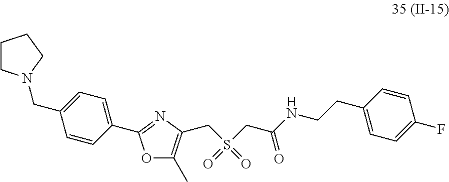 [SMILES: Cc1oc(-c2ccc(CN3CCCC3)cc2)nc1CS(=O)(=O)CC(=O)NCCc1ccc(F)cc1]

### Step 1

### Preparation of N-[2-(4-fluoro-phenyl)-ethyl]-2-[2-(4-methoxymethyl-phenyl)-5-methyl-oxazol-4-ylmethanesulfonyl]-acetamide (33)

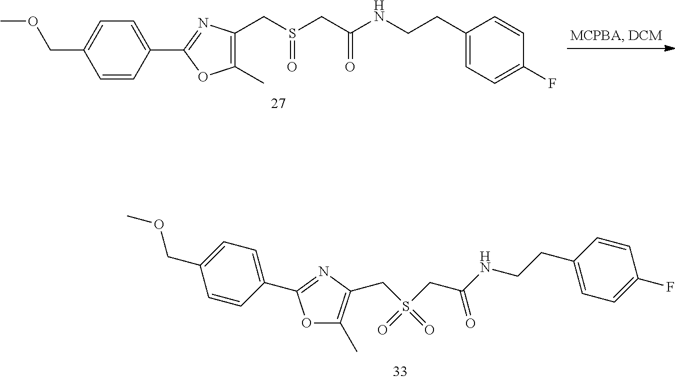 [SMILES: COCc1ccc(-c2nc(CS(=O)CC(=O)NCCc3ccc(F)cc3)c(C)o2)cc1 ; COCc1ccc(-c2nc(CS(=O)(=O)CC(=O)NCCc3ccc(F)cc3)c(C)o2)cc1]

To a solution of —N-[2-(4-fluoro-phenyl)-ethyl]-2-[2-(4-methoxymethyl-phenyl)-5-methyl-oxazol-4-ylmethylsulfanyl]-acetamide (27) (150 mg, 0.35 mmol, 1 eq) in DCM (10 mL), 3-chloroperbenzoic acid (70%) (77 mg, 0.315 mmol, 1 eq) was added at 0° C. and stirred at 25° C. for 30 min. The resulting reaction mixture was diluted with DCM (100 mL), washed with aqueous sodium bicarbonate solution (10%, 50 mL) and water (75 mL), dried over sodium sulfate and concentrated under reduced pressure to obtain the crude compound as an off-white solid (110 mg). The crude product obtained was purified by silica gel (230-400) column chromatography (30% ethyl acetate in hexane) to obtain pure product 33 as a white solid (100 mg, 62%).

### Step-2

### Preparation of 2-[2-(4-bromomethyl-phenyl)-5-methyl-oxazol-4-ylmethanesulfonyl]-N-[2-(4-fluoro-phenyl)-ethyl]-acetamide (31)

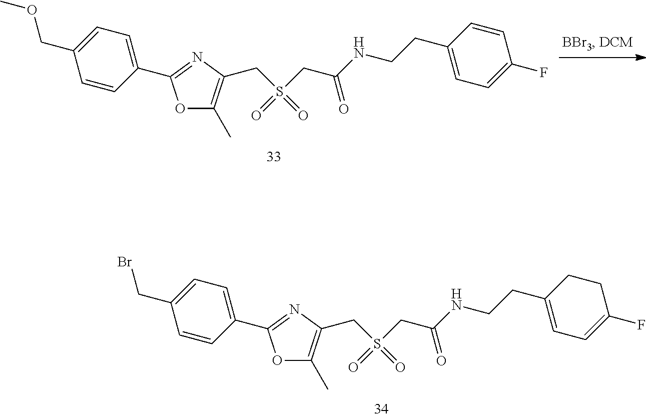 [SMILES: COCc1ccc(-c2nc(CS(=O)(=O)CC(=O)NCCc3ccc(F)cc3)c(C)o2)cc1 ; Cc1oc(-c2ccc(CBr)cc2)nc1CS(=O)(=O)CC(=O)NCCC1=CC=C(F)CC1]

To a solution of N-[2-(4-fluoro-phenyl)-ethyl]-2-[2-(4-methoxymethyl-phenyl)-5-methyl-oxazol-4-ylmethanesulfonyl]-acetamide (33) (100 mg, 0.217 mmol, 1 eq) in DCM at 0° C., boron tribromide (0.019 mL, 0.193 mmol, 0.9 eq) was added dropwise and stirred at same temperature for 30 min. The reaction mixture quenched with methanol (2 mL) at 0° C., diluted with dichloromethane (100 mL), washed with aqueous sodium bicarbonate solution (10%, 50 mL), water (75 mL), and the organic phase was dried over sodium sulfate and concentrated under reduced pressure to obtain the crude compound. The crude product obtained was purified by silica gel (230-400) column chromatography (30% ethyl acetate in hexane) to obtain pure product 34 as a white solid (90 mg, 81%).

### Step-3

### Preparation of N-[2-(4-fluoro-phenyl)-ethyl]-2-[5-methyl-2-(4-pyrrolidin-1-ylmethyl-phenyl)-oxazol-4-ylmethanesulfonyl]-acetamide (35/II-15)

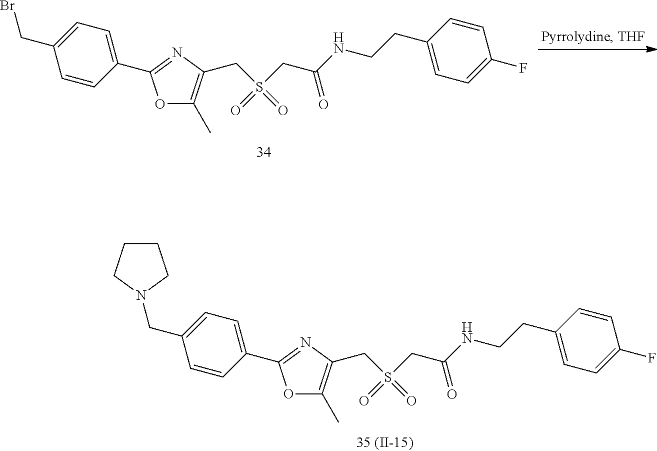 [SMILES: Cc1oc(-c2ccc(CBr)cc2)nc1CS(=O)(=O)CC(=O)NCCc1ccc(F)cc1 ; Cc1oc(-c2ccc(CN3CCCC3)cc2)nc1CS(=O)(=O)CC(=O)NCCc1ccc(F)cc1]

To a solution 2-[2-(4-bromomethyl-phenyl)-5-methyl-oxazol-4-ylmethanesulfonyl]-N-[2-(4-fluoro-phenyl)-ethyl]-acetamide (31) (90 mg, 0.177 mmol, 1 eq) in THF (10 mL), pyrrolidine (0.06 mL, 0.530 mmol, 3 eq) was added at 25° C. and stirred at the same temperature for 30 min. The resulting reaction mixture was diluted with water (100 mL) and extracted with ethyl acetate (2×75 mL), and the combined organic phases were separated, dried over sodium sulfate and concentrated under reduced pressure to obtain the crude product (90 mg). The crude product was further purified with preparative HPLC to obtain product 35 as an off-white solid (30 mg, 28%).

MS (ESI) m/z: 500.2 (M+H)⁺

¹H NMR (300 MHz, DMSO-d₆) δ 8.47 (br s, 1H), 7.88 (d, J=7.8 Hz, 2H), 7.46 (d, J=7.8 Hz, 2H), 7.29 (m, 2H), 7.12 (t, J=9.0 Hz, 2H), 4.65 (s, 2H), 4.13 (s, 2H), 3.63 (s, 2H), 3.30-3.40 (m, 2H), 2.77 (m, 2H), 2.43 (br s, 7H), 1.71 (br s, 4H).

### EXAMPLE 14

The aminoalkyl analogs below were synthesized by following the experimental procedures described in Example 13.

| | MS (ESI) m/z (M + H)⁺ |
|---|---|
| 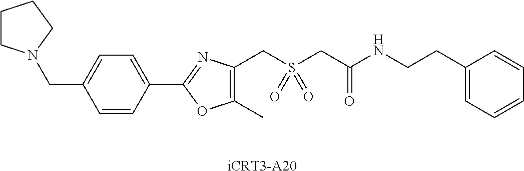 [SMILES: Cc1oc(-c2ccc(CN3CCCC3)cc2)nc1CS(=O)(=O)CC(=O)NCCc1ccccc1 ; "iCRT3-A20"] | 482.2 |
| 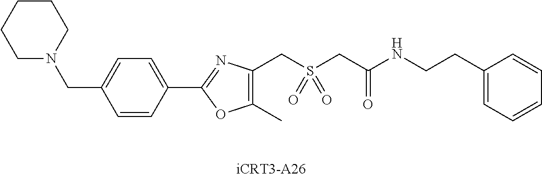 [SMILES: Cc1oc(-c2ccc(CN3CCCCC3)cc2)nc1CS(=O)(=O)CC(=O)NCCc1ccccc1 ; "iCRT3-A26"] | 496.1 |
| 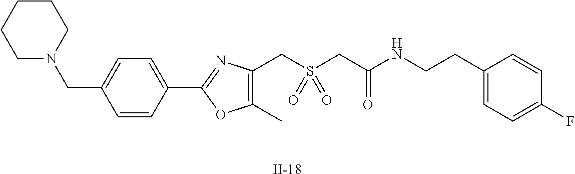 [SMILES: Cc1oc(-c2ccc(CN3CCCCC3)cc2)nc1CS(=O)(=O)CC(=O)NCCc1ccc(F)cc1 ; "II-18"] | ¹H NMR (300 MHz, DMSO-d₆) δ 8.46 (br s, 1H), 7.88 (d, J = 8.1 Hz, 2H), 7.44 (d, J = 78.1 Hz, 2H), 7.29 (m, 2H), 7.12 (t, J = 8.7 Hz, 2H), 4.65 (s, 2H), 4.13 (s, 2H), 3.48 (s, 2H), 3.30-3.40 (m, 2H), 2.75 (m, 2H), 2.43 (s, 3H), 2.25-2.37 (m, 4H), 1.31-1.56 (m, 6H). |
| 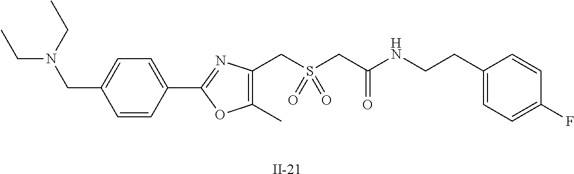 [SMILES: CCN(CC)Cc1ccc(-c2nc(CS(=O)(=O)CC(=O)NCCc3ccc(F)cc3)c(C)o2)cc1 ; "II-21"] | 502.2 |
| 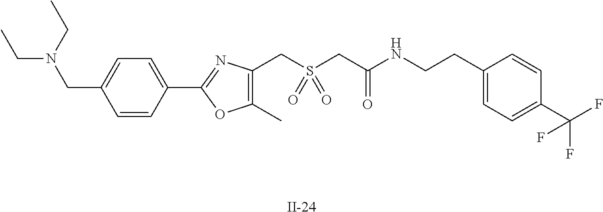 [SMILES: CCN(CC)Cc1ccc(-c2nc(CS(=O)(=O)CC(=O)NCCc3ccc(C(F)(F)F)cc3)c(C)o2)cc1 ; "II-24"] | 552.5 |
| 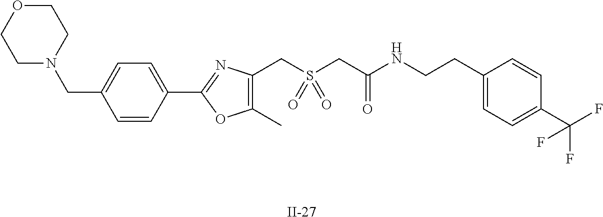 [SMILES: Cc1oc(-c2ccc(CN3CCOCC3)cc2)nc1CS(=O)(=O)CC(=O)NCCc1ccc(C(F)(F)F)cc1 ; "II-27"] | 566.5 |
| 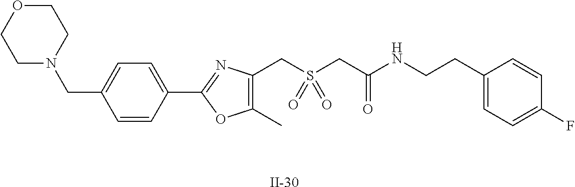 [SMILES: Cc1oc(-c2ccc(CN3CCOCC3)cc2)nc1CS(=O)(=O)CC(=O)NCCc1ccc(F)cc1 ; "II-30"] | 516.1 |
| 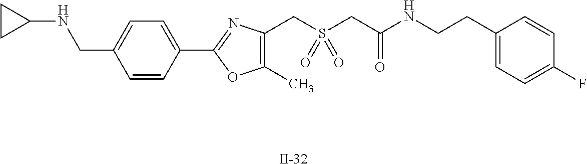 [SMILES: Cc1oc(-c2ccc(CNC3CC3)cc2)nc1CS(=O)(=O)CC(=O)NCCc1ccc(F)cc1 ; "II-32"] | 486.2 |

### EXAMPLE 15

iCRT3 Analogs Testing—Assay Protocol

### Methods and Materials

### Cell Lines and Culture:

STF and STF3A cells were cultured in DMEM, 4500 mg/L glucose supplemented with 10% fetal bovine serum, 2 mM L-glutamine, 110 mg/L sodium pyruvate and Penicillin/Streptomycin.

STF cells are HEK293 cells stably expressing the Super 8X Topflash M50 (http://www.addgene.org/12456/; the entire content of which is incorporated herein by reference) reporter. STF3A cells are HEK293 cells stably expressing both Super 8X Topflash reporter and pPGK-mWnt3A. The above cell lines are a gift from David Virshup.

L-Wnt3A cells were cultured in DMEM, 4500 mg/L glucose supplemented with 10% fetal bovine serum and 2 mM L-glutamine and 110 mg/L sodium pyruvate and Penicillin/Streptomycin. Wnt3A-Conditioned media was prepared with L-Wnt3A cells cultured according to recommended protocols from ATCC (http://www.atcc.org/products/all/CRL-2647.aspx#culturemethod).

Unless otherwise indicated, all experiments described herein that call for supplemental Wnt3a utilize Wnt3a conditioned media prepared by harvesting media from L-cells stably transfected with a Wnt3a coding construct (available from ATCC #CRL-2647). The cells are cultured in DMEM containing 10% fetal bovine serum (FBS). The medium, harvested from adherent cells cultured to about 80% confluency over 4 days, is purified through a 0.2 μm filter and stored at 4° C. over several months without an appreciable loss in activity [Willert et al. Nature 423, 448-52 (2003)].

### IC₅₀ Assay:

STF and STF3A cells were seeded in 96 well plates, with 20,000 cells in 100 μL media per well. Following 16 hours incubation, 50 μL of media per well containing STF cells were replaced with the same volume of Wnt3A-Conditioned media.

iCRT3 and analogs were tested in triplicates at a concentration range of 50 μM-0.00013 μM, at 5-fold dilutions. Cells were incubated with the compounds for 24 hours.

Cell viability was measured after incubation with 100 μL of PrestoBlue® Cell Viability Reagent in DMEM (1:9) for 30 minutes. Following fluorescence measurements, media containing PrestoBlue reagents was aspirated. Cell were lysed in 60 μL of Dual-Glo® Reagent from the Dual-Glo® Luciferase Assay System. Luciferase activity was quantified with a luminometer after 10 minutes of incubation at room temperature.

### Data Processing:

Background subtracted luminescence readings (RLU) from the Dual-Glo® Luciferase assay were normalized against background subtracted fluorescence values (RFU) from the PrestoBlue® Cell Viability assay.

The RLU/RFU values were plotted using GraphPad Prism (www.graphpad.com). The curve was fitted with a three parameter curve fitting equation: Y=Min value+(Max value−Min value)/[1+10̂(X−lg IC₅₀)].

### Results

Cell lines tested include STF, which are HEK293 cells expressing firefly luciferase under the control of the TCF promoter, and STF3A, which are the same as STF but also expresses Wnt3a constitutively. The two cell lines facilitate the testing of the compounds in the context of Wnt signaling activated in a paracrine (STF) versus autocrine (STF3A) fashion. Testing of the iCRT3 analogs revealed a significant improvement in the IC₅ₒs of the analogs, from the 14-30 μM range for parent iCRT3 to 2-8 μM range for II-1 and II-7. The data also suggests that replacement with 4-fluorophenyl, 4-chlorophenyl or pyridin-3-yl groups significantly improved inhibitory activity on the Wnt reporter without exhibiting any non-specific cytotoxicity in HEK293 cells.

### Protocol

Day 0—Seed cells in 96-well plates (20,000 cells per well)

Day 1—Add compounds. For STF cells, half of the medium was also changed to Wnt3a conditioned medium.

Day 2—Measure PrestoBlue (cell viability) and Dual-Glo luciferase (TCF promoter activity).

The concentration range for iCRT3 was 10 μM-0.0000001 μM. All other compounds were tested at a concentration range of 10 μM-0.000026 μM.

### EXAMPLE 16

### Exemplary Compounds of the Invention

The following compounds, as exemplified in Tables 1-3, have been prepared, or can be prepared according to the synthetic schemes described herein, or can be prepared according to the synthetic methods known to one skilled in the art.

TABLE 1
Oxazole amides (X = —O—)
I

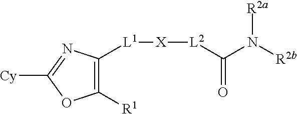

| ID | Structure | MW |
|---|---|---|
| I-1 | 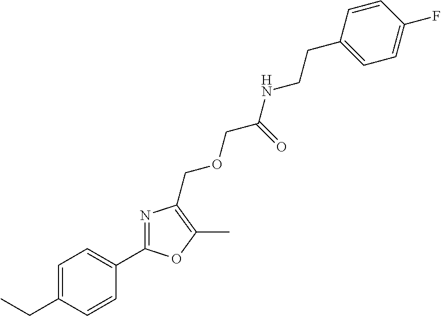 [SMILES: CCc1ccc(-c2nc(COCC(=O)NCCc3ccc(F)cc3)c(C)o2)cc1] | 396.47 |
| I-2 | 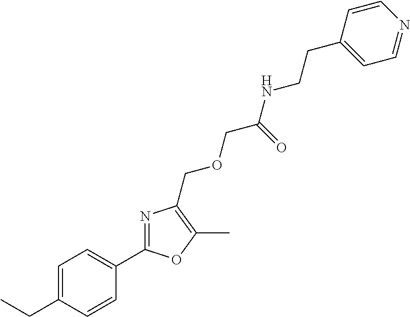 [SMILES: CCc1ccc(-c2nc(COCC(=O)NCCc3ccncc3)c(C)o2)cc1] | 379.76 |
| I-3 | 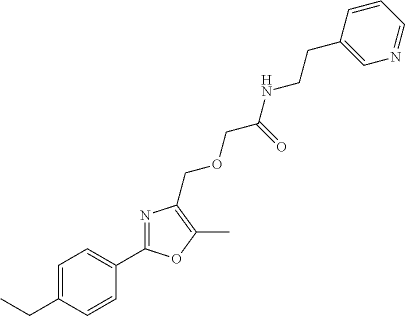 [SMILES: CCc1ccc(-c2nc(COCC(=O)NCCc3cccnc3)c(C)o2)cc1] | 379.76 |
| I-4 | 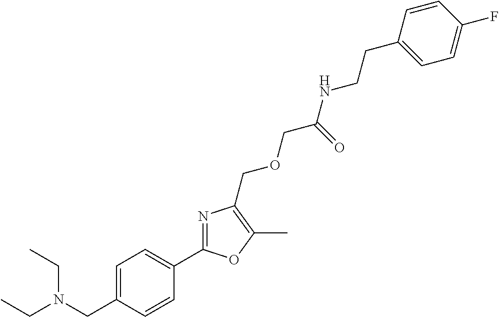 [SMILES: CCN(CC)Cc1ccc(-c2nc(COCC(=O)NCCc3ccc(F)cc3)c(C)o2)cc1] | 453.56 |
| I-5 | 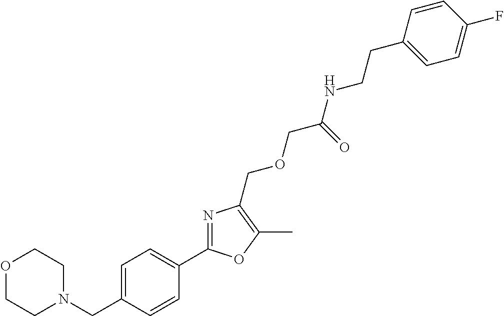 [SMILES: Cc1oc(-c2ccc(CN3CCOCC3)cc2)nc1COCC(=O)NCCc1ccc(F)cc1] | 467.55 |
| I-6 | 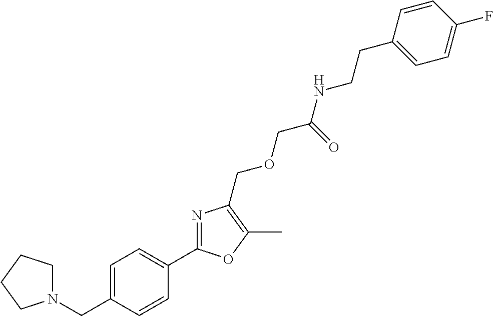 [SMILES: Cc1oc(-c2ccc(CN3CCCC3)cc2)nc1COCC(=O)NCCc1ccc(F)cc1] | 451.55 |

TABLE 2
Oxazole amides (X = —S—)
I

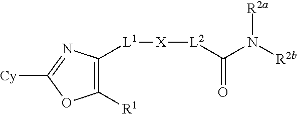

| ID | Structure | MW |
|---|---|---|
| II-1 | 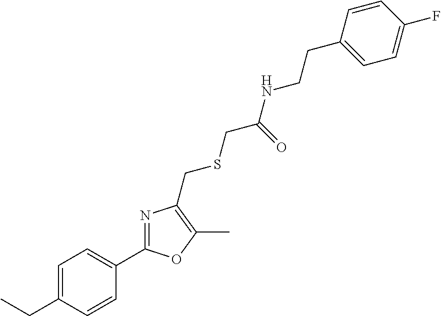 [SMILES: CCc1ccc(-c2nc(CSCC(=O)NCCc3ccc(F)cc3)c(C)o2)cc1] | 412.53 |
| II-2 | 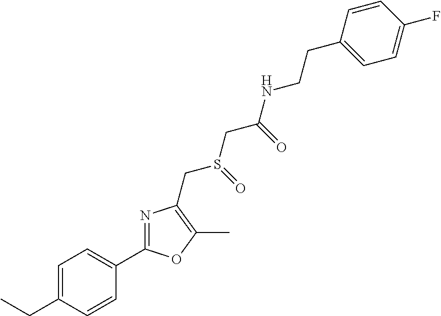 [SMILES: CCc1ccc(-c2nc(CS(=O)CC(=O)NCCc3ccc(F)cc3)c(C)o2)cc1] | 428.53 |
| II-3 | 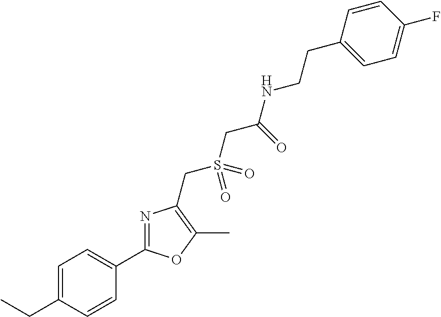 [SMILES: CCc1ccc(-c2nc(CS(=O)(=O)CC(=O)NCCc3ccc(F)cc3)c(C)o2)cc1] | 444.53 |
| II-4 | 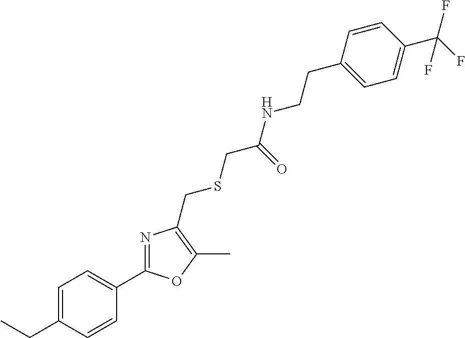 [SMILES: CCc1ccc(-c2nc(CSCC(=O)NCCc3ccc(C(F)(F)F)cc3)c(C)o2)cc1] | 462.54 |
| II-5 | 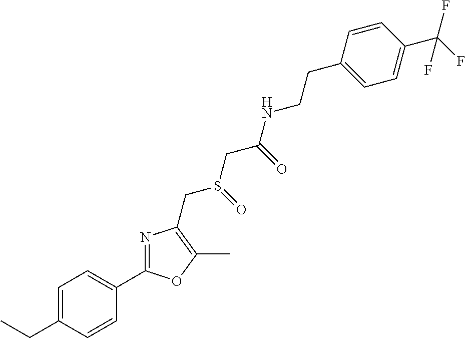 [SMILES: CCc1ccc(-c2nc(CS(=O)CC(=O)NCCc3ccc(C(F)(F)F)cc3)c(C)o2)cc1] | 478.54 |
| II-6 | 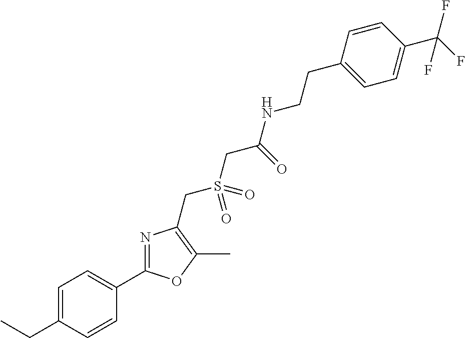 [SMILES: CCc1ccc(-c2nc(CS(=O)(=O)CC(=O)NCCc3ccc(C(F)(F)F)cc3)c(C)o2)cc1] | 494.54 |
| II-7 | 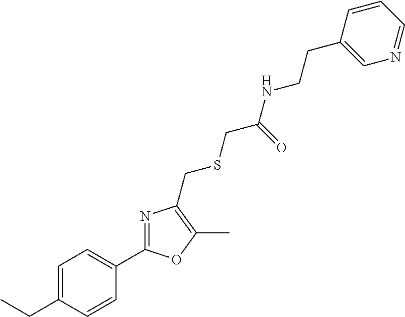 [SMILES: CCc1ccc(-c2nc(CSCC(=O)NCCc3cccnc3)c(C)o2)cc1] | 395.53 |
| II-8 | 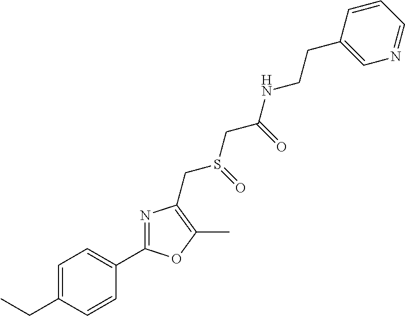 [SMILES: CCc1ccc(-c2nc(CS(=O)CC(=O)NCCc3cccnc3)c(C)o2)cc1] | 411.53 |
| II-9 | 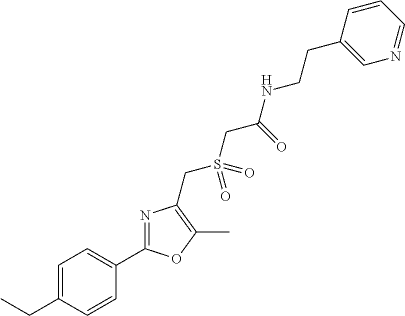 [SMILES: CCc1ccc(-c2nc(CS(=O)(=O)CC(=O)NCCc3cccnc3)c(C)o2)cc1] | 427.53 |
| II-10 | 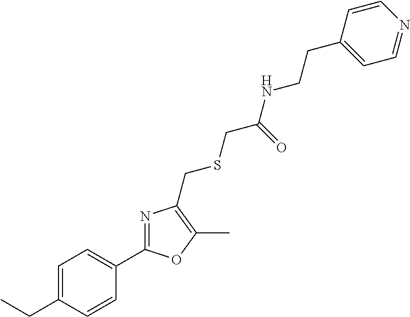 [SMILES: CCc1ccc(-c2nc(CSCC(=O)NCCc3ccncc3)c(C)o2)cc1] | 395.53 |
| II-11 | 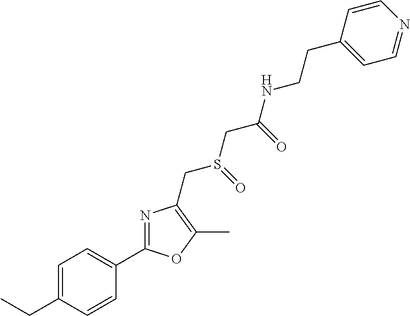 [SMILES: CCc1ccc(-c2nc(CS(=O)CC(=O)NCCc3ccncc3)c(C)o2)cc1] | 411.53 |
| II-12 | 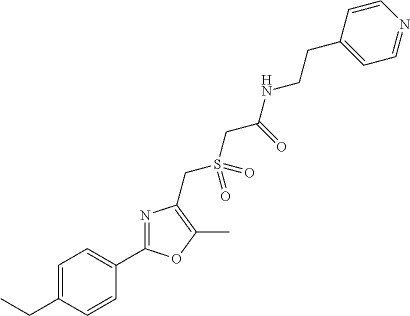 [SMILES: CCc1ccc(-c2nc(CS(=O)(=O)CC(=O)NCCc3ccncc3)c(C)o2)cc1] | 427.53 |
| II-13 | 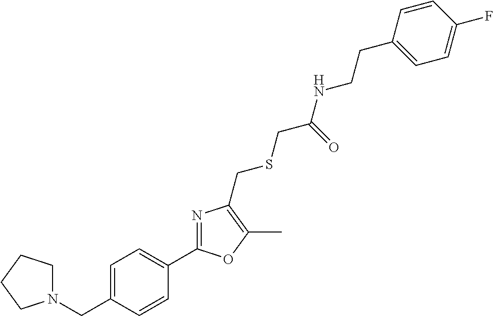 [SMILES: Cc1oc(-c2ccc(CN3CCCC3)cc2)nc1CSCC(=O)NCCc1ccc(F)cc1] | 467.61 |
| II-14 | 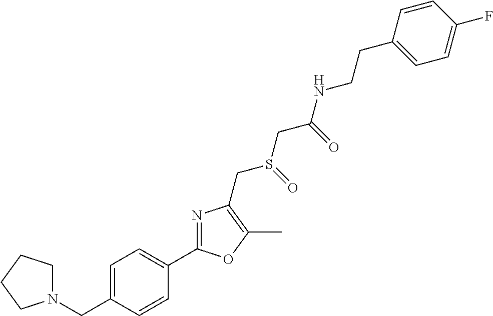 [SMILES: Cc1oc(-c2ccc(CN3CCCC3)cc2)nc1CS(=O)CC(=O)NCCc1ccc(F)cc1] | 483.61 |
| II-15 | 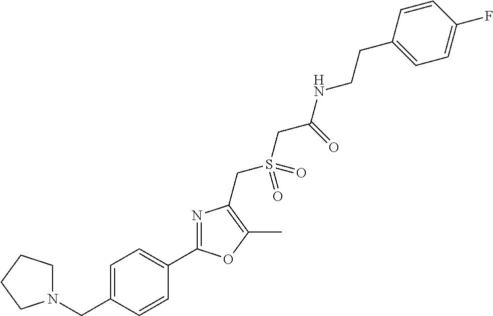 [SMILES: Cc1oc(-c2ccc(CN3CCCC3)cc2)nc1CS(=O)(=O)CC(=O)NCCc1ccc(F)cc1] | 499.61 |
| II-16 | 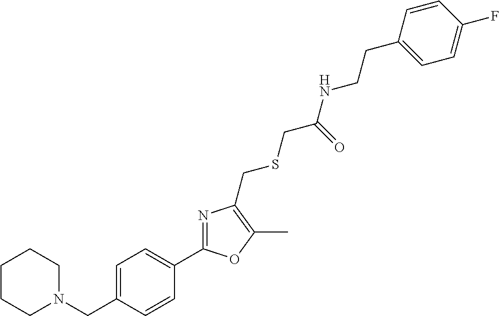 [SMILES: Cc1oc(-c2ccc(CN3CCCCC3)cc2)nc1CSCC(=O)NCCc1ccc(F)cc1] | 481.64 |
| II-17 | 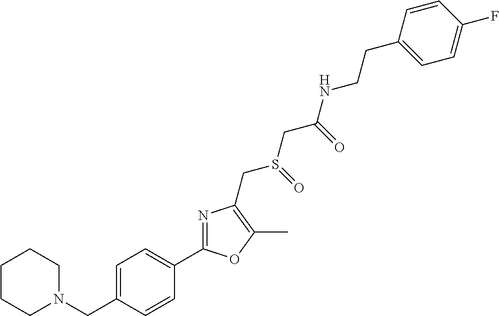 [SMILES: Cc1oc(-c2ccc(CN3CCCCC3)cc2)nc1CS(=O)CC(=O)NCCc1ccc(F)cc1] | 497.64 |
| II-18 | 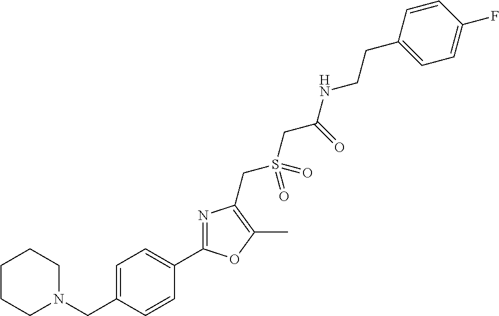 [SMILES: Cc1oc(-c2ccc(CN3CCCCC3)cc2)nc1CS(=O)(=O)CC(=O)NCCc1ccc(F)cc1] | 513.64 |
| II-19 | 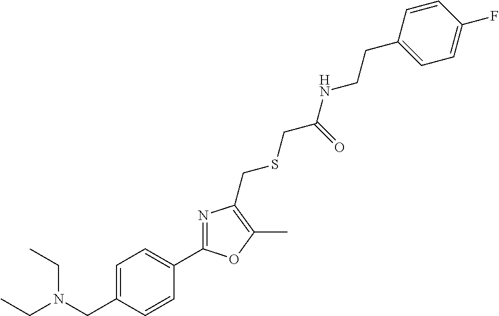 [SMILES: CCN(CC)Cc1ccc(-c2nc(CSCC(=O)NCCc3ccc(F)cc3)c(C)o2)cc1] | 469.63 |
| II-20 | 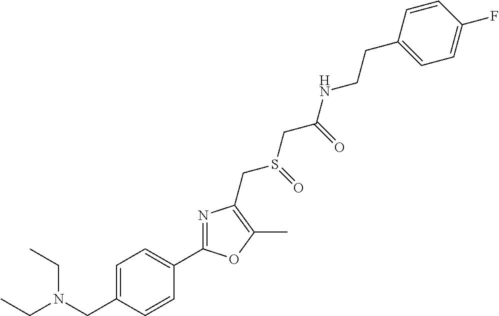 [SMILES: CCN(CC)Cc1ccc(-c2nc(CS(=O)CC(=O)NCCc3ccc(F)cc3)c(C)o2)cc1] | 485.63 |
| II-21 | 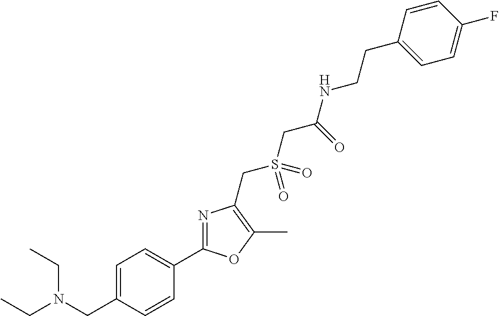 [SMILES: CCN(CC)Cc1ccc(-c2nc(CS(=O)(=O)CC(=O)NCCc3ccc(F)cc3)c(C)o2)cc1] | 501.63 |
| II-22 | 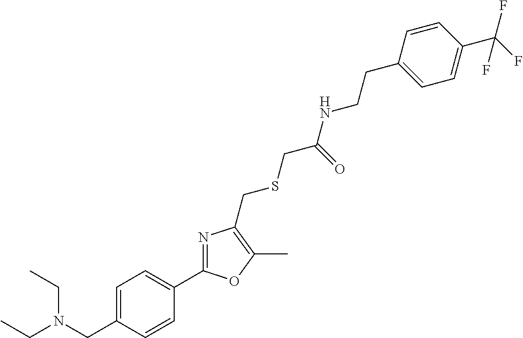 [SMILES: CCN(CC)Cc1ccc(-c2nc(CSCC(=O)NCCc3ccc(C(F)(F)F)cc3)c(C)o2)cc1] | 519.63 |
| II-23 | 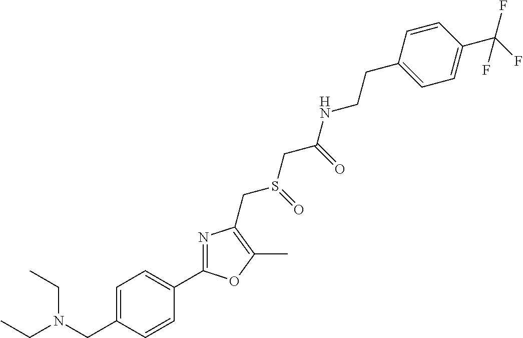 [SMILES: CCN(CC)Cc1ccc(-c2nc(CS(=O)CC(=O)NCCc3ccc(C(F)(F)F)cc3)c(C)o2)cc1] | 535.63 |
| II-24 | 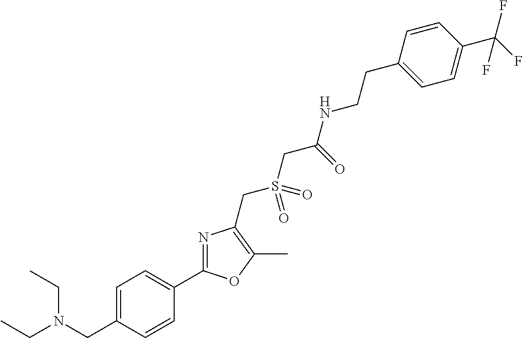 [SMILES: CCN(CC)Cc1ccc(-c2nc(CS(=O)(=O)CC(=O)NCCc3ccc(C(F)(F)F)cc3)c(C)o2)cc1] | 551.63 |
| II-25 | 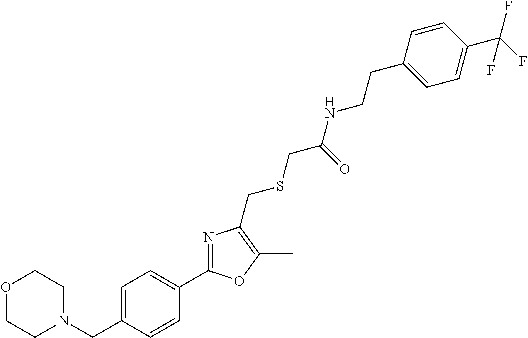 [SMILES: Cc1oc(-c2ccc(CN3CCOCC3)cc2)nc1CSCC(=O)NCCc1ccc(C(F)(F)F)cc1] | 533.62 |
| II-26 | 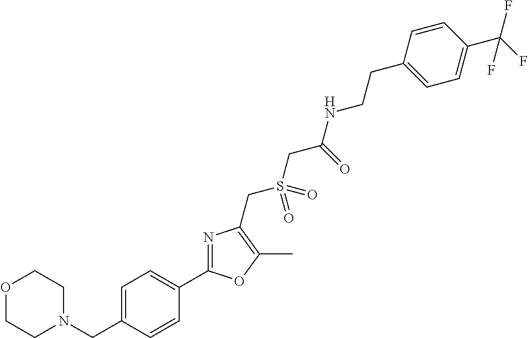 [SMILES: Cc1oc(-c2ccc(CN3CCOCC3)cc2)nc1CS(=O)(=O)CC(=O)NCCc1ccc(C(F)(F)F)cc1] | 549.62 |
| II-27 | 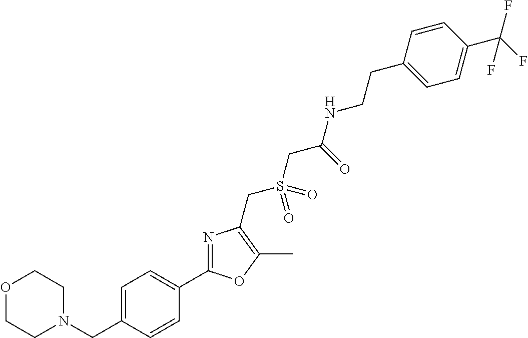 [SMILES: Cc1oc(-c2ccc(CN3CCOCC3)cc2)nc1CS(=O)(=O)CC(=O)NCCc1ccc(C(F)(F)F)cc1] | 565.62 |
| II-28 | 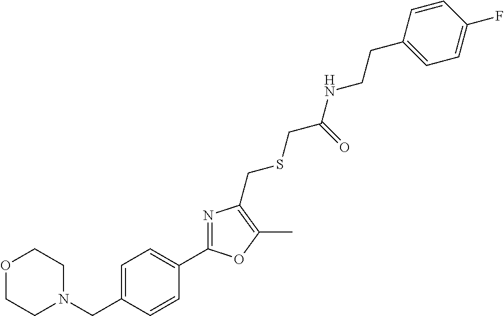 [SMILES: Cc1oc(-c2ccc(CN3CCOCC3)cc2)nc1CSCC(=O)NCCc1ccc(F)cc1] | 483.61 |
| II-29 | 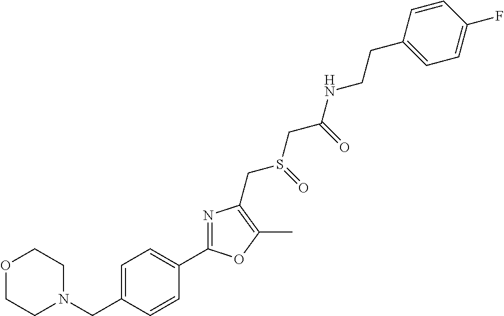 [SMILES: Cc1oc(-c2ccc(CN3CCOCC3)cc2)nc1CS(=O)CC(=O)NCCc1ccc(F)cc1] | 499.61 |
| II-30 | 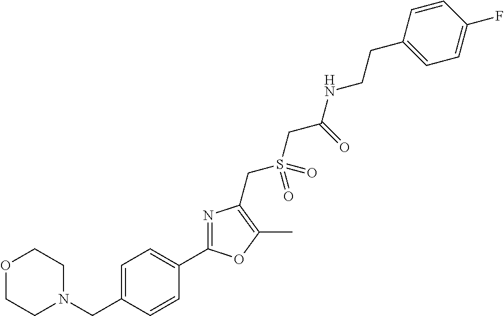 [SMILES: Cc1oc(-c2ccc(CN3CCOCC3)cc2)nc1CS(=O)(=O)CC(=O)NCCc1ccc(F)cc1] | 515.61 |
| II-31 | 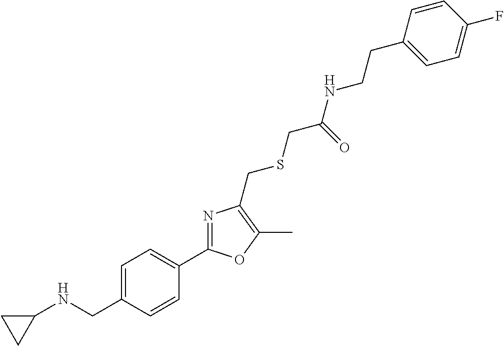 [SMILES: Cc1oc(-c2ccc(CNC3CC3)cc2)nc1CSCC(=O)NCCc1ccc(F)cc1] | 453.58 |
| II-32 | 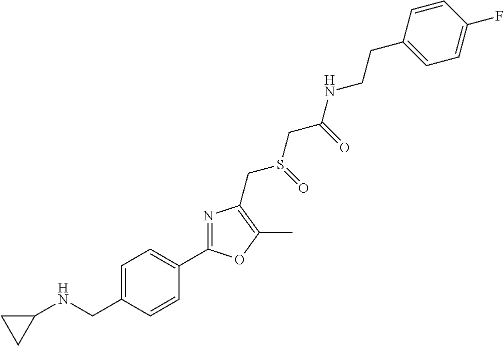 [SMILES: Cc1oc(-c2ccc(CNC3CC3)cc2)nc1CS(=O)CC(=O)NCCc1ccc(F)cc1] | 469.58 |
| II-33 | 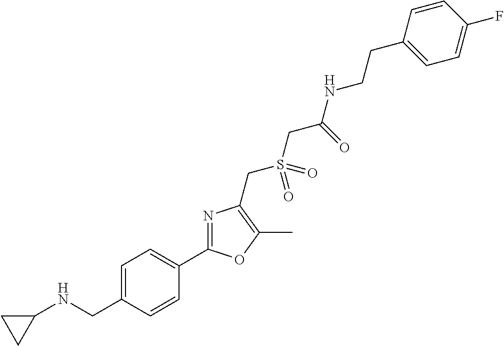 [SMILES: Cc1oc(-c2ccc(CNC3CC3)cc2)nc1CS(=O)(=O)CC(=O)NCCc1ccc(F)cc1] | 485.58 |
| II-34 | 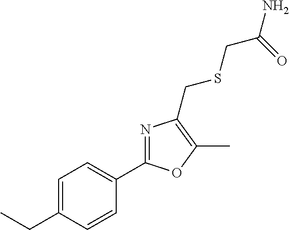 [SMILES: CCc1ccc(-c2nc(CSCC(N)=O)c(C)o2)cc1] | 290.38 |

TABLE 3
Oxazole acids (X = —S—)
IA

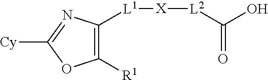

| ID | Structure | MW |
|---|---|---|
| III-1 | 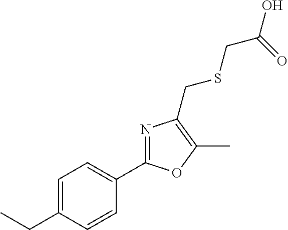 [SMILES: CCc1ccc(-c2nc(CSCC(=O)O)c(C)o2)cc1] | 291.37 |

From the foregoing description, various modifications and changes in the compositions and methods of this invention will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

At least some of the chemical names of compounds of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

Chemical structures shown herein were prepared using either ChemDraw® or ISIS®/DRAW. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

What is claimed is:

1. A method for preventing, treating or ameliorating in a mammal a disease or condition that is causally related to the aberrant activity of the Wnt signaling pathway in vivo, which comprises administering to the mammal an effective disease-treating or condition-treating amount of a compound according to formula IA: 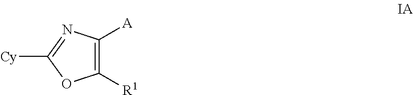
wherein
A is -L¹-X-L²—C(O)—NR^{2a}R^{2b} or -L¹-X-L²—C(O)—OR^{2a};
Cy is substituted or unsubstituted aryl or substituted or unsubstituted heteroaryl;
each of L¹ and L² is independently substituted or unsubstituted C₁-C₇ alkylene or heteroalkylene;
R¹ is hydrogen, halo, or substituted or unsubstituted C₁-C₆ alkyl;
each R^{2a} and R^{2b} is independently selected from H, substituted or unsubstituted alkyl, substituted or unsubstituted cycloalkyl, substituted or unsubstituted aryl, and substituted or unsubstituted heteroaryl; or R^{2a} and R^{2b} are joined together to form a heterocycloalkyl or heteroaryl ring;
X is —O—; or X is S, SO or SO₂;
provided that when A is -L¹-X-L²-C(O)—NR^{2a}R^{2b}, and X is S, SO or SO₂; then R^{2a} is H and R^{2b} is ethylene substituted with fluorophenyl, trifluorophenyl, or pyridyl;
or a pharmaceutically acceptable salt, solvate or prodrug thereof;
and stereoisomers, isotopic variants and tautomers thereof.

2. A compound according to formula I: 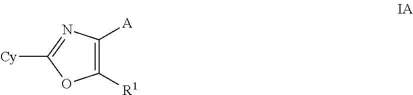
wherein
A is -L¹-X-L²—C(O)—NR^{2a}R^{2b} or -L¹-X-L²—C(O)—OR^{2a};
Cy is substituted or unsubstituted aryl or substituted or unsubstituted heteroaryl;
each of L¹ and L² is independently substituted or unsubstituted C₁-C₇ alkylene or heteroalkylene;
R¹ is hydrogen, halo, or substituted or unsubstituted C₁-C₆ alkyl;
each R^{2a} and R^{2b} is independently selected from H, substituted or unsubstituted alkyl, substituted or unsubstituted cycloalkyl, substituted or unsubstituted aryl, and substituted or unsubstituted heteroaryl; or R^{2a} and R^{2b} are joined together to form a heterocycloalkyl or heteroaryl ring;
X is —O—; or X is S, SO or SO₂;
provided that when A is -L¹-X-L²-C(O)—NR^{2a}R^{2b}, and X is S, SO or SO₂; then R^{2a} is H and R^{2b} is ethylene substituted with fluorophenyl, trifluorophenyl, or pyridyl;
or a pharmaceutically acceptable salt, solvate or prodrug thereof;
and stereoisomers, isotopic variants and tautomers thereof.

3. The method of claim 1 or the compound of claim 2, wherein A is -L¹-X-L²-C(O)—NR^{2a}R^{2b}.

4. The method of claim 1 or the compound of claim 2, wherein A is -L¹-X-L²—C(O)—OR^{2a}.

5. The method of claim 1 or the compound of claim 2, wherein the compound is according to formula I or I′: 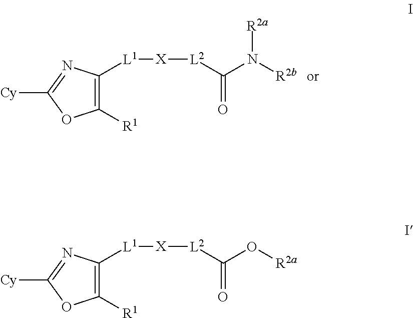
wherein
wherein Cy, L¹, X, L², R¹, R^{2a}, and R^{2b} are as in claim 1;
provided that when the compound is according to formula I, X is S, SO or SO₂, then R^{2a} is H and R^{2b} is ethylene substituted with fluorophenyl, trifluorophenyl, or pyridyl;
or a pharmaceutically acceptable salt, solvate or prodrug thereof;
and stereoisomers, isotopic variants and tautomers thereof.

6. The method of claim 1, wherein the compound is according to formula I: 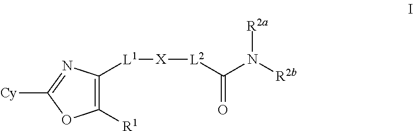
wherein
Cy is substituted or unsubstituted aryl or substituted or unsubstituted heteroaryl;
each of L¹ and L² is independently substituted or unsubstituted C₁-C₇ alkylene or heteroalkylene;
R¹ is hydrogen, halo, or substituted or unsubstituted C₁-C₆ alkyl;
each R^{2a} and R^{2b} is independently selected from H, substituted or unsubstituted alkyl, substituted or unsubstituted cycloalkyl, substituted or unsubstituted aryl, and substituted or unsubstituted heteroaryl; or R^{2a} and R^{2b} are joined together to form a heterocycloalkyl or heteroaryl ring;
X is —O—; or X is S, SO or SO₂;
provided that when X is S, SO or SO₂, then R^{2a} is H and R^{2b} is ethylene substituted with fluorophenyl, trifluorophenyl, or pyridyl;
or a pharmaceutically acceptable salt, solvate or prodrug thereof;
and stereoisomers, isotopic variants and tautomers thereof.

7. The compound of claim 2, wherein the compound is according to formula I: 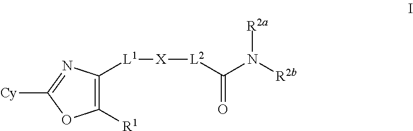
wherein
Cy is substituted or unsubstituted aryl or substituted or unsubstituted heteroaryl;
each of L¹ and L² is independently substituted or unsubstituted C₁-C₇ alkylene or heteroalkylene;
R¹ is hydrogen, halo, or substituted or unsubstituted C₁-C₆ alkyl;
each R^{2a} and R^{2b} is independently selected from H, substituted or unsubstituted alkyl, substituted or unsubstituted cycloalkyl, substituted or unsubstituted aryl, and substituted or unsubstituted heteroaryl; or R^{2a} and R^{2b} are joined together to form a heterocycloalkyl or heteroaryl ring;
X is —O—; or X is S, SO or SO₂;
provided that when X is S, SO or SO₂, then R^{2a} is H and R^{2b} is ethylene substituted with fluorophenyl, trifluorophenyl, or pyridyl;
or a pharmaceutically acceptable salt, solvate or prodrug thereof;
and stereoisomers, isotopic variants and tautomers thereof.

8. The method or the compound according to any one of claims 1-7, wherein L¹ is methylene, ethylene, propylene, or butylene, each of which unsubstituted or substituted with one or more groups selected from C₁-C₄ alkyl, halo, and hydroxyl.

9. The method or the compound according to any one of claims 1-7, wherein L¹ is —CH₂—, —CH₂—CH₂—, —CH₂—CH₂—CH₂—, or —CH₂—CH₂—CH₂—CH₂—.

10. The method or the compound according to any one of claims 1-7, wherein L¹ is —CH₂—

11. The method or the compound according to any one of claims 1-7, wherein L² is methylene, ethylene, propylene, or butylene, each of which unsubstituted or substituted with one or more groups selected from C₁-C₄ alkyl, halo, and hydroxyl.

12. The method or the compound according to any one of claims 1-7, wherein L² is —CH₂—, —CH₂—CH₂—, —CH₂—CH₂—CH₂—, or —CH₂—CH₂—CH₂—CH₂—.

13. The method or the compound according to any one of claims 1-7, wherein L² is —CH₂—.

14. The method or the compound according to any one of claims 1-13, wherein Cy is substituted or unsubstituted aryl.

15. The method or the compound according to any one of claims 1-13, wherein Cy is substituted or unsubstituted phenyl.

16. The method or the compound according to any one of claims 1-13, wherein Cy is substituted or unsubstituted naphthyl.

17. The method or the compound according to any one of claims 1-13, wherein Cy is substituted or unsubstituted heteroaryl.

18. The method or the compound according to any one of claims 1-13, wherein Cy is substituted or unsubstituted pyridyl.

19. The method or the compound according to any one of claims 1-13, wherein Cy is substituted or unsubstituted pyrimidinyl.

20. The method or the compound according to any one of claims 1-13, wherein X is —O—.

21. The method according to claim 1 or the compound according to claim 2, wherein the compound is according to formula II or II′: 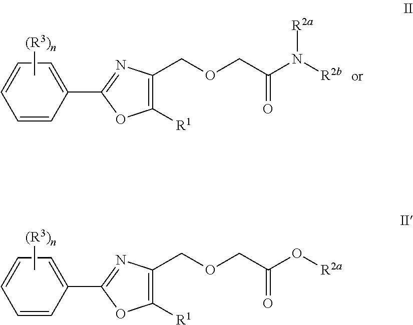
or a pharmaceutically acceptable salt, solvate or prodrug thereof;
and stereoisomers, isotopic variants and tautomers thereof;
and wherein R¹, R^{2a}, and R^{2b} are as in claim 1; n is 1, 2, 3, 4, or 5; and
each R³ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

22. The method or the compound according to any one of claims 1-21, wherein R¹ is H or substituted or unsubstituted C₁-C₆ alkyl.

23. The method or the compound according to any one of claims 1-21, wherein R¹ is halo.

24. The method or the compound according to any one of claims 1-21, wherein R¹ is Me.

25. The method according to claim 1 or the compound according to claim 2, wherein the compound is according to formula III or III′: 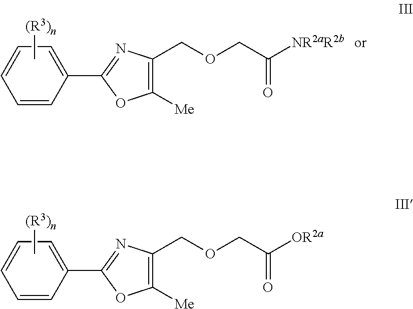
or a pharmaceutically acceptable salt, solvate or prodrug thereof;
and stereoisomers, isotopic variants and tautomers thereof;
wherein R^{2a} and R^{2b} as in claim 1; and n and R³ are as in claim 21.

26. The method or the compound according to any one of claims 1-25, wherein each of R³ is H.

27. The method or the compound according to any one of claims 1-25, wherein n, is 1; and R³ is alkyl, alkoxy, haloalkyl, or halo.

28. The method or the compound according to any one of claims 1-25, wherein n, is 1 or 2; and R³ is Me, Et, i-Pr, OMe, OEt, O-i-Pr, Cl, or F.

29. The method or the compound according to any one of claims 1-25, wherein n, is 1 or 2; and R³ is Me, OMe, SMe, or Et.

30. The method or the compound according to any one of claims 1-25, wherein n is 1; and R³ is Me.

31. The method or the compound according to any one of claims 1-25, wherein n is 1; and R³ is Et.

32. The method or the compound according to any one of claims 1-31, wherein R^{2a} is H.

33. The method or the compound according to any one of claims 1-31, wherein R^{2a} is substituted or unsubstituted alkyl.

34. The method or the compound according to any one of claims 1-31, wherein R^{2a} is substituted or unsubstituted benzyl.

35. The method or the compound according to any one of claims 1-31, wherein R^{2a} is substituted or unsubstituted phenethyl.

36. The method or the compound according to any one of claims 1-31, wherein R^{2a} is substituted or unsubstituted cycloalkyl.

37. The method or the compound according to any one of claims 1-31, wherein R^{2a} is cyclopropyl.

38. The method or the compound according to any one of claims 1-37, wherein R^{2b} is substituted or unsubstituted heteroaryl.

39. The method or the compound according to any one of claims 1-37, wherein R^{2b} is substituted or unsubstituted heterocycloalkyl.

40. The method or the compound according to any one of claims 1-31, wherein each of R^{2a} and R^{2b} is H.

41. The method or the compound according to any one of claims 1-31, wherein one of R^{2a} and R^{2b} is substituted or unsubstituted alkyl and the other is H.

42. The method or the compound according to any one of claims 1-31, wherein one of R^{2a} and R^{2b} is substituted or unsubstituted benzyl and the other is H.

43. The method or the compound according to any one of claims 1-31, wherein one of R^{2a} and R^{2b} is substituted or unsubstituted phenethyl and the other is H.

44. The method or the compound according to any one of claims 1-31, wherein one of R^{2a} and R^{2b} is substituted or unsubstituted cycloalkyl and the other is H.

45. The method or the compound according to any one of claims 1-31, wherein one of R^{2a} and R^{2b} is substituted or unsubstituted cyclopropyl and the other is H.

46. The method or the compound according to any one of claims 1-31, wherein one of R^{2a} and R^{2b} is substituted or unsubstituted cyclopentyl or cyclobutyl and the other is H.

47. The method or the compound according to any one of claims 1-31, wherein one of R^{2a} and R^{2b} is alkyl substituted with aryl, heteroaryl, cycloalkyl or heterocycloalkyl; and the other is H.

48. The method or the compound according to any one of claims 1-31, wherein one of R^{2a} and R^{2b} is methyl, ethyl, or n-propyl substituted with phenyl, pyridyl, cycloalkyl or heterocycloalkyl; and the other is H.

49. The method or the compound according to any one of claims 1-31, wherein one of R^{2a} and R^{2b} is methyl, ethyl, or n-propyl substituted with phenyl, pyridyl, cyclopropyl, cyclohexyl, cyclopentyl, cyclobutyl, piperidinyl, morphlinyl or piperazinyl; and the other is H.

50. The method or the compound according to any one of claims 1-31, wherein one of R^{2a} and R^{2b} is ethyl substituted with phenyl, pyridyl, cyclopropyl, cyclohexyl, cyclopentyl, cyclobutyl, piperidinyl, morphlinyl or piperazinyl; and the other is H.

51. The method or the compound according to any one of claims 1-31, wherein one of R^{2a} and R^{2b} is phenylethylene, pyridylethylene, cyclopropylethylene, cyclohexylethylene, cyclopentylethylene, cyclobutylethylene, piperidinylethylene, morphlinylethylene, or piperazinylethylene; and the other is H.

52. The method or the compound according to any one of claims 1-27, wherein each of phenyl, pyridyl, cyclopropyl, cyclohexyl, cyclopentyl, cyclobutyl, piperidinyl, morphlinyl or piperazinyl is substituted or unsubstituted.

53. The method or the compound according to any one of claims 48-52, wherein each of phenyl, pyridyl, cyclopropyl, cyclohexyl, cyclopentyl, cyclobutyl, piperidinyl, morphlinyl or piperazinyl is substituted with alkyl, halo or CN.

54. The method or the compound according to any one of claims 1-31, wherein R^{2a} and R^{2b} join together to form a heterocycloalkyl or heteroaryl ring.

55. The method or the compound according to any one of claims 1-31, wherein NR^{2a}R^{2b} is: 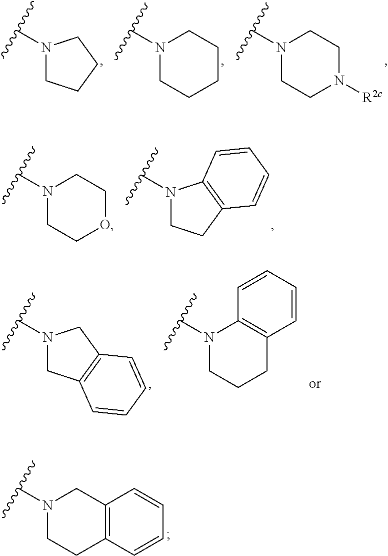
and wherein R^{2c} is H or alkyl.

56. The method according to claim 1 or the compound according to claim 2, wherein the compound is according to formula IVa, IVb, IVc or IVd: 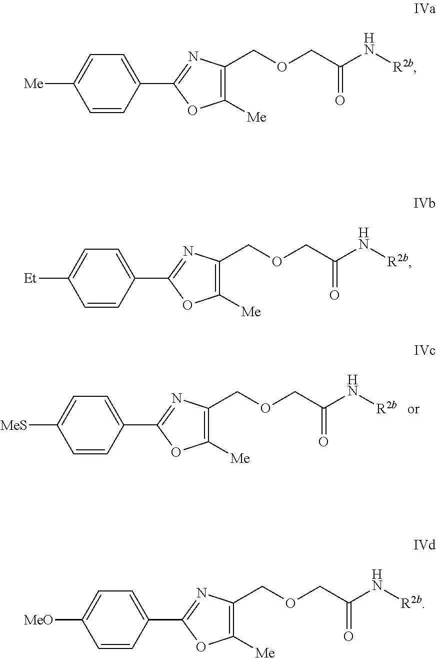
or a pharmaceutically acceptable salt, solvate or prodrug thereof;
and stereoisomers, isotopic variants and tautomers thereof;
wherein R^{2b} is as in claim 1.

57. The method or the compound according to claim 56, wherein R^{2b} is substituted or unsubstituted cycloalkyl, substituted or unsubstituted phenyl, substituted or unsubstituted benzyl, or substituted or unsubstituted phenethyl.

58. The method or the compound according to claim 56, wherein R^{2b} is substituted or unsubstituted heteroaryl, or substituted or unsubstituted heterocycloalkyl.

59. The method according to claim 1 or the compound according to claim 2, wherein the compound is according to formula IVa′, IVb′, IVc′ or IVd′: 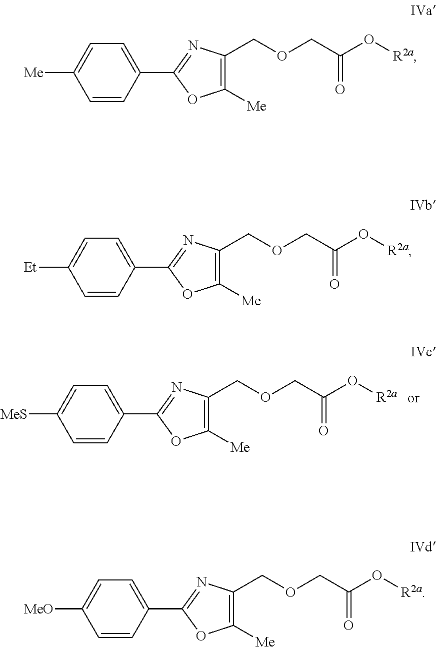
or a pharmaceutically acceptable salt, solvate or prodrug thereof;
and stereoisomers, isotopic variants and tautomers thereof;
wherein R^{2a} is as in claim 1.

60. The method or the compound according to claim 59, wherein R^{2a} is H, substituted or unsubstituted cycloalkyl, substituted or unsubstituted phenyl, substituted or unsubstituted benzyl, or substituted or unsubstituted phenethyl.

61. The method or the compound according to claim 59, wherein R^{2a} is substituted or unsubstituted heteroaryl, or substituted or unsubstituted heterocycloalkyl.

62. The method or the compound according to claim 59, wherein R^{2a} is H, Me, Et, n-Pr, i-Pr, n-Bu, i-Bu, sec-Bu, t-Bu, or cyclopropyl.

63. The method according to claim 1 or the compound according to claim 2, wherein the compound is according to formula Va, Vb, Vc, or Vd: 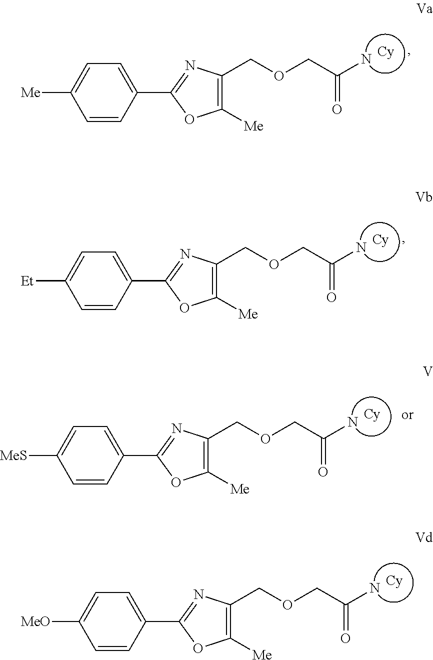 [SMILES: Cc1ccc(-c2nc(COCC(N)=O)c(C)o2)cc1 ; CCc1ccc(-c2nc(COCC(N)=O)c(C)o2)cc1 ; CSc1ccc(-c2nc(COCC(N)=O)c(C)o2)cc1 ; COc1ccc(-c2nc(COCC(N)=O)c(C)o2)cc1]
or a pharmaceutically acceptable salt, solvate or prodrug thereof;
and stereoisomers, isotopic variants and tautomers thereof;
wherein Cy is 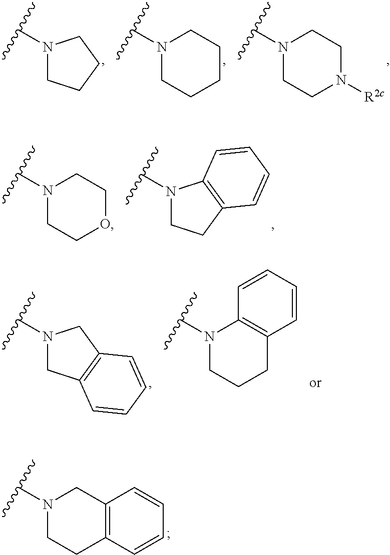
and wherein R^{2c} is H or alkyl.

64. The method according to claim 1 or the compound according to claim 2, wherein the compound is according to formula VIa, VIb, VIc or VId: 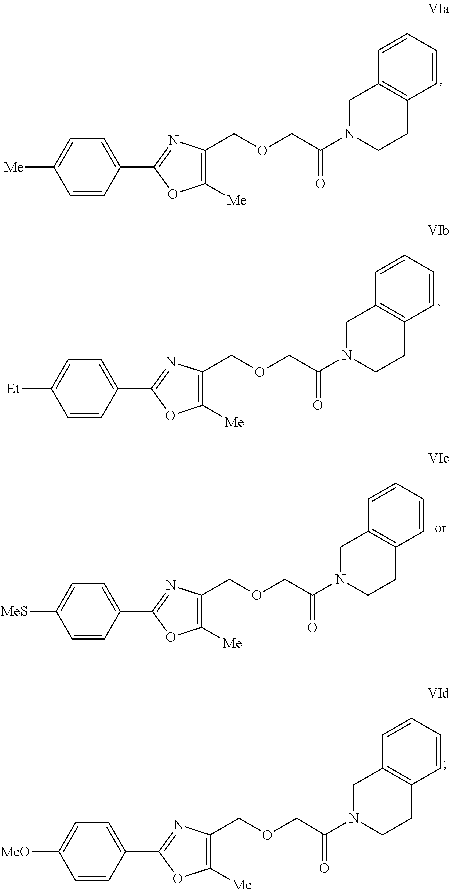 [SMILES: Cc1ccc(-c2nc(COCC(=O)N3CCc4ccccc4C3)c(C)o2)cc1 ; CCc1ccc(-c2nc(COCC(=O)N3CCc4ccccc4C3)c(C)o2)cc1 ; CSc1ccc(-c2nc(COCC(=O)N3CCc4ccccc4C3)c(C)o2)cc1 ; COc1ccc(-c2nc(COCC(=O)N3CCc4ccccc4C3)c(C)o2)cc1]
or a pharmaceutically acceptable salt, solvate or prodrug thereof;
and stereoisomers, isotopic variants and tautomers thereof.

65. The method according to claim 1 or the compound according to claim 2, wherein the compound is according to formula VIIa, VIIb, VIIc or VIId: 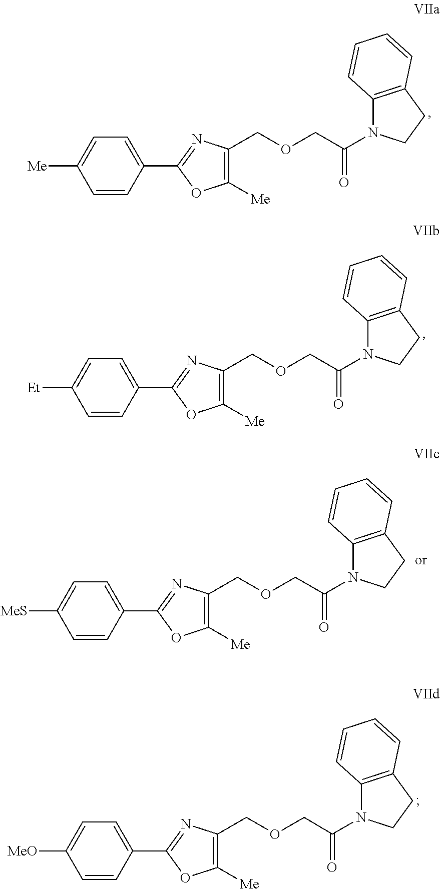 [SMILES: Cc1ccc(-c2nc(COCC(=O)N3CCc4ccccc43)c(C)o2)cc1 ; CCc1ccc(-c2nc(COCC(=O)N3CCc4ccccc43)c(C)o2)cc1 ; CSc1ccc(-c2nc(COCC(=O)N3CCc4ccccc43)c(C)o2)cc1 ; COc1ccc(-c2nc(COCC(=O)N3CCc4ccccc43)c(C)o2)cc1]
or a pharmaceutically acceptable salt, solvate or prodrug thereof;
and stereoisomers, isotopic variants and tautomers thereof.

66. The method according to claim 1 or the compound according to claim 2, wherein the compound is according to formula VIIIa, VIIIb, VIIIc or VIIId: 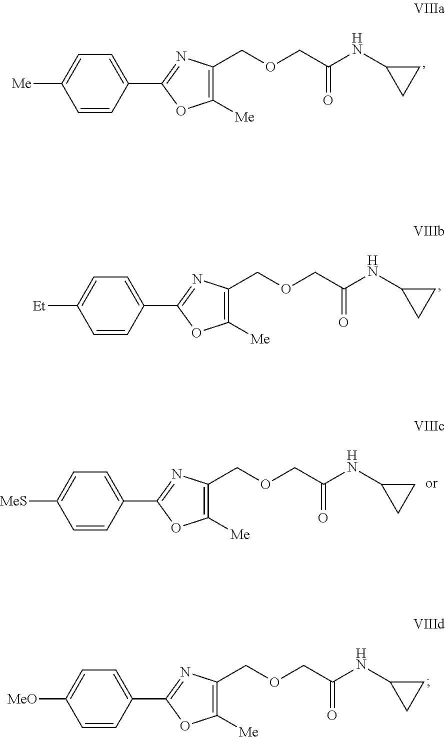 [SMILES: Cc1ccc(-c2nc(COCC(=O)NC3CC3)c(C)o2)cc1 ; CCc1ccc(-c2nc(COCC(=O)NC3CC3)c(C)o2)cc1 ; CSc1ccc(-c2nc(COCC(=O)NC3CC3)c(C)o2)cc1 ; COc1ccc(-c2nc(COCC(=O)NC3CC3)c(C)o2)cc1]
or a pharmaceutically acceptable salt, solvate or prodrug thereof;
and stereoisomers, isotopic variants and tautomers thereof.

67. The method according to claim 1 or the compound according to claim 2, wherein the compound is according to formula IXa, IXb, IXc or IXd: 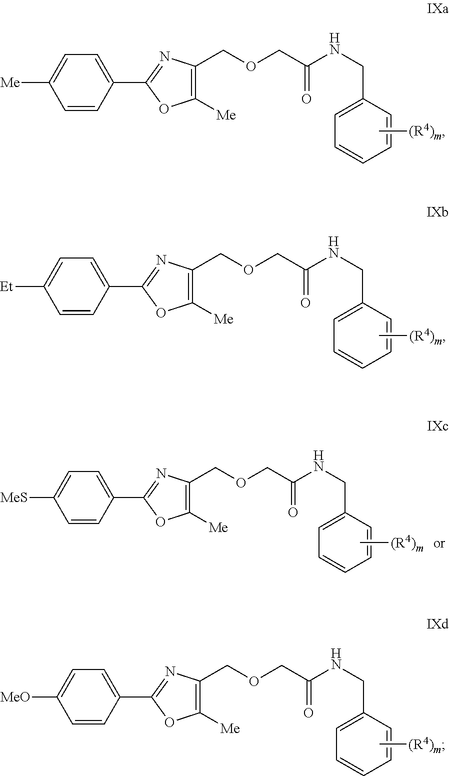
or a pharmaceutically acceptable salt, solvate or prodrug thereof;
and stereoisomers, isotopic variants and tautomers thereof;
wherein m is 1, 2, 3, 4, or 5; and
each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

68. The method according to claim 1 or the compound according to claim 2, wherein the compound is according to formula Xa, Xb, Xc or Xd: 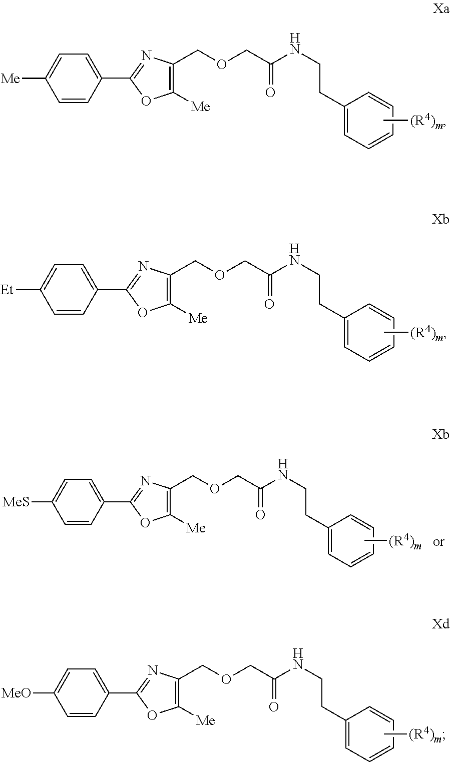
or a pharmaceutically acceptable salt, solvate or prodrug thereof;
and stereoisomers, isotopic variants and tautomers thereof;
wherein m is 1, 2, 3, 4, or 5; and
each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

69. The method or the compound according to either of claim 67 or 68, wherein m is 1 or 2; and each R⁴ is independently Me, Et, i-Pr, OMe, OEt, O-i-Pr, Cl, or F.

70. The method or the compound according to either of claim 67 or 68, wherein each R⁴ is H.

71. The method or the compound according to either of claim 67 or 68, wherein m is 1; and R⁴ is Me, Et, i-Pr, OMe, OEt, O-i-Pr, Cl, or F.

72. The method a or the compound according to either of claim 67 or 68, wherein m is 1; and R⁴ is 4-Cl, or 4-F.

73. The method according to claim 1 or the compound according to claim 2, wherein the compound is according to formula XIa, XIb, XIc or XId: 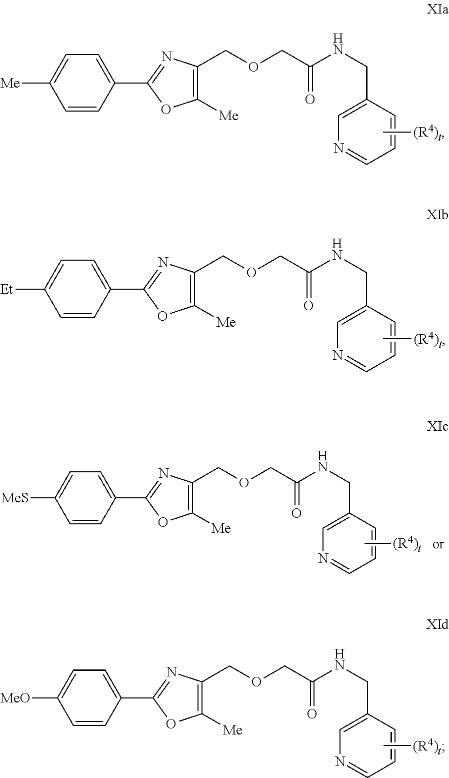
or a pharmaceutically acceptable salt, solvate or prodrug thereof;
and stereoisomers, isotopic variants and tautomers thereof;
wherein t is 1, 2, 3, 4, or 5; and
each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

74. The method according to claim 1 or the compound according to claim 2, wherein the compound is according to formula XIIa, XIIb, XIII or XIId: 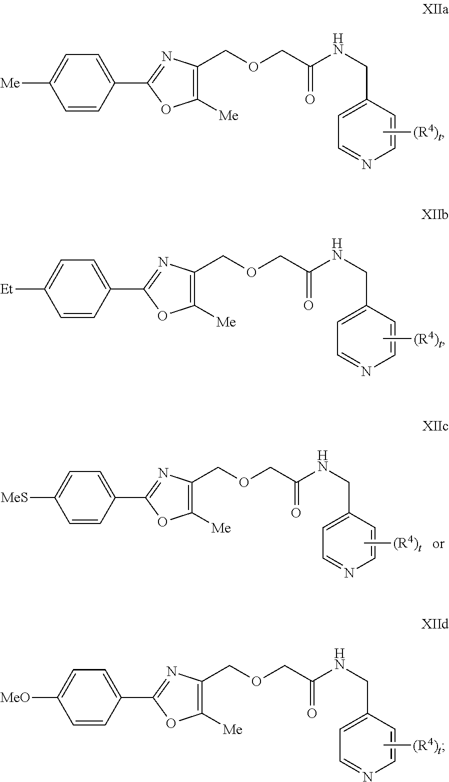
or a pharmaceutically acceptable salt, solvate or prodrug thereof;
and stereoisomers, isotopic variants and tautomers thereof;
wherein t is 1, 2, 3, 4, or 5; and
each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

75. The method according to claim 1 or the compound according to claim 2, wherein the compound is according to formula XIIIa, XIIIb, XIIIc or XIIId: 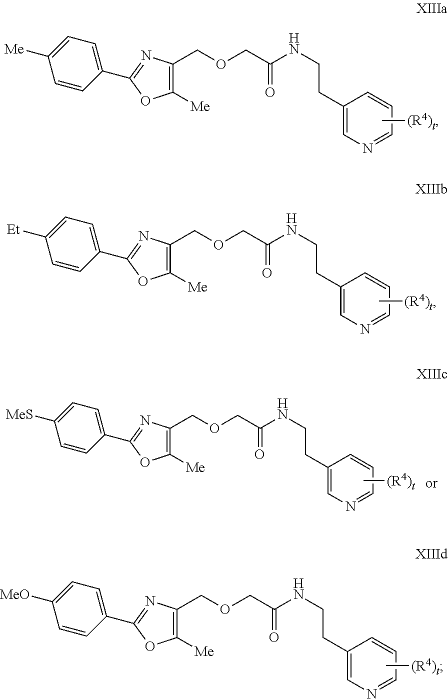
or a pharmaceutically acceptable salt, solvate or prodrug thereof;
and stereoisomers, isotopic variants and tautomers thereof;
wherein t is 1, 2, 3, 4, or 5; and
each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

76. The method according to claim 1 or the compound according to claim 2, wherein the compound is according to formula XIVa, XIVb, XIVc or XIVd: 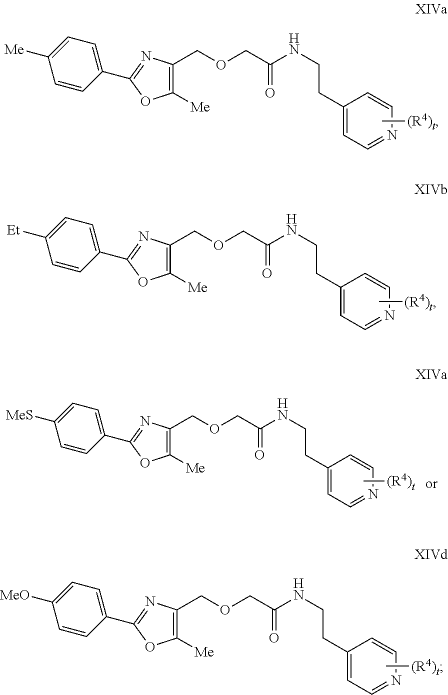
or a pharmaceutically acceptable salt, solvate or prodrug thereof;
and stereoisomers, isotopic variants and tautomers thereof;
wherein t is 1, 2, 3, 4, or 5; and
each R⁴ is independently selected from H, alkyl, substituted alkyl, acyl, substituted acyl, substituted or unsubstituted acylamino, substituted or unsubstituted alkylamino, substituted or unsubstituted alkylthio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfonyl, substituted or unsubstituted sulfinyl, substituted or unsubstituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted heterocycloalkyl, substituted or unsubstituted dialkylamino, halo, heteroaryloxy, substituted or unsubstituted heteroaryl, substituted or unsubstituted heteroalkyl, hydroxy, nitro, and thiol.

77. The method or the compound according to any one of claims 73-76, wherein t is 1 or 2; and each R⁴ is independently Me, Et, i-Pr, OMe, OEt, O-i-Pr, Cl, or F.

78. The method or the compound according to any one of claims 73-76, wherein each R⁴ is H.

79. The method or the compound according to any one of claims 73-76, wherein t is 1; and R⁴ is Me, Et, i-Pr, OMe, OEt, O-i-Pr, Cl, or F.

80. The method according to claim 1 or the compound according to claim 2, wherein the compound is according to formula XVa, XVb, XVc or XVd: 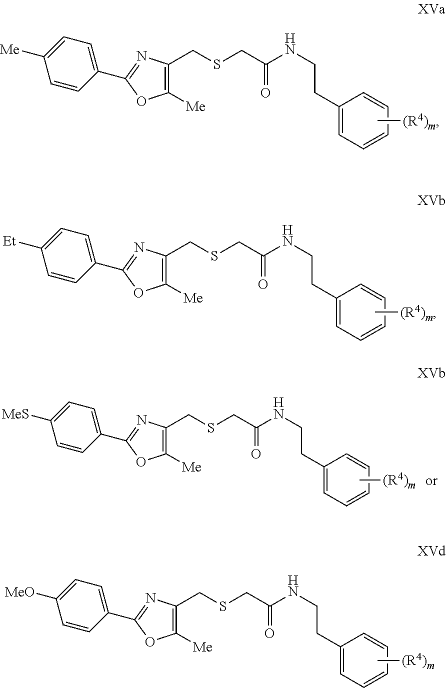
or a pharmaceutically acceptable salt, solvate or prodrug thereof;
and stereoisomers, isotopic variants and tautomers thereof;
wherein m is 1, 2, 3, 4, or 5; and
each R⁴ is independently F or CF₃.

81. The method or the compound according to claim 80, wherein m is 1 and R⁴ is 4-F or 4-CF₃.

82. The method according to claim 1 or the compound according to claim 2, wherein the compound is according to formula XVIa, XVIb, XVIc or XVId: 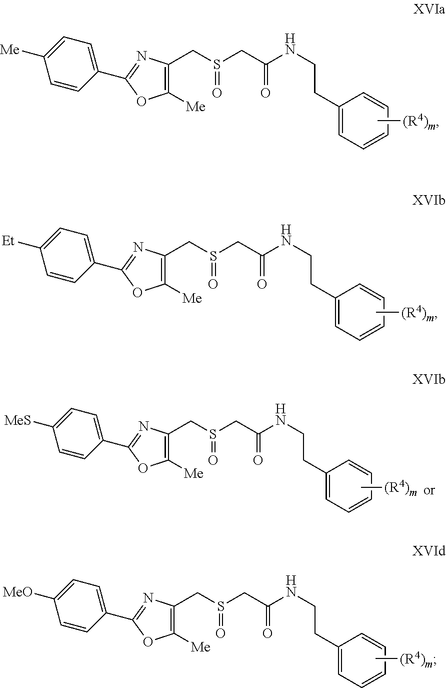
or a pharmaceutically acceptable salt, solvate or prodrug thereof;
and stereoisomers, isotopic variants and tautomers thereof;
wherein m is 1, 2, 3, 4, or 5; and
each R⁴ is independently F or CF₃.

83. The method or the compound according to claim 82, wherein m is 1 and R⁴ is 4-F.

84. The method or the compound according to claim 1 or the compound according to claim 2, wherein the compound is according to formula XVIIa, XVIIb, XVIIc or XVIId: 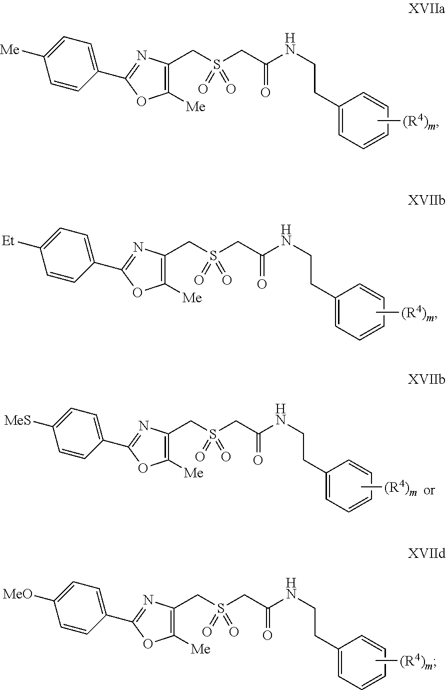
or a pharmaceutically acceptable salt, solvate or prodrug thereof;
and stereoisomers, isotopic variants and tautomers thereof;
wherein m is 1, 2, 3, 4, or 5; and
each R⁴ independently F or CF₃.

85. The method or the compound according to claim 82, wherein m is 1 and R⁴ is 4-F or 4-CF₃.

86. The method according to claim 1 or the compound according to claim 2, wherein the compound is according to formula XVIIIa, XVIIIb, XVIIIc or XVIIId: 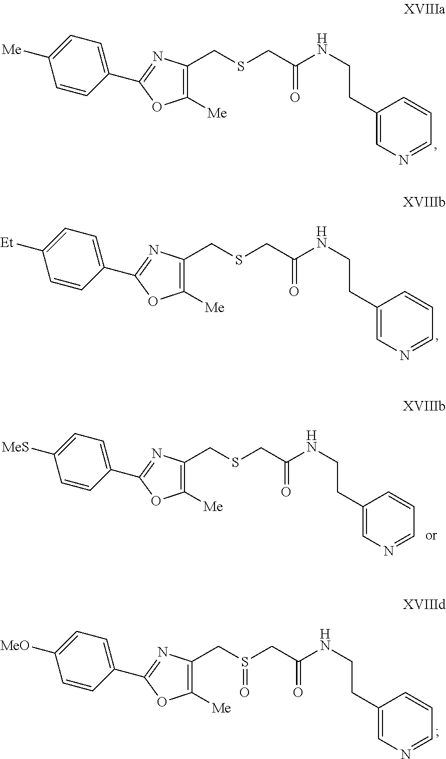 [SMILES: Cc1ccc(-c2nc(CSCC(=O)NCCc3cccnc3)c(C)o2)cc1 ; CCc1ccc(-c2nc(CSCC(=O)NCCc3cccnc3)c(C)o2)cc1 ; CSc1ccc(-c2nc(CSCC(=O)NCCc3cccnc3)c(C)o2)cc1 ; COc1ccc(-c2nc(CS(=O)CC(=O)NCCc3cccnc3)c(C)o2)cc1]
or a pharmaceutically acceptable salt, solvate or prodrug thereof;
and stereoisomers, isotopic variants and tautomers thereof.

87. The method or the compound according to claim 1 or the compound according to claim 2, wherein the compound is according to formula XIXa, XIXb, XIXc or XIXd: 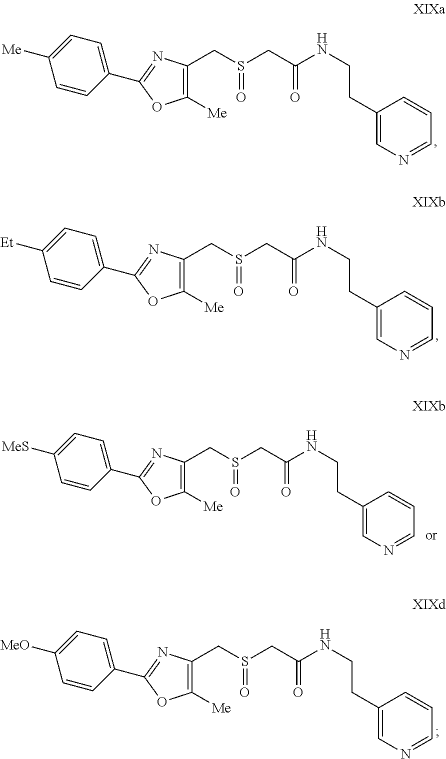 [SMILES: Cc1ccc(-c2nc(CS(=O)CC(=O)NCCc3cccnc3)c(C)o2)cc1 ; CCc1ccc(-c2nc(CS(=O)CC(=O)NCCc3cccnc3)c(C)o2)cc1 ; CSc1ccc(-c2nc(CS(=O)CC(=O)NCCc3cccnc3)c(C)o2)cc1 ; COc1ccc(-c2nc(CS(=O)CC(=O)NCCc3cccnc3)c(C)o2)cc1]
or a pharmaceutically acceptable salt, solvate or prodrug thereof;
and stereoisomers, isotopic variants and tautomers thereof.

88. The method according to claim 1 or the compound according to claim 2, wherein the compound is according to formula XXa, XXb, XXc or XXd: 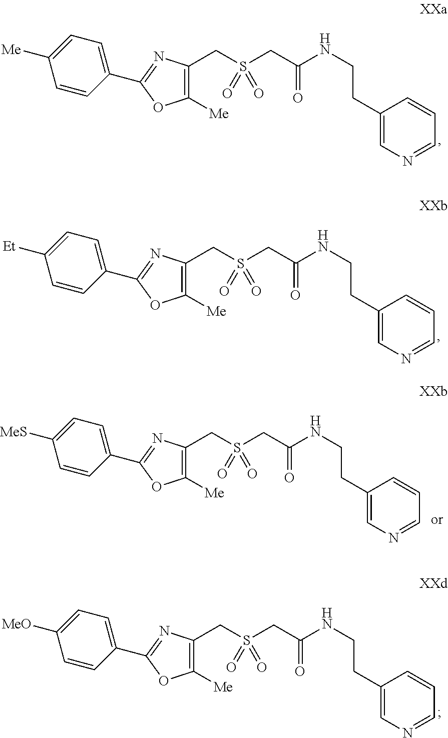 [SMILES: Cc1ccc(-c2nc(CS(=O)(=O)CC(=O)NCCc3cccnc3)c(C)o2)cc1 ; CCc1ccc(-c2nc(CS(=O)(=O)CC(=O)NCCc3cccnc3)c(C)o2)cc1 ; CSc1ccc(-c2nc(CS(=O)(=O)CC(=O)NCCc3cccnc3)c(C)o2)cc1 ; COc1ccc(-c2nc(CS(=O)(=O)CC(=O)NCCc3cccnc3)c(C)o2)cc1]
or a pharmaceutically acceptable salt, solvate or prodrug thereof;
and stereoisomers, isotopic variants and tautomers thereof.

89. The method according to claim 1 or the compound according to claim 2, wherein the compound is according to formula XXIa, XXIb, XXIc or XXId: 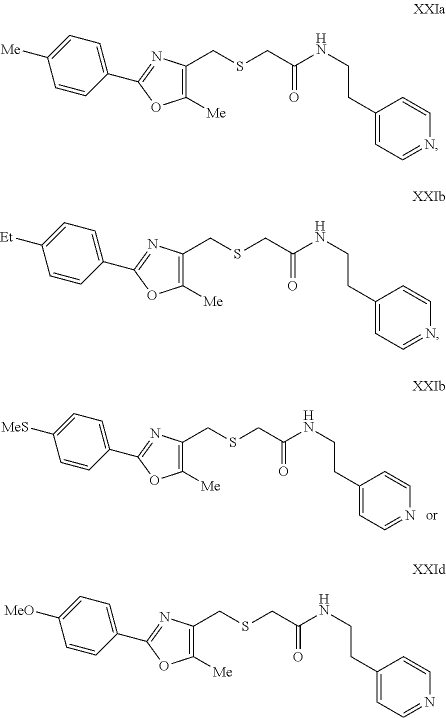 [SMILES: Cc1ccc(-c2nc(CSCC(=O)NCCc3ccncc3)c(C)o2)cc1 ; CCc1ccc(-c2nc(CSCC(=O)NCCc3ccncc3)c(C)o2)cc1 ; CSc1ccc(-c2nc(CSCC(=O)NCCc3ccncc3)c(C)o2)cc1 ; COc1ccc(-c2nc(CSCC(=O)NCCc3ccncc3)c(C)o2)cc1]
or a pharmaceutically acceptable salt, solvate or prodrug thereof;
and stereoisomers, isotopic variants and tautomers thereof.

90. The method according to claim 1 or the compound according to claim 2, wherein the compound is according to formula XXIIa, XXIIb, XXIIc or XXIId: 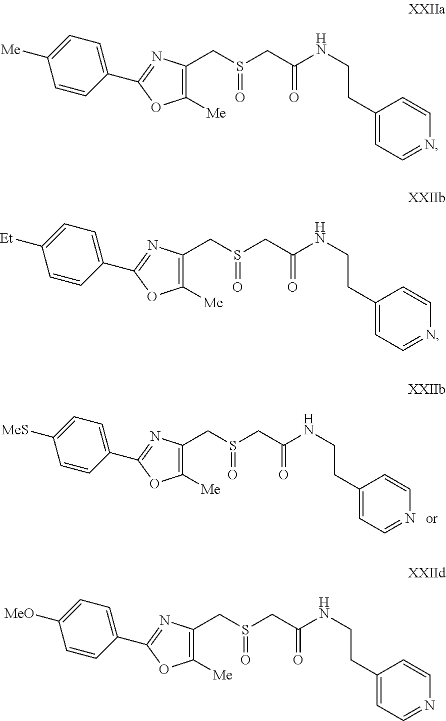 [SMILES: Cc1ccc(-c2nc(CS(=O)CC(=O)NCCc3ccncc3)c(C)o2)cc1 ; CCc1ccc(-c2nc(CS(=O)CC(=O)NCCc3ccncc3)c(C)o2)cc1 ; CSc1ccc(-c2nc(CS(=O)CC(=O)NCCc3ccncc3)c(C)o2)cc1 ; COc1ccc(-c2nc(CS(=O)CC(=O)NCCc3ccncc3)c(C)o2)cc1]
or a pharmaceutically acceptable salt, solvate or prodrug thereof;
and stereoisomers, isotopic variants and tautomers thereof.

91. The method according to claim 1 or the compound according to claim 2, wherein the compound is according to formula XXIIIa, XXIIIb, XXIIIc or XXIIId: 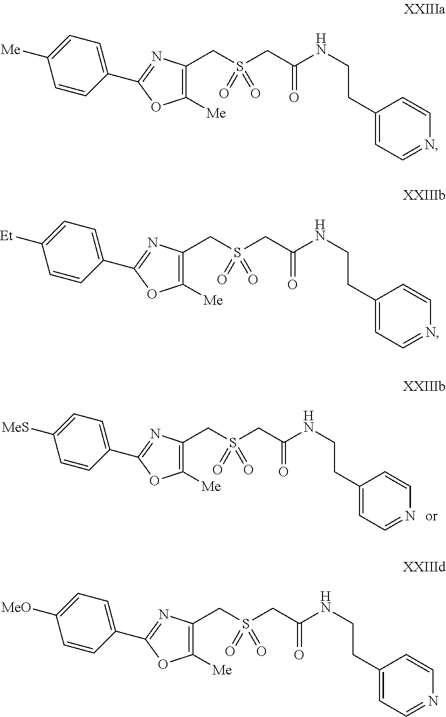 [SMILES: Cc1ccc(-c2nc(CS(=O)(=O)CC(=O)NCCc3ccncc3)c(C)o2)cc1 ; CCc1ccc(-c2nc(CS(=O)(=O)CC(=O)NCCc3ccncc3)c(C)o2)cc1 ; CSc1ccc(-c2nc(CS(=O)(=O)CC(=O)NCCc3ccncc3)c(C)o2)cc1 ; COc1ccc(-c2nc(CS(=O)(=O)CC(=O)NCCc3ccncc3)c(C)o2)cc1]
or a pharmaceutically acceptable salt, solvate or prodrug thereof;
and stereoisomers, isotopic variants and tautomers thereof.

92. The method according to claim 1 or the compound according to claim 2, wherein the compound is according to formula XXIVa′, XXIVb′, XXIVc′ or XXIVd′: 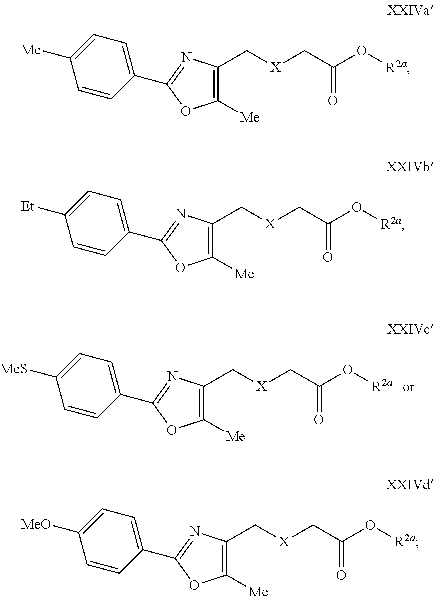
or a pharmaceutically acceptable salt, solvate or prodrug thereof;
and stereoisomers, isotopic variants and tautomers thereof;
wherein R^{2a} is as in claim 1; and X is S, S(O), or S(O)₂.

93. The method according to claim 1 or the compound according to claim 2, wherein the compound is according to formula XXVa′, XXVb′, XXVc′ or XXVd′: 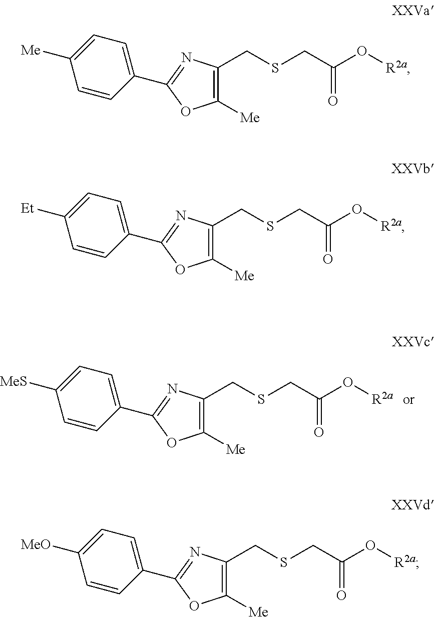
or a pharmaceutically acceptable salt, solvate or prodrug thereof;
and stereoisomers, isotopic variants and tautomers thereof;
wherein R^{2a} is as in claim 1.

94. The method or the compound according to either of claim 92 or 93, wherein R^{2a} is H, substituted or unsubstituted cycloalkyl, substituted or unsubstituted phenyl, substituted or unsubstituted benzyl, or substituted or unsubstituted phenethyl.

95. The method or the compound according to either of claim 92 or 93, wherein R^{2a} is substituted or unsubstituted heteroaryl, or substituted or unsubstituted heterocycloalkyl.

96. The method or the compound according to either of claim 92 or 93, wherein R^{2a} is H, Me, Et, n-Pr, i-Pr, n-Bu, i-Bu, sec-Bu, t-Bu, or cyclopropyl.

97. The method according to claim 1 or the compound according to claim 2, wherein the compound is according to formula XXVIa, XXVIb, XXVIc or XXVId: 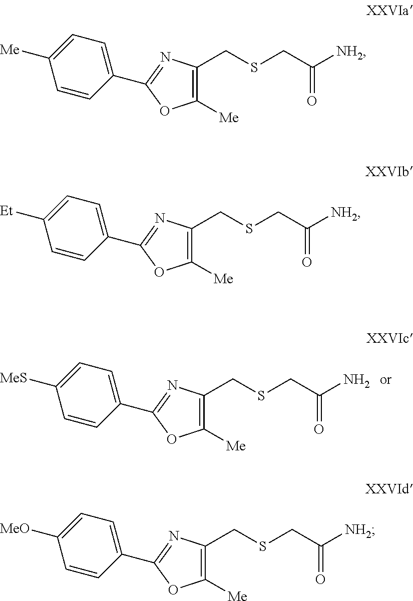 [SMILES: Cc1ccc(-c2nc(CSCC(N)=O)c(C)o2)cc1 ; CCc1ccc(-c2nc(CSCC(N)=O)c(C)o2)cc1 ; CSc1ccc(-c2nc(CSCC(N)=O)c(C)o2)cc1 ; COc1ccc(-c2nc(CSCC(N)=O)c(C)o2)cc1]
or a pharmaceutically acceptable salt, solvate or prodrug thereof;
and stereoisomers, isotopic variants and tautomers thereof.

98. The method according to claim 1 or the compound according to claim 2, wherein the compound is according to formula XXVIa′, XXVIb′, XXVIc′ or XXVId′: 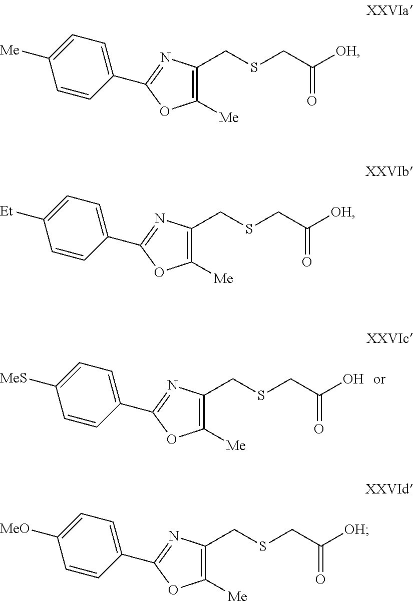 [SMILES: Cc1ccc(-c2nc(CSCC(=O)O)c(C)o2)cc1 ; CCc1ccc(-c2nc(CSCC(=O)O)c(C)o2)cc1 ; CSc1ccc(-c2nc(CSCC(=O)O)c(C)o2)cc1 ; COc1ccc(-c2nc(CSCC(=O)O)c(C)o2)cc1]
or a pharmaceutically acceptable salt, solvate or prodrug thereof;
and stereoisomers, isotopic variants and tautomers thereof.

99. The method according to claim 1 or the compound according to claim 2, wherein the compound is selected from Tables 1-2.

100. The method of any one of claim 1, or 3-98, wherein the disease or condition is cancer.

101. The method of claim 85, wherein the cancer is hepatic cancer, breast cancer, skin cancer, prostate cancer, colon cancer, rectal cancer, head and neck cancer, lung cancer, gastric cancer, mesothelioma, Barrett's esophagus, synovial sarcoma, cervical cancer, endometrial ovarian cancer, Wilm's tumor, bladder cancer or leukemia.

102. A pharmaceutical composition comprising a pharmaceutically acceptable carrier and a pharmaceutically effective amount of a compound of any of claims 2-98.

103. The pharmaceutical composition of claim 102 wherein the carrier is a parenteral carrier, oral or topical carrier.

104. Use of a compound set forth in any of claims 1-98 for the preparation of a medicament for the treatment of a disease or condition that is causally related to the aberrant activity of the Wnt signaling pathway in vivo.

105. Use according to claim 104, wherein the disease or condition is cancer or pulmonary fibrosis.

106. Use according to claim 105, wherein the cancer is hepatic cancer, breast cancer, skin cancer, prostate cancer, colon cancer, rectal cancer, head and neck cancer, lung cancer, gastric cancer, mesothelioma, Barrett's esophagus, synovial sarcoma, cervical cancer, endometrial ovarian cancer, Wilm's tumor, bladder cancer or leukemia.

107. Use according to claim 106, wherein the skin cancer is melanoma, the liver cancer is hepatocellular cancer or hepatoblastoma, and/or the lung cancer is non-small cell lung cancer.

108. Use according to claim 104, wherein the disease or condition is pulmonary fibrosis.

109. A compound as set forth in any one of claims 2-98 for use in the treatment of a disease or condition that is causally related to the aberrant activity of the Wnt signaling pathway in vivo.
